(19) United States
(12) Patent Application Publication
(10) Pub. No.: US 2012/0177611 A1
(43) Pub. Date: Jul. 12, 2012

(54) Elastic substrates and methods of use in cell manipulation and culture

(76) Inventors: Helen M. Blau, Menlo Park, CA (US); Penney M. Gilbert, Menlo Park, CA (US); Karen Havenstrite, Menlo Park, CA (US); Matthias P. Lutolf, Tolochenaz (CH); Klas Erik Gustaf Magnusson, Nacka (SE); John A. Ramunas, Stanford, CA (US)

(21) Appl. No.: 13/135,826

(22) Filed: Jul. 15, 2011

Related U.S. Application Data
(60) Provisional application No. 61/399,698, filed on Jul. 15, 2010.

(51) Int. Cl. A61K 35/12; G06K 9/62; C12Q 1/02; C12M 3/00; C12N 5/071; C12N 5/0735

(52) U.S. Cl. 424/93.7; 435/325; 435/374; 435/29; 435/289.1; 382/133

(57) ABSTRACT

Methods are provided for the ex vivo manipulation of cells, stem cells and other reproductive cells, by manipulating the cells in a container or device comprising an elastic substrate, wherein the substrate has an elasticity that mimics the elasticity of a native microenvironment of the cell.

### GOVERNMENT RIGHTS

This invention was made with government support under CA09151, 2 T32 HD007249, HL096113, and AG020961 awarded by the NIH; and TG2-01159, RB1-01292, and RT1-01001 awarded by the California Institute of Regenerative Medicine. The Government has certain rights in the invention.

### BACKGROUND OF THE INVENTION

Stem cells have a capacity both for self-renewal and the generation of differentiated cell types, which provides the possibility for therapeutic regeneration of cells and tissues in the body. In addition to studying the important normal function of stem cells in the regeneration of tissues, researchers have further sought to exploit the potential of in situ and/or exogenous stem cells for the treatment of a variety of disorders. While early, embryonic stem cells have generated considerable interest, the stem cells resident in adult tissues also provide an important source of regenerative capacity.

These somatic, or adult, stem cells are undifferentiated cells that reside in differentiated tissues, and have the properties of both self-renewal and generation of differentiated cell types. The differentiated cell types may include all or some of the specialized cells in the tissue. For example, hematopoietic stem cells give rise to all hematopoietic lineages, but do not seem to give rise to stromal and other cells found in the bone marrow. Sources of somatic stem cells include, but are not limited to, bone marrow, blood, the cornea and the retina of the eye, brain, skeletal muscle, cartilage, bones, dental pulp, liver, kidney, heart, skin, the lining of the gastrointestinal tract, and pancreas, and the like. Adult stem cells are usually quite sparse. Often they are difficult to identify, isolate, and purify. Often, somatic stem cells are quiescent until stimulated by the appropriate growth signals.

Somatic stem cells in vivo reside in so-called “niches” or protective microenvironments that are composed of complex mixtures of signaling proteins. A specific microenvironment, or niche, has been shown to play a critical role in the maintenance of stem cell functions, for example see Spradling et al., Nature 414: 98 (2001); Fuchs et al., Cell 116: 769 (2004); Moore & Lemischka, Science 311: 1880 (2006); Scadden, Nature 441: 1075 (2006). Many essential signals may be membrane-bound and thus conformationally controlled and immobilized on supportive cells in close physical contact with adult stem cells. These signals direct stem cell behavior by different means, including, but not limited to, protecting them from differentiation, influencing the cell cycle (e.g., maintaining quiescence) and self-renewal divisions. In the absence of cross-talk with their respective natural niche, as is the case with in vitro culture, adult stem cells rapidly differentiate and lose their multipotentiality.

For example, muscle tissue in adult vertebrates regenerates from stem cells known as satellite cells. Satellite cells are distributed throughout muscle tissue and are mitotically quiescent in the absence of injury or disease, residing in an instructive, anatomically defined niche. The satellite cell niche constitutes a distinct membrane-enclosed compartment within the muscle fiber, containing a diversity of biochemical and biophysical signals that influence satellite cell function. A major limitation to the study and clinical utility of satellite cells is that upon removal from the muscle fiber and plating in traditional plastic tissue culture platforms, their muscle stem cell properties are rapidly lost. Clearly, the maintenance of stem cell function is critically dependent on in vivo niche signals, highlighting the need to create novel in vitro microenvironments that allow for the maintenance and propagation of satellite cells while retaining their potential to function as muscle stem cells. In addition to satellite cells, cell types that contribute to muscle regeneration include, but are not limited to, mesangioblasts, bone marrow derived cells, muscle interstitial cells, mesenchymal stem cells. See D. D. Cornelison et al. (2001) Dev Biol 239, 79; S. Fukada et al. (2004) Exp Cell Res 296, 245; D. Montarras et al. (2005) Science 309, 2064; S. Kuang et al. (2007) Cell 129, 999; M. Cerletti et al. (2008) Cell 134, 37; C. A. Collins et al. (2005) Cell 122, 289; A. Sacco et al. (2008) Nature 456, 502; R. I. Sherwood et al. (2004) Cell 119, 543; and Galvez et al. (2006) J Cell Biol. 174(2):231-43

Many types of tissue-resident stem cells have remarkable regenerative capacity when freshly isolated, a property that is rapidly lost once plated in culture. Indeed, the propagation of functional adult stem cells is currently not possible in culture, despite extensive research on biochemical signals. Methods of culturing various stem cells in a way that promotes their regenerative capacity are therefore in great need, as are methods of culturing other types of cells. In addition to somatic stem cells, the culture of oocytes and early stage embryos is of particular interest. Assisted reproductive technologies (ART) comprise a set of tools and techniques, including in vitro fertilization (IVF), that can overcome infertility due to various causes, including low sperm number or quality, compromised fallopian tube structure or function, endometriosis, diminished ovarian reserve, and others. ART also enables surrogacy, in which an oocyte is fertilized and then carried to term in the uterus of a woman other than the oocyte donor. ART often begins with ovarian stimulation, in which hormones are taken to cause multiple follicles to mature so that multiple oocytes are released during ovulation. The oocytes are collected through a needle which pierces the vaginal wall to access an ovary. The oocytes may then be cultured with sperm in vitro to cause fertilization, or a sperm can be injected directly into an oocyte in intracytoplasmic sperm injection (ICSI). The resulting conceptus (a sperm-penetrated oocyte or any of its derivatives up to birth), is then typically cultured in vitro for a few days to allow it to develop to the stage at which embryos normally arrive at the uterus. The embryo or blastocyst is then transferred to the uterus of a recipient.

Current cell culture ware used in IVF, ICSI, and other ART techniques are usually made of plastic or glass, materials with very different elasticities than tissues, forcing cells to cope with unnatural physical and chemical conditions, reducing the probability of successful pregnancy, and causing deleterious changes, for example in epigenetic imprinting. For example, the success rate of ART, including IVF and ICSI, is less than 50% for women under 35 and rapidly drops to less than 20% over age 40. Methods that improve the success rate are of great interest.

The present invention addresses the expansion of cells (including tissue-derived stem cells, progenitor cells, reproductive cells, somatic cells, etc.) on an elastic substrate. In addition to tissue-specific stem cells, a culture condition that allows for stem cell propagation could be utilized for embryonic stem cells and induced pluripotent cells (iPS), which often require coculture with feeder cells. The viability of fertilized oocytes (in vitro fertilization) and other rare cell types may be increased by such culture methods. Cells of interest include all eukaryotic cells, usually plant or animal cells. Further, the directed differentiation of stem cells toward a tissue specific fate, e.g. mature neurons or cardiomyocytes may be enhanced by such culture conditions.

### SUMMARY OF THE INVENTION

Compositions and methods are provided for cell manipulations, including culture in vitro, on soft hydrogels of defined composition, where the hydrogel substrate provides for increased viability and expansion of cells. Cell culture methods of interest comprise, without limitation, the transfer, maintenance and culture of reproductive cells, including, but not limited to, germ cells, gametocytes, gametes, oocytes, fertilized oocytes, zygotes and early stage embryos; maintenance and expansion of somatic stem cells; maintenance and expansion of multipotent or pluripotent cells, including, but not limited to, induced pluripotent stem cells (iPS), embryonic stem cells; reprogrammed cells, transdifferentiated cells, embryonic stem cells (ES cells) and dedifferentiated cells; maintenance and expansion of primary cells and cell lines; maintenance and expansion of differentiated cells, somatic cells; and the like. Cell manipulations of interest also include the use of substrates of the invention as a medium for cell transplantation in vivo, where calls may be attached, encapsulated, and the like to a substrate.

In some embodiments the invention comprises methods and systems for increasing the probability of successful pregnancies resulting from IVF, ICSI, and other ART techniques, as well as transgenic animal creation and animal cloning, by providing cell culture methods, tools, and systems that mimic elements of native environments which oocytes, sperm, cumulus-oocyte complexes, or concepti encounter in vivo. Culture containers and tools are provided that comprise elastic substrates with elasticities that approximate the elasticities of these native environments.

In some aspects of the invention, methods are provided for the expansion of cells, including somatic stem cells in culture. In the methods of the invention, the cells are seeded in vitro on the surface of a hydrogel substrate, where the substrate has an elasticity of a physiological substrate, for example an elasticity that is matched to the elasticity of the tissue from which the cells are derived. In some methods of the invention, the elasticity of the hydrogel substrate is varied before, during, or after the seeding of the cells. In some cases, the elasticity is varied over time while the cells are in culture. The manipulated cells may be used for regenerative purposes.

In some embodiments the hydrogel further comprises at least one polypeptide, biomolecule or chemical component, e.g. a structural or soluble protein. The expanded stem cell population may be utilized for in vivo purposes, including, but not limited to, transplantation for cell based therapies; for various screening purposes in vitro, and the like.

The cells may be maintained in culture for a period of time sufficient to increase the number of cells having a stem cell phenotype, for example increasing the number of stem cells by at least 1.5 fold, at least two-fold, at least three-fold, at least five-fold, or more. Cultures may be maintained for a period of time sufficient to provide for the parameters of interest, for example at least about an hour, about 2 hours, about 6 hours, about 1 day, about 1 week, about 2 weeks, about 3 weeks, about 4 weeks, about 5 weeks, or more. Stem cell phenotypes of interest include, without limitation, the ability to participate in the development of the cognate tissue in vivo; display of cell surface markers shown to be associated with the cognate stem cell; ability to give rise in vitro to colonies of the differentiated cell types associated with the stem cell; rate of self-renewal and the like.

Various factors can be utilized to tune the elasticity of the substrate, including selection of hydrogel components, concentration of polymers with respect to the aqueous component, length, branching and cross-linking of the polymers, and the like. Determination of the elasticity of the substrate may be performed using conventional methods to determine the optimum conditions for a given cell.

In certain embodiments of the invention, the stem cell is a somatic cell. In some cases, the somatic cell is a muscle stem cell, which cells are shown herein to often require for proliferation a highly elastic substrate that mimics the mechanical properties of native muscle tissue. The substrate for muscle stem cell expansion may further comprise at least one protein component of the muscle tissue, or niche, e.g. laminin, fibronectin, etc. It is shown herein that muscle stem cells cultured by the methods of the invention are able to contribute to in vivo tissue regeneration. The expanded population of muscle stem cells is useful in transplantation, particularly for the regeneration of skeletal muscle, e.g. in the treatment of muscle disorders such as heritable or acquired muscular dystrophies, myopathies, chanelopathies, sarcopenia; following traumatic damage; and the like. The cells are also useful for experimental evaluation, and as a source of lineage and cell specific products, including mRNA species useful in identifying genes specifically expressed in these cells, and as targets for the discovery of factors or molecules that can affect them. The discovery of RNAs and proteins may yield new drug targets. The discovery of factors or molecules that affect stem cell self-renewal or differentiation or cell function may yield new drugs.

In some embodiments, the cells (e.g., stem cells, dedifferentiated cells, etc.) may be used for reconstitution or regeneration of tissue. In some cases, cultured muscle stem cells may be used for reconstitution of muscle function in a recipient. Allogeneic or autologous cells may be used for progenitor cell isolation and subsequent transplantation, for example where the disease conditions result from genetic defects in the function of a particular organ or organ system, (e.g., muscle cell function). Where the dysfunction (e.g., muscle dysfunction) arises from conditions such as trauma, atrophy due to immobilization, muscle wasting due to other illness (eg. cancer) or aging, the subject cells may be isolated from autologous tissue, and used to regenerate function. Autologous cells may also be genetically modified, in order to correct disease conditions results from genetic defects.

In other embodiments, software and methods are provided for tracking individual cells in culture. The trajectories of individual cells are tracked, and the unique identity of individual cells is determined through the tracking, so that parameters of individual cells can be extracted from the data. The software provides the data processing necessary to analyze cell behavior, from preprocessing images to automatically tracking cells and determining cell trajectories, division kinetics, viability, and geneology.

### BRIEF DESCRIPTION OF THE DRAWINGS

The invention is best understood from the following detailed description when read in conjunction with the accompanying drawings. The patent or application file contains at least one drawing executed in color. Copies of this patent or patent application publication with color drawing(s) will be provided by the Office upon request and payment of the necessary fee. It is emphasized that, according to common practice, the various features of the drawings are not to-scale. On the contrary, the dimensions of the various features are arbitrarily expanded or reduced for clarity. Included in the drawings are the following figures.

FIG. 1. Pliant hydrogel promotes MuSC survival and prevents differentiation in culture. (A) PEG hydrogels with tunable mechanical properties. Young's modulus is linearly correlated with precursor polymer concentration; (E; n=4) red circle indicates muscle elasticity. (B) Image of a pliant PEG hydrogel on a green spatula. Scale bar, 7 mm (top). Confocal immunofluorescence image of hydrogel microcontact printed with laminin specifically at the bottom of hydrogel microwells (i.e. from the ‘tips’ of the micropillars). Scale bar, 125 μm (bottom). (C) Dissected tibialis anterior muscles (n=5 animals, 10 muscles total) were analyzed by rheometry (horizontal line indicates the mean). (D) Gel surface protein density did not differ significantly on PEG hydrogels of different rigidities (Young's modulus, E; p>0.05) and was 7.6 ng/cm2±1.0 ng/cm2 (n≧4). (E) Scheme of Baxter Algorithm analysis of timelapse videos. Hydrogel arrays with hundreds of microwells containing single MuSCs were followed by timelapse microscopy for 3 days. Videos were automatically processed and analyzed (Supplemental Methods). Scale bar, 100 μm. (F) Single MuSC (black data points) velocity on pliant or rigid culture substrates. Circles denote mean velocity±standard deviation (p<0.0001). (G) Change in total MuSC number on soft (top plot) or stiff (bottom plot) substrates during timelapse acquisition. Deaths (X) and divisions (O) are shown and colors designate five cell generations (G1-G5). The proportion of cells in each generation at all timepoints is shown. Cell number is normalized to a starting population of 100 single MuSCs.

FIG. 2. Cultured MuSC engraftment is modulated by substrate elasticity. (A) Scheme of in vivo transplantation experiments. (B) Scatter graph of BLI values of recipient mice one month after transplantation with 100 GFP/Fluc MuSCs after 7 day culture on substrates of varying stiffness (left; n=15). Representative bioluminescence images of animals transplanted with each culture condition are shown (right; photons/sec/cm2/sr). (C) Percentage of animals from each experimental condition that had a BLI value above the engraftment threshold. Fisher's exact test p<0.05. (D) Scatter graph of BLI values of recipient mice one month after transplantation with different numbers of Fluc MuSCs cultured for 7 days on either hydrogel (black) or plastic (red). Representative bioluminescence images of animals transplanted with each culture condition are shown (right; photons/sec/cm²/sr). (E) Percentage of total transplanted animals in each experimental condition exhibiting a BLI value above the engraftment threshold.

FIG. 3. Culture on pliant hydrogel promotes muscle stem cell engraftment and niche repopulation in vivo. (A) Engraftment of freshly isolated (black line, squares; 500 cells) and pliant (green line, circles; 1500 cells) or rigid (red line, diamonds; 1500 cells) substrate cultured MuSCs monitored by BLI for a period of 30 days post transplantation (p<0.05). (B) Immunofluorescence of GFP expression in transverse sections of muscles one month after transplantation with freshly isolated (left; 500 transplanted cells) or one week pliant hydrogel cultured MuSCs (right; 2500 transplanted cells). GFP=green, Laminin=red, and Hoechst=blue. Scale bar, 100 μm. (C) Immunohistochemical analysis of transverse muscle tissues one month after transplant with pliant hydrogel cultured MuSCs (right; 2500 transplanted cells). GFP=green, Laminin=red, and Hoechst=blue. Scale bar, 100 μm. (C) Immunohistochemical analysis of transverse muscle tissues one month after transplant with pliant substrate cultured MuSCs. Arrow points to a donor derived cell in satellite cell position. β-galactosidase=green, Laminin=red, and Hoechst=blue. Scale bar, 50 μm.

FIG. 4. Culture on pliant hydrogel promotes muscle stem cell self-renewal. (A) Scheme of in vivo self-renewal assay. (B) Percentage of total doublets exhibiting a Pax7+/+ gene signature in pliant (n=47) or stiff (n=32) microwells (right). Representative image of a Pax7+/+ doublet (left). Native Zs-Green=green (i.e. Pax7), Hoechst=blue, scale bar, 20 μm. (C) Scatter graph (left) of bioluminescent values from mice transplanted with doublets derived from pliant (n=12) or stiff (n=14) microwells or clones collected from pliant microwells (n=8). Images of animals with bioluminescent values above threshold are shown (right; photons/sec/cm²/sr). (D) Immunohistochemistry of GFP expression in transverse sections of muscles one month after transplantation with five MuSC doublets (top; GFP+ fibers persist ˜13 mm longitudinally) or a single clone (bottom; fibers persist ˜7 mm longitudinally) cultured on pliant hydrogel. GFP=green, Laminin=red, and Hoechst=blue. Scale bar, 100 μm.

FIG. 5. Analysis of hydrogel swelling properties. Hydrogels with different mechanical properties were fabricated and weighed before and after overnight equilibration in PBS. Bar graph with hydrogel swelling data displayed as the fold change in mass relative to the hydrogel mass prior to equilibration is shown (n=4).

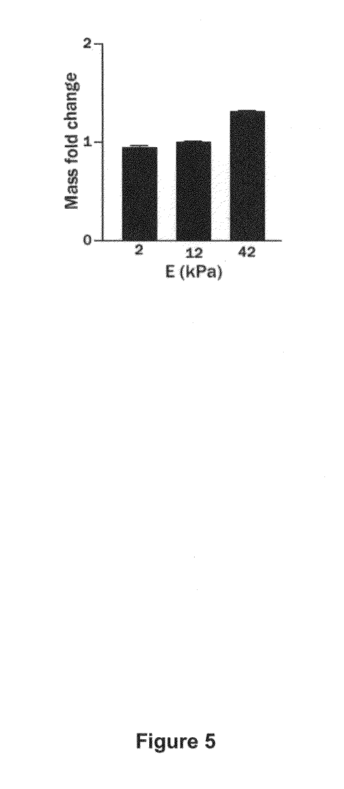

FIG. 6. Automated tracking of MuSC size. A representative example of cell size tracked over the course of a timelapse experiment using the Baxter Algorithm. Data shows a single microwell initially containing a single MuSC (red) that divides (black arrows with cell generation; G) to produce a total of eight cells by the end of the timelapse acquisition (red arrow). Each color corresponds to an individual MuSC. Cell spreading is indicated by sharp spikes in the graph.

FIG. 7. Matrix rigidity regulates MuSC clonal survival in culture. Clonal MuSC viability assessed as the total number of viable clones arising from 1,500 freshly isolated MuSCs cultured for seven days in pliant (white bars) or rigid (black bars) microwells (n=3 independent experiments, p<0.01).

FIG. 8. Automated MuSC survival analysis. Bar graphs indicate the number of live (white bars) and dead (black bars) MuSCs present in arrays of pliant and rigid microwells at the end of timelapse acquisition. The data is normalized to a starting population of 100 single MuSCs and live/dead data for each condition is subdivided to indicate the generation status of cells.

FIG. 9. Automated analysis of clonal cell count and viability. Bar graphs show the final cell count (y-axis) for all analyzed clones (x-axis) observed within pliant (top graph) and rigid (bottom graph) microwell arrays at the end of timelapse acquisition. Each vertical bar represents a single clone and indicates the number of live (black) and dead (white) cells included in the count.

FIG. 10. MuSC differentiation is regulated by substrate stiffness. MuSCs cultured for one week were assayed for Myogenin immunoreactivity, revealing reduced Myogenin expression on pliant (white bars) as opposed to rigid (black bars) substrates (p<0.001).

FIG. 11. MuSC time to first division is not regulated by substrate rigidity. Analysis of timelapse videos using the Baxter Algorithm shows single MuSC time to first division on pliant compared with rigid substrates is not statistically significant in the distribution of the data (12 kPa, 52 hrs±10; ˜10⁶ kPa, 49 hrs±8). Statistical significance was determined using a Wilcoxon rank sum test p>0.05).

FIG. 12. Automated analysis of MuSC time between divisions. Scatter plots showing the time between each division for MuSCs cultured in pliant (top) or rigid (bottom) microwells. Cell generation information is shown on the x-axis. Data indicate no difference in the time between division of MuSCs cultured on soft compared to rigid substrates (12 kPa, 8.5 hrs±1.1; ˜10⁶ kPa, 8.5 hrs±1.7). Statistical significance was determined using a Wilcoxon rank sum test (p>0.05).

FIG. 13. MuSC division rate is not regulated by stiffness. Timelapse data (jagged lines; p<0.01) was used to calculate MuSC division and death rates at a population level. An exponential curve taking into consideration both the death and division rates was generated (solid lines; p<0.0001). An exponential curve that excludes death and takes into account only division shows that the division rate is not significantly (n.s.) different between the two culture conditions (dashed lines; p>0.05).

FIG. 14. Bioluminescence threshold determination. Representative immunofluorescent images of GFP (green) positive donor derived fibers observed in transverse muscle sections from animals with bioluminescence values ranging from 4×10⁴ to 10×10⁴ photons/second, four weeks after transplantation. A bioluminescence readout of ≧80,000 photons/second correlates with observation of donor derived fibers by histology and is used as the threshold value that indicates engraftment in this manuscript. Laminin=red, Hoechst=blue. Scale bar, 100 μm.

FIG. 15. Pax7 immunofluorescence threshold determination. To establish an unbiased immunofluorescence value corresponding to positive Pax7 expression, single MuSCs isolated from Pax7-ZsGreen transgenic animals were spatially segregated in soft (left) or rigid (right) microwell arrays and cultured for 2-3 days to permit a single division event to occur (doublet formation). Native ZsGreen (i.e. Pax7 expression) and Topro (nuclear stain) images for each MuSC were acquired by confocal microscopy. ZsGreen signal for each MuSC was normalized to cell area and nuclear staining intensity. Normalized Pax7 values for each analyzed cell are shown. Hatched line indicates the threshold established by the data, which was used to retrospectively assess Pax7 expression within MuSC doublets.

FIGS. 16A-16C. Culture system containers and devices with elastic substrates.

FIG. 17. Phase images assessing viability and embryonic development stage following culture on hydrogel. Comparison of 42 kPa and 10⁶ kPa elasticities.

FIG. 18. Phase images assessing viability and embryonic development stage following culture on hydrogel. Comparison of 20 kPa, 42 kPa and 10⁶ kPa elasticities.

FIGS. 19A-19B. Analysis of hydrogel. A. MALDI-MS spectrum for 10 kDa PEG-Vinyl sulfone product. The product is highly polydisperse; showing PEG-VS precursors of varying size (3 kDa to 10 kDa molar mass). B. NMR analysis of PEG-VS product.

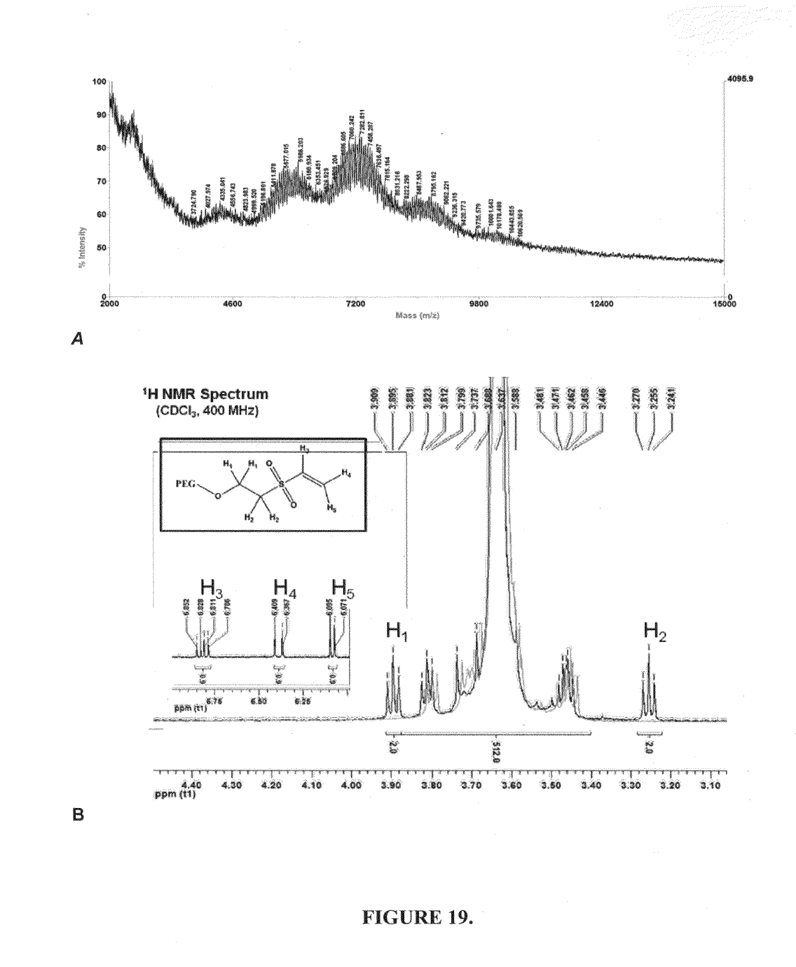

### DESCRIPTION OF THE SPECIFIC EMBODIMENTS

Compositions and methods are provided for the ex vivo manipulation of cells, including, but not limited to, stem cells, reproductive cells, somatic cells, multipotent cells, pluripotent cells, germ cells, primary cells and cell lines. Methods of the invention include, but are not limited to, transfer, maintenance, and expansion of cells in culture by seeding in vitro on a hydrogel substrate, where the substrate has an elasticity of a physiological substrate, for example an elasticity that is matched to the elasticity of the tissue from which the cells are derived. Optionally the hydrogel may further comprise at least one polypeptide, biomolecule or chemical component, e.g. a structural or soluble protein. The manipulated cell population (e.g., stem cell population) may be utilized for in vivo purposes, including, but not limited to, transplantation; tissue regeneration; for various screening purposes in vitro, and the like.

The cultured cells may be experimentally modified prior to, or during the culture period. In some embodiments, the cultured cell is a stem cell. In some embodiments, the stem cells are modified by exposure to viral or bacterial pathogens. In other embodiments cells are modified by altering patterns of gene expression, e.g. by providing reprogramming factors to induce pluripotency or otherwise alter differentiation potential; by introducing factors that provide for oncogenic transformation, by introducing therapeutic genes such as dystrophin, and the like. The experimentally modified cells are useful for investigation of the effects of therapeutic agents for anti-viral or anti-bacterial activity; for tumor therapy, for effects on differentiation, for increasing stem cell numbers, and the like. For example, the effect of a gain or loss of gene activity on the ability of cells to expand in culture may be determined, or on the ability to undergo tumor transformation or to differentiate partially or fully along a specific pathway.

In another aspect of the invention, a method is provided for in vitro screening for agents for their effect on cells expanded using the culture conditions of the present invention. For example, stem cells cultured by the methods described herein are exposed to candidate agents. Agents of interest include, but are not limited to, pharmaceutical and genetic agents, e.g. antisense, expressible coding sequences, RNAi, and the like, where the genetic agents may correspond to candidate tumor suppressors, candidate oncogenes, and the like. In some embodiments, the effect on the cell phenotype is determined. In other embodiments the effect of transformation or growth of tumor cells is determined, for example where agents may include, without limitation, chemotherapy, monoclonal antibodies or other protein-based agents, radiation/radiation sensitizers, cDNA, siRNA, shRNA, small molecules, chemicals and the like. Agents active on stem cells are detected by changes in growth of the cultured cells, e.g. in their ability to participate in in vivo regeneration; expression of markers indicative of stem cells; and the like.

Methods are provided for screening cells in a population, e.g. a complex population of multiple cell types, a population of purified cells isolated from a complex population by sorting, culturing, and the like, for the presence of cells having stem cell potential. In some cases, the method entails culture of candidate cells with the substrate culture of the invention. Candidate cells with stem cell potential are detected by expansion of cells having a stem cell phenotype, as determined by any one of the methods described herein.

In some embodiments, containers and tools with elastic substrates are provided for culturing, maintaining, expanding, proliferating, manipulating, treating, storing, and freezing cells, including, but not limited to, reproductive cells (e.g. gametes, concepti) reproductive tissues (e.g. follicles), stem cells, pluripotent cells, multipotent cells, reprogrammed cells, transdifferentiated cells, dedifferentiated cells, and primary cells. The containers may have any shape, e.g. Petri dish, centrifuge tubes, microcentrifuge tubes, cryovials, multi-well plates, T-flasks, beakers, test tubes, wherein the elasticity of the elastic substrate is physiologically appropriate. The elastic substrate may be flat or similarly planar, or may comprise cilia, striations, dimples, hemispheres, tubes, and the like. A container may comprise a plurality of elastic substrates of different elasticities, for example when co-culturing different cell or tissue types. Tools may include, but are not limited to, pipets, e.g. holding pipets, injection pipets, and the like, where the pipet may have an open or closed lumen.

In some cases, the compositions, devices, or methods of the invention may be used to treat reproductive conditions or may be used in certain reproductive methods, e.g., assisted reproductive technology (ART), in vitro fertilization. ART methods may include, but are not limited to, egg or sperm collection; where the germ cells may be received in a container comprising an elastic substrate; germ cell activation, where sperm or oocytes may be maintained on an elastic substrate of the invention during the activation process; germ cell storage, for example, cryogenesis, in which the cells are maintained in vials comprising an elastic substrate of the invention before during or after freezing; in vitro fertilization in which the germ cells and optional supporting cells may be maintained in container(s) comprising an elastic substrate of the invention; embryo stripping by pipetting using pipets and containers comprising an elastic substrate of the invention; intracytoplasmic sperm injection (ICSI) using holding pipets or containers comprising an elastic substrate of the invention; culture of a conceptus to a stage appropriate for implantation using culture containers comprising an elastic substrate of the invention; assisted zona hatching, for example, where the oocyte is mobilized or held in a container or pipet comprising an elastic substrate of the invention; transfer of gametes, a zygote, embryo, or blastocyst to the uterus or a fallopian tube, for example, using catheters and other transfer devices comprising an elastic substrate of the invention; preimplantation genetic diagnosis (PGD), for example, using pipets and containers comprising substrates of the invention; gamete intrafallopian transfer (GIFT) and zygote intrafallopian transfer (ZIFT) using pipets and containers comprising substrates of the invention; and the like as known in the art. Methods, compositions, and devices of the invention may be applicable to other ART procedures, and to variations on the ART procedures described herein, which vary depending on the state of the sperm and oocytes, geographic location, and other variables. The invention is also applicable to tools and techniques used in the transfer, culture, and manipulation of gametes and concepti during the generation of animals including transgenic animals, and cloning of animals.

Where cells are delivered to a recipient, e.g. in a transplantation method, the cells may be attached, encapsulated, embedded, etc. in a substrate of the invention. The substrate for such purposes is optionally biodegradable.

### Definitions

In the description that follows, a number of terms conventionally used in the field of cell culture are utilized extensively. In order to provide a clear and consistent understanding of the specification and claims, and the scope to be given to such terms, the following definitions are provided.

It is to be understood that this invention is not limited to the particular methodology, protocols, cell lines, animal species or genera, and reagents described, as such may vary. It is also to be understood that the terminology used herein is for the purpose of describing particular embodiments only, and is not intended to limit the scope of the present invention, which will be limited only by the appended claims.

As used herein the singular forms “a”, “an”, and “the” include plural referents unless the context clearly dictates otherwise. Thus, for example, reference to “a cell” includes a plurality of such cells and reference to “the culture” includes reference to one or more cultures and equivalents thereof known to those skilled in the art, and so forth. All technical and scientific terms used herein have the same meaning as commonly understood to one of ordinary skill in the art to which this invention belongs unless clearly indicated otherwise.

The term “cell culture” or “culture” means the maintenance of cells in an artificial, in vitro environment. Culture conditions may include, without limitation, a specifically dimensioned container, e.g. flask, roller bottle, plate, 96 well plate, etc.; culture medium comprising suitable factors and nutrients for growth of the desired cell type; and a substrate on the surface of the container or on particles suspended in the culture medium. By “container” is meant a glass, plastic, or metal vessel that can provide an aseptic environment for culturing cells.

A “long term culture” used herein refers to a culture in which stem cells grow, differentiate and are viable for at least a few hours, at least about 7 days, at least about 14 days, at least about 21 days, or at least about 28 days, or more.

The terms “primary culture” and “primary cells” refer to cells derived from intact or dissociated tissues or organs, or fragments thereof. A culture is considered primary until it is passaged (or subcultured) after which it is termed a “cell line” or a “cell strain.” The term “cell line” does not imply homogeneity or the degree to which a culture has been characterized. A cell line is termed “clonal cell line” or “clone” if it is derived from a single cell in a population of cultured cells. Primary cells can be obtained directly (or indirectly) from a human or animal or from adult or fetal tissue, including blood (e.g., cord blood). The primary cells may comprise a primary cell line, such as, but not limited to, a population of muscle satellite cells.

Substrate. As used herein, a substrate refers to a coating of a semi-solid matrix on a surface capable of being contacted by cells in a culture condition, where the surface may include, without limitation, the bottom of plates and flasks, etc., the inner wall of a roller bottle; or the surface of rods, particles, filaments and the like present within a culture container. For the methods of the present invention, a substrate is sufficiently thick that it masks the physical properties of the container, usually at least about 50 μm thick, at least about 100 μm thick, at least about 1000 μm. There are generally no adverse effects associated with increased thickness, but for convenience it may be desirable to have a substrate not more than about 10 mm in thickness. The substrate may be attached to a surface through adhesive, hydrophobic or hydrophilic interactions, gravity, etc.

Preferred substrates for the methods of the invention are hydrogels. The term “hydrogel” as used herein refers to a network of polymer chains that are water-insoluble, sometimes found as a colloidal gel in which water is the dispersion medium. Hydrogels can contain over 99% water and may comprise natural or synthetic polymers, or a combination thereof. In other instances, hydrogels may contain other percentages of water, as described herein. Hydrogels also possess a degree of flexibility due to their significant water content. A detailed description of suitable hydrogels may be found in published US patent application 20100055733, herein specifically incorporated by reference in its entirety for all purposes. The cell culture platform of the present disclosure may be fabricated from a soft and inert substrate that imbibes large amounts of water, thus approximating critical physicochemical aspects of the stem cell niche.

Containers and devices comprising one or more elastic substrates of the invention may include, without limitation, various shapes and sizes, e.g. Petri dish, centrifuge tubes, microcentrifuge tubes, cryovials, multi-well plates, T-flasks, beakers, test tubes, and the like. Devices coated with elastic substrates include, but are not limited to, holding pipets, and injection pipets. For example, the invention provides a method of holding an oocyte using a holding pipet coated with an elastic substrate, where the lumen of the holding pipet is blocked by a water-permeable elastic substrate preventing the oocyte from being drawn too far into the pipet. In another example, a holding pipet for immobilizing oocytes may be coated with elastic substrate with an open lumen. In another example, an injection pipet for use in ICSI may comprise a water-permeable elastic plug to contain the sperm within a defined region of the pipet to facilitate keeping track of the location of the sperm during handling.

Tissue Elasticity. In the methods of the invention, the elasticity of the substrate is selected to have physiological parameters, for example properties similar to the elasticity of the tissue from which the cell is derived or normally resident. Elasticity may be measured by any convenient method, as is known in the art. For example see, inter alia, Kaletunc et al. (1991) Food Hydrocolloids 5:237-247; Krall and Weitz (1998) Physical Review Letters 80:778-781; Melekaslan et al. (2003) Polymer Bulletin 50:287-294; each herein incorporated by reference. For example, a shear rheometer may be used to measure the elasticity of the substrate. In some cases the elastic moduli of these environments have not been measured, however it will be apparent to those familiar with measurements of biomechanical properties of tissues that it is currently possible to measure the elastic modulus of these structures, for example using rheological instruments or atomic force microscopy.

The term “mimics” or “approximates” the elasticity of the natural environment refers to an elastic substrate that provides for an elasticity that is general equivalent in range to the native environment of the cell or tissue in question, as discussed herein. The substrate of the invention may be within a range that is not more than 3 standard deviations from the range of the native environment, not more than 2 standard deviations from the range of the native environment, not more than 1 standard deviations from the range of the native environment. Alternatively the range may be not more than 2× different from the high or low value of the native environment; not more than 3× different from the high or low value of the native environment; not more than 5× different from the high or low value of the native environment; not more than 10× different from the high or low value of the native environment.

In some embodiments, the elasticity of the substrate will vary, depending on the specific somatic stem cell that is being cultured. For example, it is found the muscle stem cells are optimally grown on a substrate of at least about 1 kPa and not more than about 50 kPa, usually at least about 5 kPa and not more than about 25 kPa, and may be from about 7.5 to about 15 kPa. Similar elasticity may be utilized for stem cells residing in other soft tissue niches, e.g. neural stem cells, hematopoietic stem cells, liver stem cells, etc. Hard tissues, such as bone, may have a more rigid structure, e.g. at least about 100 kPa and up to as much as 10⁶ kPa. Aged and diseased tissues will typically have moduli of elasticity which are 0-80% higher than young or healthy tissue.

The elasticity range for a reproductive cell may be 0.01-80 kPa. The following elasticity ranges are appropriate for elastic substrates used to culture cells derived from, adapted to, or intended for the indicated tissue types: zona pellucida (0.5-50 kPa (Murayama, 2006; Khalilian, 2010; Sun, 2003)); endometrium (0.01-10 kPa); fallopian tube (0.01-10 kPa); uterus (5-40 kPa); vagina (3-15 kPa (Epstein, 2007)); testes (1-80 kPa); ovaries (0.1-80 kPa).

The elasticity range for a primary cell or cell line may be 0.001-250 kPa. The following elasticity ranges are appropriate for elastic substrates used to culture cells derived from, adapted to, or intended for the indicated tissue types: bone marrow (0.001-2 kPa; Winer, 2009); lens (10-150 kPa (Ziebarth, 2011)); brain (0.01-0.5 kPa (Ommaya, 1968)); cornea (130-250 kPa (Tanter, 2009)); bladder (0.1-10 kPa); spleen (0.1-10 kPa); small intestine (0.1-10 kPa); colon (0.01-10 kPa); rectum (0.01-10 kPa); lung (0.1-10 kPa); hair follicles (5-25 kPa (Pailler-Mattei et al. 2008)); pancreas (0.1-30 kPa); smooth muscle (2-100 kPa).

Elasticities of interest also include skeletal muscle (2-24 kPa (relaxed); 90-120 kPa (tensed) (Kwiatkowska et al. 2009)); kidney (1-5 kPa (Levental et al. 2007)); cardiac muscle (25-120 kPa (Mathur et al. 2001)); endothelium (1-7 kPa (Mathur et al. 2001)); liver (0.3-9 kPa); adipose tissue (5-100 Pa (Levental et al. 2007)) thyroid (5-15 kPa (Levental et al. 2007)); articular cartilage (0.5-1.5 MPa (Levental et al. 2007)); blood (<0.1 kPa (fluid)); skin (5-25 (Pailler-Mattei et al. 2008)); mammary fatty tissue (10-35 kPa (Krouskop et al. 1998)); mammary glandular tissue (14-49 kPa (Krouskop et al. 1998)); mammary gland (50-500 Pa (Levental et al. 2007)) prostate (30-70 kPa, (Krouskop et al. 1998)); lymph node (50-500 Pa).

The term “polymeric composition” as used herein refers to a single compound species or a mixture of compound species that may be cross-linked to form a polymer. Such precursor compounds include, but are not limited to, such as poly(ethylene glycol), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, alginate, protein polymers, methylcellulose and the like. The polymer compounds before polymerization may be toxic to, or otherwise inhibit the proliferation of a vertebrate cell, but it will be understood by those in the art that when polymerized, the polymer will be inert with respect to any cell or cell line in contact with the polymer.

In some embodiments of the invention, the hydrogel composition may comprise a polymer (or combination of polymers) selected from the group consisting of: a poly(ethylene glycol), a polyaliphatic polyurethane, a polyether polyurethane, a polyester polyurethane, a polyethylene copolymer, a polyamide, a polyvinyl alcohol, a polypropylene glycol, a polytetramethylene oxide, a polyvinyl pyrrolidone, a polyacrylamide, a poly(hydroxyethyl acrylate), a poly(hydroxyethyl methacrylate), poly(glycolic acid), poly(DL-lactic-co-glycolic acid), polyhydroxyalkanoate, poly(4-hydroxybutirate), sulfonated polymers, polygluconic acid, poly(acrylic acid), polyphosphazenes, polysaccharides, proteins, collagen, elastin, alginate, fibrin, fibronectin, laminin, hyaluronic acid, or another biologically-occurring polymer, polypeptide sequences cleavable by proteases including matrix metalloproteases, or copolymers formed from monomers of one or more of these.

The elasticity of the substrate may be influenced by a variety of factors, including, but not limited to, the branching of the polymer, the concentration of polymer, and the degree of cross-linking. For example, where the polymer is formed of polyethylene glycol vinyl sulfone (PEG-VS), the length of the PEG monomer and the branching, e.g. 2 arm, 4 arm, 8-arm, and the like may be varied to achieve the desired elasticity. In some embodiments, a non-swelling hydrogel is used.

Hydrogels of the invention optionally comprise at least one structural protein associated with a stem cell niche, e.g. fibronectin, laminin, collagen, and the like. Alternatively, or in combination, other proteins that have a beneficial or desired effect on the stem cells may be included in the hydrogel. Proteins may be conjugated to the hydrogel through a linker. The term “tether” or “linker” may refer to a molecular structure that conjugates a protein or polypeptide to the hydrogel. It is contemplated that a linker molecule suitable to link a biomolecule to the hydrogels of the disclosure can be, but is not limited to, a maleimide PEG-SVA linker; a dicarboxylic acid that further includes at least one available group, such as an amine group; for conjugating to a prosthetic group; and the like. It is also contemplated that other functional side groups may substitute for the amine group to allow for the linking to selected peptides. Exemplary dicarboxylic acids include, but are not limited to, aspartate, glutamate, and the like, and can have the general formula (HOOC)—(CH₂)ₙ—(CHNH₂⁺)—(CH₂)ₘ—(COOH), where n and m are each independently 0, or an integer from 1 to about 10. It is further considered within the scope of the disclosure for the linker to be a multimer, or a combination, of at least two such dicarboxylic acids. For example, such linker molecules may include, but are not limited to, (aspartate)ₓ, (glutamate)_{y}, or a combination thereof, where adjacent amino acids can be joined by peptide bonds, and the like. The subscripts x and y are each independently 0, or an integer from 1 to about 12.

Culture medium: The cells are grown in vitro in an appropriate liquid nutrient medium. Generally, the seeding level will be at least about 10 cells/ml, more usually at least about 100 cells/ml and generally not more than about 10⁵ cells/ml, usually not more than about 10⁴ cells/ml. Cells may be cultured singly or in groups.

Various media are commercially available and may be used, including, but not limited to, Ex vivo serum free medium, Dulbecco's Modified Eagle Medium (DMEM), RPMI, Iscove's medium, etc. The medium may be supplemented with serum or with defined additives. For example, a medium may include 5%, 10%, 15% serum, as known in the art. Appropriate antibiotics to prevent bacterial growth and other additives, including, but not limited to, pyruvate (0.1-5 mM), glutamine (0.5-5 mM), 2-mercaptoethanol (1-10×10⁻⁵ M) may also be included. The medium may be any conventional culture medium, generally supplemented with additives, including, but not limited to,iron-saturated transferrin, human serum albumin, soy bean lipids, linoleic acid, cholesterol, alpha thioglycerol, crystalline bovine hemin, etc., that allow for the growth of cells. In some circumstances, proliferative factors that do not induce cellular differentiation may be included in the cultures, e.g. c-kit ligand, LIF, and the like.

Stem cell: The term stem cell is used herein primarily to refer to a cell that has the ability both to self-renew, and to generate differentiated progeny (see Morrison et al. (1997) Cell 88:287-298). For example, the cell can be a mammalian or non-mammalian cell Mammals include laboratory models, e.g. rats, mice, rabbits, etc., farm animals and other domesticated animals such as horses, cats, pigs, dogs, sheep, etc.; and particularly include primates, more particularly humans. Generally, stem cells also have one or more of the following properties: an ability to undergo asynchronous, or asymmetric replication, that is where the two daughter cells after division can have different phenotypes; extensive self-renewal capacity; capacity for existence in a mitotically quiescent form; and clonal regeneration of all the tissue in which they exist, for example the ability of hematopoietic stem cells to reconstitute all hematopoietic lineages. “Progenitor cells” differ from stem cells in that they typically do not have the extensive self-renewal capacity, and often can only regenerate a subset of the lineages in the tissue from which they derive

Stem cells may be characterized by both the presence of markers associated with specific epitopes identified by antibodies and the absence of certain markers as identified by the lack of binding of specific antibodies. Stem cells may also be identified by functional assays both in vitro and in vivo, particularly assays relating to the ability of stem cells to give rise to multiple differentiated progeny. Stem cells include, but are not limited to, embryonic cells, certain germ cells, embryonic stem cells, induced pluripotent stem cells, multipotent stem cells, and the like, as well as somatic stem cells.

Somatic Stem cells: Somatic stem cells reside in differentiated tissue, but retain the properties of self-renewal and the ability to give rise to multiple cell types, usually cell types typical of the tissue in which the stem cells are found. Numerous examples of somatic stem cells are known to those of skill in the art, including, but not limited to, muscle stem cells (including without limitation satellite cells as described above), hematopoietic stem cells, neural stem cells, mesenchymal stem cells, pancreatic stem cells, hepatic stem cells, cardiac stem cells, kidney stem cells, liver stem cells.

Stem cells of interest include muscle stem cells, which may be evidenced by the ability to engraft and repopulate the myofiber-associated compartment in vivo following intramuscular injection, and subsequent maintenance of myogenic-colony forming capacity. Muscle cells include skeletal, cardiac and smooth muscles, but particularly skeletal muscle.

The term “muscle cell” as used herein refers to any cell which contributes to muscle tissue. Myoblasts, satellite cells, myotubes, and myofibril tissues are all included in the term “muscle cells”. Muscle cell effects may be induced within skeletal, cardiac and smooth muscles. Muscle tissue in adult vertebrates will regenerate from reserve myoblasts called “satellite cells”, or mesangioblasts, bone marrow derived cells, muscle interstitial cells, mesenchymal stem cells, etc. Satellite cells are distributed throughout muscle tissue and are mitotically quiescent in the absence of injury or disease. Following muscle injury or during recovery from disease, satellite cells will reenter the cell cycle, proliferate and 1) enter existing muscle fibers or 2) undergo differentiation into multinucleate myotubes, which form new muscle fiber. The myoblasts ultimately yield replacement muscle fibers or fuse into existing muscle fibers, thereby increasing fiber girth by the synthesis of contractile apparatus components. This process is illustrated, for example, by the nearly complete regeneration which occurs in mammals following induced muscle fiber degeneration; the muscle progenitor cells proliferate and fuse together regenerating muscle fibers.

One example of muscle stem cells is cells characterized as CD45⁻, CD11b⁻, CD31⁻, Sca1⁻, α7 integrin⁺, and CD34⁺. In some embodiments, muscle stem cells cultured on the elastic substrate described herein may be implanted into a recipient subject mammal, wherein the cells or population of cells differentiate into muscle cells, e.g. for regeneration.

Regeneration as used herein may refer to the process by which new cells form from progenitor cells. Progenitor cells include, but are not limited to, stem cells, multipotent cells, pluripotent cells, primary cells or cell lines, reprogrammed cells, transdifferentiated cells, dedifferentiated or induced pluripotent stem cells. Such cells may be manipulated ex vivo and transplanted to a recipient for regeneration, where the cells can be delivered in a suitable medium; contained in a substrate of the invention, etc.

Muscle regeneration as used herein refers to the process by which new muscle fibers form from muscle progenitor cells. A therapeutic composition will usually confer an increase in the number of new fibers by at least 1%, more preferably by at least 20%, and most preferably by at least 50%. The growth of muscle may occur by the increase in the fiber size and/or by increasing the number of fibers. The growth of muscle may be measured by an increase in wet weight, an increase in protein content, an increase in the number of muscle fibers, an increase in muscle fiber diameter; etc. An increase in growth of a muscle fiber can be defined as an increase in the diameter where the diameter is defined as the minor axis of ellipsis of the cross section.

Muscle regeneration may also be monitored by the mitotic index of muscle. For example, cells may be exposed to a labeling agent for a time equivalent to two doubling times. The mitotic index is the fraction of cells in the culture which have labeled nuclei when grown in the presence of a tracer which only incorporates during S phase (i.e., BrdU) and the doubling time is defined as the average S time required for the number of cells in the culture to increase by a factor of two. Alternatively, activation in vivo may be detected by monitoring the appearance of the intermediate filament vimentin by immunological or RNA analysis methods. When vimentin is assayed, a useful activator may cause expression of detectable levels of vimentin in the muscle tissue when the therapeutically useful dosage is provided. Productive muscle regeneration may be also monitored by an increase in muscle strength and agility.

Muscle regeneration may also be measured by quantitation of myogenesis, i.e. fusion of myoblasts to yield myotubes. An effect on myogenesis results in an increase in the fusion of myoblasts and the enablement of the muscle differentiation program. For example, the myogenesis may be measured by the fraction of nuclei present in multinucleated cells in relative to the total number of nuclei present. Myogenesis may also be determined by assaying the number of nuclei per area in myotubes or by measurement of the levels of muscle specific protein by Western analysis.

The survival of muscle fibers may refer to the prevention of loss of muscle fibers as evidenced by necrosis or apoptosis or the prevention of other mechanisms of muscle fiber loss. Muscles can be lost from injury, atrophy, and the like, where atrophy of muscle refers to a significant loss in muscle fiber girth.

The terms “grafting”, “engrafting”, and “transplanting” and “graft” and “transplantation” as used herein refer to the process by which stem cells or other cells according to the present disclosure are delivered to the site where the cells are intended to exhibit an effect, such as, but not limited to, repairing damage to a patient's central nervous system, treating autoimmune diseases, treating diabetes, treating neurodegenerative diseases, or treating the effects of nerve, muscle and/or other damage caused by birth defects, stroke, cardiovascular disease, a heart attack or physical injury or trauma or genetic damage or environmental insult to the body, caused by, for example, disease, an accident or other activity. The stem cells or other cells for use in the methods of the present disclosure can also be delivered in a remote area of the body by any mode of administration as described herein. For example, the term “cell engraftment” as used herein can refer to the process by which cells such as, but not limited to, muscle stem cells, are delivered to, and become incorporated into, a differentiated tissue such as a muscle, and become a part of that tissue. For example, muscle stem cells, when delivered to a muscle tissue, may proliferate as stem cells, and/or may bind to skeletal muscle tissue, differentiate into functional myoblasts cells, and subsequently develop into functioning myofibers. Transplantation may utilize a dose of cells effective to obtain the desired effect, which may be delivered in an appropriate medium or substrate, including an elastic substrate of the invention.

Stem cell environment. The tissue in which stem cells are normally resident may be referred to as a native microenvironment, or niche. Typically the in vivo environment will include physical properties, e.g. elasticity, fluid flow, fibrous proteins that contact the cells, etc.; and may further include biochemical factors that interact with stem cells to regulate stem cell fate. In many cases, the localization (locale) of the niche within the tissue is known, although not always. In most cases, the precise components of stem cell niches remain unknown. Within adult tissue, stem cell niches maintain adult stem cells in a typically quiescent state, but after tissue injury, the surrounding micro-environment actively produces signals to promote self renewal or differentiation to form new tissues, the process of regeneration. Niche variables include, but are not limited to, cell-cell interactions between stem cells; interactions between stem cells and neighbouring differentiated cells; interactions between stem cells and adhesion molecules; extracellular matrix components; oxygen tension; growth factors; cytokines, and physiochemical nature of the environment including the pH, etc. The physical parameters of the niche with respect to substrate elasticity may be determined empirically, or may be determined from published references (for example see Engler et al. (2006) Cell 126:677).

Within a niche, stem cells are frequently anchored to a basal lamina or stromal cells that can provide a substrate for oriented cell division. The basal lamina is a regulator of the accessibility of growth factors and other signals, as associated extracellular matrix (ECM) molecules and glycoproteins can both concentrate and sequester factors in inactive or active forms. Cell anchoring may orient cell division resulting in the segregation of key determinants into one or both daughter cells depending on the plane of division.

Hematopoietic stem cell niche. Vertebrate hematopoietic stem cells' niche is in the bone marrow formed by cells subendoosteal osteoblasts, sinusoidal endothelial cells, vascular cells, and bone marrow stromal (also sometimes called reticular) cells which includes a mix of fibroblastoid, monocytic and adipocytic cells.

Hair follicle stem cell niche. The bulge area at the junction of arrectores pili muscle to the hair follicle sheath has been shown to host the skin stem cells with maximum span of developmental potential. There cells are maintained by signaling in concert with niche cells—signals include paracrine (e.g. sonic hedgehog), autocrine and juxtacrine signals.

Intestinal stem cell niche. The subepithelial fibroblast/myofibroblast network which surround the intestinal crypts constitute the niche.

Neural stem cell niche. Neurogenesis occurs in two principal brain regions in adult mammals: the subventricular zone (SVZ), adjacent to the lateral ventricles, and the subgranular zone (SGZ) of the hippocampal formation. The SVZ consists of a thin layer of dividing cells that extends along the length of the lateral walls of the lateral ventricles and is largely separated from the cerebrospinal fluid (CSF) by a layer of multi-ciliated ependymal cells. Newly generated neuroblasts traverse a network of chains which extends throughout the SVZ to join the rostral migratory stream (RMS) that leads to the olfactory bulb. There they differentiate into two kinds of inhibitory interneurons, granule and periglomerular cells, and functionally integrate into the existing circuitry. The SGZ is located between the hilus and the granule cell layer of the dentate gyrus. Newly generated granule neurons born in the SGZ migrate only a short distance to the granule cell layer, where they extend dendrites to the molecular layer and an axon along the mossy fibre path and integrate functionally into the circuitry of the dentate gyrus. In both regions, a subset of astrocytes, glial cells classically associated with support functions in the brain, are the in vivo primary precursors for adult neurogenesis. These cells have been defined as astrocytes based on their ultrastructural features, markers they express and electrophysiological properties.

Muscle stem cell niche. Muscle stem cells are typically sandwiched between the basement membrane and sarcolemma (cell membrane) of individual muscle fibres, and can be difficult to distinguish from the sub-sarcolemmal nuclei of the fibres. These cells are able to differentiate and fuse to augment existing muscle fibres and to form new fibres. (see Boonen and Post (2008) Tissue Eng Part B Rev. 14(4):419-31; Cosgrove and Blau (2010) Differentiation Review; and Lutolf, Gilbert, Blau (2010) Nature Review.

The term “candidate cells” refers to any type of cell that can be placed in culture described herein, including cells which may have the potential to be used to regenerate tissues, for example, but the term “candidate cells” is not limited to stem cells. Candidate cells include without limitations, mixed cell populations, ES cells and progeny thereof, e.g. embryoid bodies, embryoid-like bodies, embryonic germ cells, stem cells, reprogrammed cells, transdifferentiated cells, dedifferentiated cells, induced pluripotent cells (iPS), multipotent cells, pluripotent cells, or primary cells or cell lines.

The term “candidate agents” means oligonucleotides, polynucleotides, siRNA, shRNA genes, gene products, small molecules, peptides, polypeptides, proteins, antibodies, or fragments thereof, or pharmacological compounds that are introduced in a cell culture described herein to assay for their effect on the explants.

The term “contacting” refers to the placing of either candidate cells or candidate agents in the cell culture of. Contacting also encompasses co-culturing candidate cells with cultured cells for a period of time sufficient to observe the parameters of interest.

“Screening” refers to the process of either culturing candidate cells with or adding candidate agents to the cell culture described herein. For example, the effect of the candidate cells or candidate agents is assessed by an increase in expansion of cells (e.g., stem cells, or other cell described herein) over basal levels and by the presence of multilineage differentiation markers indicative of such cells. The effect of candidate cells or candidate agents can be further evaluated by assaying the cells for long-term reconstitutive activity by serial in vitro passage, as well as by in vivo transplantation.

The term “proliferative status” as used herein refers to whether a population of cells or a subpopulation thereof, are dividing and thereby increasing in number, in the quiescent state, or whether the cells are not proliferating, dying or undergoing apoptosis. For example, the population of cells may be hematopoietic stem or progenitor cells, or a subpopulation thereof.

The terms “modulating the proliferative status” or “modulating the proliferation” as used herein refers to the ability of a compound to alter the proliferation rate of a population of cells. A compound may be toxic, wherein the proliferation of the cells is slowed or halted, or the proliferation may be enhanced such as, for example, by the addition to the cells of a cytokine or growth factor.

The term “quiescent” as used herein refers to cells that are not actively proliferating by means of the mitotic cell cycle. Quiescent cells (which include cells in which quiescence has been induced as well as those cells which are naturally quiescent, such as certain fully differentiated cells) are generally regarded as not being in any of the four phases G1, S, G2 and M of the cell cycle; they are usually described as being in a G0 state, so as to indicate that they would not normally progress through the cycle. Cultured cells can be induced to enter the quiescent state by various methods including, but not limited to, chemical treatments, nutrient deprivation, growth inhibition or manipulation of gene expression, and induced to exit the quiescent state by contacting the cells with a compound, such as a cytokine or growth factor.

Conventional techniques can be utilized to assay cell function, or the algorithms for cell tracking described herein. Due to the transparency of the hydrogel, cells can be studied by live time-lapse microscopy, using bright field or fluorescence. Live cells can be removed from desired wells by micromanipulation for subsequent experiments. They can be fixed and immunostained after cell culture for retrospective phenotypic analyses. Platforms can be generated that are well suited to look at rare events of single cells such as asymmetric stem cell division. On the other hand, platforms that are engineered for high-throughput drug screening purposes on a chip can be fabricated. The hydrogels of the invention are defined in composition and therefore the numbers and types of proteins and chemicals to which the cells are exposed can be defined.

The terms “system” and “computer-based system” refer to the hardware means, software means, and data storage means used to analyze the information of the present disclosure. The minimum hardware of the computer-based systems of the present invention comprises a central processing unit (CPU), input means, output means, and data storage means. As such, any convenient computer-based system may be employed in the present disclosure. The data storage means may comprise any manufacture comprising a recording of the present information as described above, or a memory access means that can access such a manufacture.

A “processor” references any hardware and/or software combination which will perform the functions required of it. For example, any processor herein may be a programmable digital microprocessor such as available in the form of an electronic controller, mainframe, server or personal computer (desktop or portable). Where the processor is programmable, suitable programming can be communicated from a remote location to the processor, or previously saved in a computer program product (such as a portable or fixed computer readable storage medium, whether magnetic, optical or solid state device based). For example, a magnetic medium or optical disk may carry the programming, and can be read by a suitable reader communicating with each processor at its corresponding station.

“Computer readable medium” as used herein refers to any storage or transmission medium that participates in providing instructions and/or data to a computer for execution and/or processing. Examples of storage media include floppy disks, magnetic tape, USB, CD-ROM, a hard disk drive, a ROM or integrated circuit, a magneto-optical disk, or a computer readable card such as a PCMCIA card and the like, whether or not such devices are internal or external to the computer. A file containing information may be “stored” on computer readable medium, where “storing” means recording information such that it is accessible and retrievable at a later date by a computer. A file may be stored in permanent memory.

With respect to computer readable media, “permanent memory” refers to memory that is permanently stored on a data storage medium. Permanent memory is not erased by termination of the electrical supply to a computer or processor. Computer hard-drive ROM (i.e. ROM not used as virtual memory), CD-ROM, floppy disk and DVD are all examples of permanent memory. Random Access Memory (RAM) is an example of non-permanent memory. A file in permanent memory may be editable and re-writable.

To “record” data, programming or other information on a computer readable medium refers to a process for storing information, using any convenient method. Any convenient data storage structure may be chosen, based on the means used to access the stored information. A variety of data processor programs and formats can be used for storage, e.g. word processing text file, database format, etc.

A “memory” or “memory unit” refers to any device which can store information for subsequent retrieval by a processor, and may include magnetic or optical devices (such as a hard disk, floppy disk, CD, or DVD), or solid state memory devices (such as volatile or non-volatile RAM). A memory or memory unit may have more than one physical memory device of the same or different types (for example, a memory may have multiple memory devices such as multiple hard drives or multiple solid state memory devices or some combination of hard drives and solid state memory devices).

A system can include hardware components which take the form of one or more platforms, e.g., in the form of servers, such that any functional elements of the system, i.e., those elements of the system that carry out specific tasks (such as managing input and output of information, processing information, etc.) of the system may be carried out by the execution of software applications on and across the one or more computer platforms represented of the system. The one or more platforms present in the subject systems may be any convenient type of computer platform, e.g., such as a server, main-frame computer, a work station, etc. Where more than one platform is present, the platforms may be connected via any convenient type of connection, e.g., cabling or other communication system including wireless systems, either networked or otherwise. Where more than one platform is present, the platforms may be co-located or they may be physically separated. Various operating systems may be employed on any of the computer platforms, where representative operating systems include Windows, MacOS, Sun Solaris, Linux, OS/400, Compaq Tru64 Unix, SGI IRIX, Siemens Reliant Unix, and others. The functional elements of system may also be implemented in accordance with a variety of software facilitators, platforms, or other convenient method.

Items of data are “linked” to one another in a memory when the same data input (for example, filename or directory name or search term) retrieves the linked items (in a same file or not) or an input of one or more of the linked items retrieves one or more of the others.

Subject computer readable media may be at a “remote location”, where “remote location,” means a location other than the location at which the x-ray crystallographic or other analysis is carried out. For example, a remote location could be another location (e.g., office, lab, etc.) in the same city, another location in a different city, another location in a different state, another location in a different country, etc. As such, when one item is indicated as being “remote” from another, what is meant is that the two items may be in the same room but separated, or at least in different rooms or different buildings, and may be at least one mile, ten miles, or at least one hundred miles apart.

“Communicating” information references transmitting the data representing that information as, e.g., electrical or optical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. Examples of communicating media include radio or infra-red transmission channels as well as a network connection to another computer or networked device, and the Internet or Intranets including email transmissions and information recorded on websites and the like.

Acrosome: a compartment of the sperm which contains molecules which enable the sperm to penetrate the zona pellucida surrounding the oocyte. Acrosome reaction: the release from the sperm of molecules which allow it to penetrate the zona pellucida surrounding the oocyte.

Assisted zona hatching (AZH): The use of chemical (acid), mechanical (knife or needle), or laser methods to create a small hole in the zona pellucida to increase the probability of the blastocyst hatching from the zona pellucida and thus being able to implant into the endometrium. AZH is usually performed shortly before embryo transfer into the uterus.

Blastocyst: A thin-walled hollow structure consisting of approximately 150 cells, comprising an inner cell mass, an outer layer (the trophoblast), and a fluid-filled cavity (the blastocoel). The blastocyst forms around day 5 after fertilization. Transfer of the blastocyst into the uterus increases the chance of fertilization, compared to transfer of earlier stage embryos, possibly because earlier stage embryos typically are found in the fallopian tubes rather than the uterus, where embyros or blastocysts are often deposited during ART procedures. Blastocyst cavity is the liquid-filled volume within the blastocyst, surrounded on all sides by the spherical shell of trophoblast cells and partially bordered by the inner cell mass. The formation of the blastocyst cavity may be referred to as cavitation. Blastomere is a cell of the pre-blastocyst embryo.

Capacitation: Biochemical changes in the sperm which facilitate fertilization. These changes are induced by molecules in the semen and female reproductive tract which cause the sperm membrane to become more fluid, the sperm to become more mobile, and the sperm acrosome head to destabilize facilitating binding of the sperm to the zona pellucida.

Cleavage: A series of rapid mitotic divisions of the zygote into roughly equal-volume cells. The first cleavage, of the zygote to form the 2-cell preembryo, occurs approximately 30 hours after fertilization. Subsequent cleavages occur approximately 10-12 hours apart. The divisions occur approximately synchronously in the cells but the exact timing of the divisions varies.

Conceptus: The oocyte after it has been penetrated by a sperm, or any of its derivatives from this stage until birth, including the sperm-penetrated oocyte, fertilized oocyte, prezygote, ootid, zygote, pre-embryo, embryo, morula, early blastocyst, blastocyst, hatched blastocyst, and including the structures which surround these derivatives, including the perivitelline space, the zona pellucida, the corona radiata, and the cumulus oophorous. The term may be used herein to refer to the derivatives of the oocyte at all stages of development up until birth, including the penetrated oocyte, fertilized oocyte, pre-zygote, zygote, pre-embryo, embryo, morula, early blastocyst, and late blastocyst. The term “oocyte” may be used to refer to either the oocyte or cumulus-oocyte complex.

Corona radiata (CR): The CR comprises the innermost two or three layers of cumulus cells surrounding the zona pellucida. The cumulus cells are embedded in a gel consisting largely of hyaluronic acid. To reach the zona pellucida sperm secrete hyaluronidase which digests the hyaluronic acid. Many sperm are required to secrete enough hyaluronidase to weaken the corona radiata sufficiently for a single sperm to penetrate to the zona pellucida.

Cortical reaction: Within a minute of a sperm fusing with the plasma membrane of an oocyte, cortical granules are released from the oocyte into the perivitelline space. The granules contain proteases which cleave bonds in the zona pellucida, allowing other bonds to form, causing the zona pellucida to become mechanically stiffer and more resistant to digestion, both of which processes are referred to as hardening.

Cumulus cells: Cells of the cumulus oophorous embedded in a gel comprising hyaluronic acid. Cumulus cells have branching cone-shaped processes which penetrate into the zona pellucida and contact microvilli of the oocyte during its development in the follicle. Cumulus oophorus (CO): A mass of cells surrounding the egg and zona pellucida, embedded in a gel comprising hyaluronic acid, and indeed the cumulus oophorous can be stripped from the oocyte and zona pellucida by digestion with hyaluronidase. The inner layer of the CO is the corona radiata. The CO surrounds the egg and zona pellucida starting in the follicle and as long as 72 hours after fertilization. The cumulus-conceptus complex (CCC) is the assembly comprising the perivitelline space, zona pellucida, corona radiata, cumulus oophorous, and the cells resulting from the fertilization of the oocyte, including the prezygote, zygote, and embryo cells. Herein the CCC is used interchangeably with cumulus-oocyte complex (COC), defined below.

Cumulus stripping: The cumulus cells are mechanically removed from the zona pellucida and oocyte, typically using shear force resulting from uptake into and expulsion from a stripping pipet. Stripping is performed before ICSI to allow easy access to the egg. If traditional insemination by mixing sperm and egg is performed, stripping is often not performed because the cumulus cells are necessary for causing the sperm to properly penetrate the zona pellucida and fuse with the plasma membrane of the egg.

Cytoplasmic transfer: The injection of the cytoplasm of one cell into another cell, for example the injection of the cytoplasm of a normally fertile egg into an egg with impaired fertility to rescue its fertility.

Egg: The female gamete or gametocyte. At the time of release from the ovary the egg has not yet undergone its final meiotic division, and thus has 46 chromosomes. After fertilization, the nucleus divides to form a pronucleus with 23 chromosomes and a (third) polar body which is ejected from the egg and can be seen in the perivitelline space, between the egg and the zona pellucida.

Embryo: The developing organism, starting from the time the zygote divides, up until the fetal stage, which in humans begins at the start of the 9th week after fertilization. The developmental processes from the time of fertilization of the secondary oocyte up until the end of the embryo stage, which in humans is the end of the eighth week after fertilization, may be referred to as embryogenesis.

| | | Stage of gamete or | |
|---|---|---|---|
| Day | Event | conceptus | Description |
| Before | Maturation of | Secondary oocyte | The secondary oocyte is |
| E01 | the oocyte in | | surrounded by the fluid |
| | the follicle | | of the PS, the ZP, and |
| | | | the CO. |
| E0 | Ovulation | Secondary oocyte | The COC emerges from |
| | | | the ovarian follicle. |
| E0.5 | Fertilization, | Penetrated oocyte | The zona pellucida |
| | completion | (or prezygote) | hardens; the perivitelline |
| | of meiosis | | space enlarges. |
| E1 | Zygote | Zygote | The pronuclei |
| | formation | | membranes dissolve. |
| E1.5 | Cleavage | 2-cell embryo | Zona pellucida softens |
| E2 | ″ | 4-cell embryo | and thins |
| E2.5 | ″ | 8-cell embryo | |
| E3.0 | Compaction | 16-32 cell embryo- | |
| | | morula | |
| E4.0 | Cavitation | Early blastocyst | |
| E4-5 | Zona | | Blastocyst is free of |
| | hatching | | zona pellucida |
| E5-7 | Implantation | Late blastocyst | Blastocyst attaches to |
| | | | epithelials cells lining |
| | | | a crypt of the |
| | | | endometerium |

Embryo splitting: Removing a cell or cells from an embryo and culturing them as separate embryos, to produce more embryos.

Fertilized egg: The egg for approximately 12 hours after it fuses with the sperm. The fertilized egg becomes the zygote when the male and female pronuclei fuse. Typically within another 12 hours the zygote cleaves becoming the 2-cell embryo.

Fimbriae: Finger-like structures projecting from the end of the fallopian tube. At the time of ovulation sex hormones cause the fimbriae to swell, and some of them brush against the ovary, allowing them to sweep the newly hatched egg into the infundibulum.

Gamete: A sperm (spermatozoan) or egg (oocyte), at any stage of development up to the penetration of the oocyte by the sperm, at which time the conceptus is formed. Herein, the term gamete includes both bare oocytes and cumulus-oocyte-complexes. Gamete intrafallopian transfer (GIFT) is the placement of oocytes and sperm into the fallopian tubes.

Inner cell mass (ICM): A group of blastocyst cells within the surrounding layer of trophoblast cells. The ICM gives rise to the fetus, while the trophoblast gives rise to the much of the placenta.

In vitro fertilization (IVF): IVF is the placement of sperm near or in an egg to cause fertilization. IVF is often used to refer to the entire procedure of egg harvesting, egg fertilization, embryo culture, and embryo transfer to the uterus.

Oocyte: The female gamete at any stage from the start of the first meiotic division until fertilization. Herein, oocyte can also refer to the cumulus-oocyte complex (COC).

Perivitelline space: A fine slit between the oocyte and the zona pellucida, which may enlarge during the cortical reaction following fertilization, thereby helping to prevent polyspermic fertilization.

Preembryo: The conceptus after the first cleavage of the zygote approximately 30 hours after fertilization, up until the formation of the blastocyst.

Preimplantation genetic diagnosis (PGD): Genetic screening of a cell or cells removed from an embryo, using, for example, Comparative Genomic Hybridization (CGH) or Fluorescent In Situ Hybridization (FISH).

Prezygote: The sperm-penetrated oocyte before breakdown of the pronuclear membranes.

Pronuclei: Membrane-bound bodies containing either the male or female DNA within the sperm-penetrated oocyte, or pre-zygote.

Secondary oocyte: The female gamete before fertilization. The secondary oocyte is a haploid cell resulting from meiosis I of a primary oocyte, and halted at metaphase II. This means that in humans the secondary oocyte has 23 chromosomes, and each chromosome has undergone synthesis of a sister chromatid strand joined at the centromere. Upon penetration by a sperm the secondary oocyte completes meiosis II and ejects a polar body into the perivetelline space and becomes a prezygote or sperm-penetrated oocyte. In humans the secondary oocyte is approximately 100-150 microns in diameter at the time of fertilization.

Sperm: The male gamete, gametocyte, spermatocyte, also called a spermatozoon. Sperm are not capable of fertilization when they first enter the vagina. They must be exposed to the acidic environment of the vagina for several hours, resulting in an increase in their motility, a process called sperm activation.

Intracytoplasmic sperm injection (SICI): The injection of a sperm into the cytoplasm of an oocyte. This is typically accomplished by first stripping the cumulus cells from the oocyte, then holding the oocyte with a holding pipet while the sperm is delivered through a much finer, sharper pipet.

Sperm activation: Increase in activity level of sperm resulting from several hours spent in the female reproductive tract or certain artificial conditions in vitro.

Transvaginal ovum retrieval (OCR): The surgical procedure by which a needle is introduced to the ovary through the vaginal wall to retrieve eggs which were released in response to the procedure of ovarian stimulation. The procedure takes about a quarter hour and the patient is usually under general anesthetic.

Zona hatching: The exit of the blastocyst from the zona pellucida. In humans this typically occurs approximately 5-6 days after fertilization.

Zona pellucida: A layer of glycoproteins surrounding the plasma membrane of the secondary oocyte, ovum, zygote, embryo, morula, and early blastocyst, until the blastocyst hatches from the zona pellucida approximately 5-6 days after fertilization. Before fertilization, the zona pellucida binds sperm causing the acrosome reaction to occur, by which the sperm exposes enzymes to the zona pellucida, resulting in a hole of approximately the size of the sperm head being created in the zona pellucida. After the sperm fuses with the membrane of the secondary oocyte, which is fertilization, the oocyte undergoes the cortical reaction, which causes cortical granules to be released from the oocyte into the perivitelline space. The granules contain proteases which cleave bonds in the zona pellucida, allowing other bonds to form, causing the zona pellucida to become mechanically stiffer and more resistant to digestion.

Zona reaction: Within five minutes of sperm fusion with the plasma membrane of the oocyte, the zona pellucida undergoes changes which mechanically harden it and prevent further sperm binding to it. The zona reaction is caused by molecules released from cortical granules by the oocyte.

Zygote: A fertilized oocyte after the breakdown of the membranes of the male and female pronuclei, which typically takes place within approximately 12 hours of fertilization, and before the first cleavage. After the first cleavage the zygote becomes an embryo. Before the breakdown of the pronuclei membranes, the fertilized oocyte is called a prezygote. Zygote intrafallopian transfer (ZIFT) is the placing of fertilized oocytes into the fallopian tube.

### DETAILED DESCRIPTION

### Cells

The present disclosure provides methods and compositions for culturing cells. In some cases, the cell is a primary cell, stem cell, transdifferentiated cell, dedifferentiated cell, reprogrammed cell, multipotent cell, or pluripotent cell. In some cases, the cell is a stem cell; in other cases, the cell is not a stem cell. The cell can be any cell of the body, including, but not limited to: muscle cell, hematopoietic cell, lymphocyte, mononuclear cell, neural cell, mesenchymal cell, pancreatic cell, hepatic cell, heart cell, kidney cell, liver cell, skeletal muscle cell, mammary cell, mammary gland cell, endothelial cell, adipose tissue cell, thyroid cell, articular cartilage, skin cell, prostate cell, blood cell, retinal cell, dental pulp, bladder cell, spleen cell, small intestine cell, colon, rectal cell, lung, hair follicles, intestinal cell, or bone marrow.

The cells are preferably human but can also be non-human, e.g., non-human mammals. Examples of non-human mammals include, but are not limited to, non-human primates (e.g., apes, monkeys, gorillas), rodents (e.g., mice, rats), cows, pigs, sheep, horses, dogs, cats, or rabbits. Similarly, the cell can be from any organism, reptile, microbe, or microorganism. Often, the cells are derived from a human subject or human patient. The subject may be free of a disease or disorder, or the subject may suffer from a disease or disorder, or at risk for such disease or disorder. Examples of diseases are provided herein. The subject may be a female; in some cases, the subject is a male. In some cases, the subject is a female over, or under, the age of 20, 25, 30, 35, 40, 41, 42, 43, 44, 45, 50, or 55. In some cases, the subject is a male over, or under, the age of 20, 25, 30, 35, 40,41, 42, 43, 44, 45, 50, or 55.

As described herein, in certain embodiments, the cell is a stem cell. A stem cell includes, but is not limited to, an embryonic stem cell, a somatic stem cell, an induced pluripotent stem cell, a multipotent stem cell, or certain germ cells (e.g., gametocyte). In some cases, the somatic stem cell is a muscle cell, hematopoietic cell, lymphocyte, mononuclear cell, neural cell, mesenchymal cell, pancreatic cell, hepatic cell, heart cell, kidney cell, liver cell, skeletal muscle cell, mammary cell, mammary gland cell, endothelial cell, adipose tissue cell, thyroid cell, articular cartilage, skin cell, prostate cell, blood cell, retinal cell, dental pulp, bladder cell, spleen cell, small intestine cell, colon, rectal cell, lung, hair follicles, intestinal cell, or bone marrow.

A germ cell may be a gametocyte, gamete, oocyte, spermatocyte, or reproductive cell. An induced stem cell (e.g, induced pluripotent stem cell) may be derived from a somatic cell such as a muscle cell, hematopoietic cell, lymphocyte, mononuclear cell, neural cell, mesenchymal cell, pancreatic cell, hepatic cell, heart cell, kidney cell, liver cell, skeletal muscle cell, mammary cell, mammary gland cell, endothelial cell, adipose tissue cell, thyroid cell, articular cartilage, skin cell, prostate cell, blood cell, retinal cell, dental pulp, bladder cell, spleen cell; small intestine cell, colon, rectal cell, lung, hair follicles, intestinal cell, or bone marrow. In some cases, the cells are derived from reproductive tissue (e.g., fallopian tube, endometrium, uterine, ovarian), fetal tissue, or cord blood.

### Expansion and Other Manipulations of Stem Cells

A population of cells comprising stem cells is cultured in vitro on a substrate with a defined elasticity as described herein. The cells are maintained in culture for a period of time sufficient to increase the number of stem cells (e.g., assayable stem cells) in the culture, or to perform necessary manipulations (e.g., genetic modification; exposure to drugs).

For expansion, the number of assayable stem cells may be demonstrated by a number of assays appropriate to the specific type of stem cell, as described herein. Following the initial seeding, there is an expansion, where the number of assayable stem cells having the functional phenotype of the initial cell population can increase from about 2, about 5, to about 100 fold or more. The cells can be frozen using conventional methods at any time, usually after the first week of culture. After seeding the culture medium, the culture medium is maintained under conventional conditions for growth of mammalian cells, generally about 37° C. and 5% CO₂ in 100% humidified atmosphere. Fresh media may be conveniently replaced, in part, by removing a portion of the media and replacing it with fresh media. Various commercially available systems have been developed for the growth of mammalian cells to provide for removal of adverse metabolic products, replenishment of nutrients, and maintenance of oxygen. By employing these systems, the medium may be maintained as a continuous medium, so that the concentrations of the various ingredients are maintained relatively constant or within a predescribed range. Such systems can provide for enhanced maintenance and growth of the subject cells using the designated media and additives.

In some embodiments, the invention provides a method for manipulation of stem cells, for example maintenance, transfer and/or expansion by performing such manipulations in a container or device comprising a substrate with an elasticity that mimics the elasticity of the in vivo microenvironment of the cell. Cells of interest include, without limitation, skeletal muscle (2-24 kPa (relaxed); 90-120 kPa (tensed); mammary fatty tissue (10-35 kPa); mammary glandular tissue (14-49 kPa); mammary gland (50-500 Pa); kidney (1-5 kPa); cardiac muscle (25-120 kPa); endothelium (1-7 kPa); liver (0.3-9 kPa); adipose tissue (5-100 Pa); thyroid (5-15 kPa); articular cartilage (0.5-1.5 MPa); skin (5-25); prostate (30-70 kPa); lymph node (50-500 Pa); blood (<0.1 kPa); retina; dental pulp; bladder; spleen; small intestine; colon; rectum; lung; hair follicles; pancreas; smooth muscle; intestine; bone marrow; heart; germ cells; reproductive cells; and also include co-cultures with other cells of interest.

Manipulations may include, but are not limited to, genetic modification; epigenetic modification; fusion with another cell or cells; adaptation of the cells to an elasticity; adaptation of the cells to a chemical stimulus; dedifferentiation; transdifferentiation; change in expression level of one or more genes; change in state of signaling pathways or metabolic pathways; change in cell elasticity; extension of telomeres; freezing, storage, or thawing; rejuvenation of one or more cellular functions; rejuvenation of one or more cellular structures or components; repair or replacement of DNA; repair or replacement of mitochondria; repair or replacement of cellular components; removal of waste products; covalent modification of cellular proteins, lipids, or carbohydrates; exposure to a drug; exposure to a cell; exposure to a polypeptide; in vitro fertilization; maturation of a fertilized oocyte to embryo or blastocyst stage; expanding the number of cells. In some embodiments provided herein, such manipulations are performed on cells that are not necessarily stem cells, such as certain primary cells, germ cells, differentiated cells, terminally differentiated cells, somatic cells, etc.

### Cell Culture Substrate

This disclosure provides cell culture substrates for use in the culture of cells, including somatic cells and cell lines, as well as stem cells, reproductive cells, etc. In some instances, the cell culture substrate is a hydrogel, a cell-free scaffold, a foam rubber, a soft plastic, a gel, a putty, an aerogel, a fabric, a paste, an oil, or a wax. In some cases, the cell culture substrate is primarily made up of water. The cell culture substrate may further comprise one or more channels through the substrate or wells on top of the substrate. For example, a cell may be placed on top of a channel or inside of a well on the cell culture substrate. The channels through the cell culture substrate may also allow perfusion of solutions through the cell culture substrate. The channels may allow the perfusion of an oxygenated cell culture media through the cell culture substrate. In some cases, a mold may be placed in contact with the precursor solution while it is curing, and then removed from the cell culture substrate. For example, the cell culture substrate or substrates may be fabricated outside of the container and then placed in the container. The cell culture substrate may also be attached covalently or non-covalently to the container, for example by an adhesive. Elastic substrates may be formed in a variety of shapes, e.g. cilia, folds, crypts, striations, processes, dimples, partial spherical shells, and bumps, which optionally mimic a native environment of the cell. The substrate may also be subjected to twisting, compressing, stretching, or otherwise deforming the elastic substrate during culture.

A cell culture substrate may be a hydrogel comprising a polymer selected from a group comprising polyethylene glycol, poly(vinyl acetate), poly(ethylene acrylate), polyaliphatic polyurethane, polyether polyurethane, polyester polyurethane, polyethylene copolymer, polyamide, polyvinyl alcohol, polypropylene glycol, polytetramethylene oxide, polyvinyl pyrrolidone, polyacrylamide, poly(hydroxyethyl acrylate), poly(hydroxyethyl methacrylate), poly(glycolic acid), poly(DL-lactic-co-glycolic acid), polyhydroxyalkanoate, poly(4-hydroxybutirate), sulfonated polymers, polygluconic acid, poly(acrylic acid), polyphosphazenes, polysaccharides, proteins, collagen, elastin, alginate, fibrin, fibronectin, laminin, hyaluronic acid, or another biologically-occurring polymer, polypeptide sequences cleavable by proteases including matrix metalloproteases, or copolymers formed from monomers of one or more of these. For example, the hydrogel substrate may be comprised of polyamide monomers. In another example, the hydrogel substrate may comprise a combination of polyvinyl pyrrolidone and polyacrylamide. The hydrogel may also be polymerized from highly polydisperse precursors. For example, the hydrogel may be polymerized from polyethyle glycol vinyl sulfone precursors of varying sizes. The hydrogel may be a non-swelling hydrogel. But in some cases, the hydrogel is a swelling hydrogel.

In some instances, the polymer may comprise a chemically modified monomer, said monomer being substituted with, but not limited to, a sulfhydryl, vinyl sulfone, carboxylic acid, alcohol, vinyl, ester, thiol, or bromine group. For example, the polymer may comprise a hyaluronic acid substituted with a thiol group. In another example, the polymer may comprise a polyethylene glycol substituted with an ester group. In another example, the hydrogel polymer is composed of at least two different polyethylene glycol monomers. For example, the hydrogel may comprise polyethylene glycol sulfhydryl (PEG-SH) monomer and polyethylene glycol vinyl sulfone (PEG-VS) monomer.

The hydrogel substrate may be produced by reacting one or more polymers. For example, the hydrogel substrate is produced by reacting polyacrylamide monomers. In another example, the hydrogel substrate is produced by reacting hyaluronic acid and poly(vinyl acetate). In another example, the elastic hydrogel substrate is produced by reacting at least two polyethylene glycol compounds. For example, the sulfhydryl groups on 4-armed PEG (PEG-SH) are reacted with the vinyl sulfone groups on 8-armed PEG (PEG-VS) to produce the hydrogel substrate.

The water composition in the hydrogel substrate may vary. Hydrogels can contain over 70% water. Hydrogels can contain over 71% water. Hydrogels can contain over 72% water. Hydrogels can contain over 73% water. Hydrogels can contain over 74% water. Hydrogels can contain over 75% water. Hydrogels can contain over 76% water. Hydrogels can contain over 77% water. Hydrogels can contain over 78% water. Hydrogels can contain over 79% water. Hydrogels can contain over 80% water. Hydrogels can contain over 81% water. Hydrogels can contain over 82% water. Hydrogels can contain over 83% water. Hydrogels can contain over 84% water. Hydrogels can contain over 85% water. Hydrogels can contain over 90% water. Hydrogels can contain over 95% water. Hydrogels can contain over 99% water.

The hydrogel substrate is sufficiently thick to mask the physical properties of a container, device or tool. The hydrogel substrate is at least 5 μm thick. The hydrogel substrate is at least 10 μm thick. The hydrogel substrate is at least 20 μm thick. The hydrogel substrate is at least 30 μm thick. The hydrogel substrate is at least 40 μm thick. The hydrogel substrate is at least 50 μm thick. The hydrogel substrate is at least 100 μm thick. The hydrogel substrate is at least 200 μm thick. The hydrogel substrate is at least 300 μm thick. The hydrogel substrate is at least 400 μm thick. The hydrogel substrate is at least 500 μm thick. The hydrogel substrate is at least 1000 μm thick. The hydrogel substrate is at least 2000 μm thick. The hydrogel substrate is at least 3000 μm thick. The hydrogel substrate is at least 4000 μm thick. The hydrogel substrate is at least 5000 μm thick.

In some instances, the elasticity of the cell culture substrate (e.g., hydrogel) may be tuned (e.g., the elasticity of the substrate may be increased or decreased). In some cases, the tunability can be performed by the use of bonds which are unstable and therefore break over time. For example, the elasticity of the substrate can be tuned by the use of covalent bonds, ionic bonds, van der Waal's bonds, or any combination thereof. In other instances, the tunability can be performed by the use of chemical moieties which form bonds over time. For example, the addition of a polypeptide may decrease the elasticity of the substrate (e.g., make it more rigid). The elasticity of the substrate may be tuned by the addition or removal of ions or molecules which stabilize or destabilize bonds in the substrate. For example, the addition or removal of a biomolecule, such as collagen to, may cause a change in elasticity. The elasticity of the substrate may also be varied by the addition, removal, activation, or inactivation of enzymes which digest or form bonds in the substrate. Heating or cooling the elastic substrate is also another method to alter the elasticity of the substrate. For example, heating the substrate may increase the elasticity of the substrate (e.g. increase its flexibility). The elasticity of the substrate may also be modified by exposure to light which lyses or aids formation of bonds in the substrate. Exposure of the substrate to a catalyst which accelerates the formation of breakage of bonds in the substrate may also alter the elasticity of the substrate. Changes in ionic strength or pH of the cell culture medium may also be used to tune the elasticity of the substrate. The elasticity of the substrate may also be tuned by altering the composition. For example, a cell culture substrate comprising a hydrogel may be tuned by changing the polymer concentration (e.g., 0.01% polymer, 2% polymer, 5% polymer, etc). The elasticity of the substrate may also be tuned by modifying the length of the monomer. For example, varying the length of the polyethylene glycol monomer may increase or decrease the elasticity of the cell culture substrate. The cell culture substrate may also be tuned by modifying the branching of the polymer. For example, using a 2-arm, 4-arm, or 8-arm polyethylene glycol vinyl sulfone.

The elasticity of the cell culture substrate may be tuned before, during, or after the addition of a cell, biomolecule, or chemical. For example, the cell culture substrate may be tuned by the breakage of hydrogen bonds before the addition of a biomolecule. In another example, the cell culture substrate may be tuned by heating after the addition of the chemical. In another example, the cell culture substrate by be tuned by changing the pH of the cell culture media throughout the cell culturing process.

The elasticity of the hydrogel substrate may be tuned to sufficiently culture the cell on the hydrogel substrate. The elasticity of the hydrogel substrate may range from 0.01 kPa to 2000 kPa. For example, the elasticity range for culturing an endometrium cell may be 0.01 kPa to 10 kPa. In another example, elasticity range for culturing a smooth muscle cell may be 2 kPa to 100 kPa. In another example, the elasticity range for culturing an articular cartilage cell may be 500 kPa to 1500 kPa.

The elasticity of the hydrogel substrate may be varied before, during, or after the seeding of the cells. In some cases, the elasticity is varied over time while the cells are in culture. For example, the elasticity may be varied over the course of 1 minute, over the course of 10 minutes, over the course of 1 hour, over the course of 1 week, over the course of 1 month, over the course of 2 months, over the course of 3 months, over the course of 4 months, over the course of 5 months, over the course of 6 months, etc. The elasticity may be increased by 5% or more, by 10% or more, by 15% or more, or by 20% or more, in some instances by 30% or more, by 40% or more, or by 50%, in some instances by more than 50%, e.g. by 60% or more, by 70% or more, by 80% or more; by 90% or more, by 100% or more, sometimes by more than 100%, e.g. by 200% or more. The elasticity may be decreased by 5% or more, by 10% or more, by 15% or more, or by 20% or more, in some instances by 30% or more, by 40% or more, or by 50%, in some instances by more than 50%, e.g. by 60% or more, by 70% or more, by 80% or more, by 90% or more, by 100% or more, sometimes by more than 100%, e.g. by 200% or more.

Elastic substrates (e.g., hydrogels) may also be patterned with regions of different elasticities. For example, the cell culture substrate may have an elasticity of 0.1 kPa in one region and an elasticity of 4 kPa in another region. and may be formed in a variety of shapes, e.g. cilia, folds, crypts, striations, processes, dimples, partial spherical shells, and bumps, which optionally mimic a native environment of the cell. The substrate may also be subjected to twisting, compressing, stretching, or otherwise deforming the elastic substrate during culture. Medium may be added to the substrate prior to, or at the time when cells are brought into contact. Cells may be deposited on a substrate surface, sandwiched between two or more substrates with similar or different elasticities, or encapsulated in the substrate for both in vitro and in vivo purposes.

### Additions to the Hydrogel Substrate

The hydrogel substrate may further comprise a biomolecule, chemical, or any combination thereof linked to its surface. The biomolecule may be, but is not limited to, a protein, a polypeptide, a peptide, a nucleic acid molecule, a nucleotide, an oligonucleotide, a polynucleotide, a saccharaide, a polysaccharide, a cytokine, a growth factor, a morphogens, an antibody, a peptibody, or any fragment thereof. The cytokine may be without limitation erythropoietin, granulocyte-macrophage colony stimulating factor, granulocyte colony stimulating factor, macrophage colony stimulating factor, thrombopoietin, stem cell factor, interleukin-1, interleukin-2, interleukin-3, interleukin-6, interleukin-7, interleukin-15, Flt3L, leukemia inhibitory factor, insulin-like growth factor, insulin, or any fragment thereof. The morphogens may be, but are not limited to, transforming growth factor beta (TGF-beta), Hedgehog/Sonic Hedgehog, Wingless/Wnt, epidermal growth factor (EGF), and fibroblast growth factor (FGF), or any fragment thereof. Growth factors include, but are not limited to, fibroblast growth factor, epidermal growth factor, insulin-like growth factor 1, platelet-derived growth factor, nerve growth factor, transforming growth factor beta, or any fragment thereof. In some embodiments, the protein is selected from the native cellular environment or niche, including, but not limited, fibronectin, laminin, collagen, vitronectin, entactin, bone morphogenetic protein, bone morphogenetic protein ligand, E-cadherin, extracellular matrix proteins, or any fragment thereof.

### Kits

In some instances, a cell culture kit comprising a container and the cell culture substrate disclosed herein is provided. In some cases, the container is a cell culture dish, multi-well plate, flask, centrifuge tube, microcentrifuge tube, beaker, test tube, or cryovial. In other instances, a kit comprising a tool and the cell culture substrate may be provided. In some cases, the tool may be a pipet (e.g., a holding pipet or an injection pipet).

In some cases, the kits described herein may further comprise a chemical linked to the cell culture substrate surface. The chemical may be, but is not limited to, a maleimide, aspartate, glutamate, carboxylic acid, and dicarboxylic acid.

In some instances, the kits described herein may further comprise a cell attached to or encapsulated in the cell culture substrate. In some instances, the cell may be a stem cell, primary cell, transdifferentiated cell, dedifferentiated cell, reprogrammed cell, multipotent cell, or pluripotent cell. For example, a cell culture kit may comprise a stem cell attached to hydrogel substrate contained in a cell culture dish. In another example, a kit may comprise an oocyte inside a holding pipet coated with an elastic substrate.

The cell culture kit may further comprise a biomolecule attached to the surface of the cell culture substrate. The biomolecule may be a protein, a polypeptide, a peptide, a nucleic acid molecule, a nucleotide, an oligonucleotide, a polynucleotide, a saccharide, a polysaccharide, a cytokine, a growth factor, a morphogens, an antibody, a peptibody, or any fragment thereof. For example, a cell culture kit may comprise collagen linked to the cell culture substrate contained within a multiwell plate.

Cells cultured in the containers of the invention find various uses, e.g. may be implanted or transplanted into a recipient, used in the formation of engineered tissue, transferred for further culture, and the like.

### Monitoring

Methods of culturing cells on a cell culture substrate may further comprise monitoring the cells, use of the cells in assisted reproductive technology, or screening assays. The methods to monitor the cells include, but are not limited to, a digital camera, a video camera, a temperature sensor, a pH sensor, or a sensor of concentrations of metabolites or other medium constituents. The expansion of individual cells can be tracked in the culture methods of the invention, so as to identify the parameters of individual cells, using the Baxter algorithm data analysis method described herein. For example, the trajectories of individual cells are tracked, and the unique identity of individual cells is determined through the tracking, so that parameters of individual cells can be extracted from the data. The software provides the data processing necessary to analyze cell behavior, from preprocessing images to automatically tracking cells and correcting the obtained cell trajectories.

Prior to analysis images are stabilized to correct for camera shake. Microwell outlines are identified, and the image background removed to prevent stationary debris and microwell edges from being perceived as cells. The background image is computed by taking the median pixel intensity in the time dimension for every pixel in the image. To find moving cells from brightfield images, Gaussian smoothing is applied to the absolute value of the background subtracted images. Then a modified, seeded watershed algorithm is used on the negative of this image. Seeds are put on local minima below a certain threshold, set by looking at the intensities of minima inside cells and in the background of representative images. The probability that a region contains 0, 1 or more than 1 cell is computed, using multinomial logistic regression, using the parameters: 1) (area)/(average area in all regions of the sequence); 2) (area of convex hull)/(area); 3) (perimeter)/(radius of circle with the same area); 4) (distance from microwell center)/(microwell radius); 5) average absolute gradient component parallel to boundary; 6) average absolute gradient component perpendicular to boundary; and 7) (mean intensity)−(mean intensity in all regions of the sequence). Cell trajectories are then added one by one. The construction and modification of these trajectories was done to try and maximize the utility function.

$$S = {\sum }_{c = 1}^{cellcount} [ log p ( {D}_{c} = 1 ) + {\sum }_{t = {t}_{first}}^{{t}_{last}} log p ( {T}_{c}^{t} = 1 ) ] + {\sum }_{b = 1}^{blobcount} log p ( {N}_{b} = {n}_{b} )$$

Here p(N_{b}=n_{b}) is the probability that blob b contains n_{b} cells, estimated using multinomial logistic regression as described above. If the hypothesized cell c divides, p(Dc=1) is the estimated probability that the division is correct. Otherwise p(Dc=1) is set to 1, so that nothing is subtracted from S. The division probability is estimated from the distance traveled by the cell during the 30 minutes preceding the division and the distance between the cell and the resulting daughter cells. p(T_{c}^{t}=1) is the probability that the movement cell c makes in frame t occurred, estimated from the distance between the cell positions in the two frames.

To model the proliferation and death of cells in a culture condition we assume that the cells have a proliferation rate rₚ and a death rate r_{d}, and that these rates are not dependent on time. If the cell number as a function of time is c(t) we find that its derivative is

$$\frac{d c}{d t} = ( {r}_{p} - {r}_{d} ) c .$$

With a cell number of c₀ at t=0, we find that c(t)=c₀ exp((rₚ−r_{d})t). Given that we have n cell observations in total, nₚ observations of cell division and n_{d} observations of cell death, we can estimate rₚ and r_{d} as

$${r}_{p} = a \frac{{n}_{p}}{n} and {r}_{d} = a \frac{{n}_{d}}{n} ,$$

where a is the number of frames per time unit, and conventional analysis was used to determine the statistical significance of the modeled proliferation and death rates

Following expansion the cells may be removed from the surface of the substrate by digestion with enzymes, chelators, etc., as known in the art using time, temperature, concentration and selection of reagents that will achieve a partial digestion that leaves aggregates of cells. One of skill in the art can readily perform a simple titration to determine suitable conditions, e.g. using EDTA, elastase; dispase; collagenase; trypsin; blendzyme; and the like.

These cells may find various applications for a wide variety of purposes. The cell populations may be used for screening various additives for their effect on growth and the mature differentiation of the cells. In this manner, compounds which are complementary, agonistic, antagonistic or inactive may be screened, determining the effect of the compound in relationship with one or more of the different cytokines.

The populations may be employed as grafts for transplantation. For example, muscle cells find use in the regeneration or treatment of muscle, hematopoietic cells are used to treat malignancies, bone marrow failure states and congenital metabolic, immunologic and hematologic disorders; and the like.

For therapeutic methods the cells may be used for tissue reconstitution or regeneration in a human patient or other subject in need of such treatment. The cells are administered in a manner that permits them to graft or migrate to the intended tissue site and reconstitute or regenerate the functionally deficient area.

The differentiating cells may be administered in any physiologically acceptable excipient, where the cells may find an appropriate site for regeneration and differentiation. The cells may be introduced by injection, catheter, or the like. The cells may be frozen at liquid nitrogen temperatures and stored for long periods of time, being capable of use on thawing. If frozen, the cells may usually be stored in any suitable medium, for example 10% DMSO, 20% FCS, 70% DMEM medium. Once thawed, the cells may be expanded by use of growth factors and/or feeder cells associated with progenitor cell proliferation and differentiation.

The cells of this invention are generally a defined highly enriched FACS sorted population. The cells can be supplied in the form of a pharmaceutical composition, comprising an isotonic excipient prepared under sufficiently sterile conditions for human administration. Choice of the cellular excipient and any accompanying elements of the composition will be adapted in accordance with the route and device used for administration. The composition may also comprise or be accompanied with one or more other ingredients that facilitate the engraftment or functional mobilization of the cells. Suitable ingredients include matrix proteins that support or promote adhesion of the cells.

The subject methods are useful for both prophylactic and therapeutic purposes. Thus, as used herein, the term “treating” is used to refer to both prevention of disease, and treatment of a pre-existing condition. The treatment of ongoing disease, to stabilize or improve the clinical symptoms of the patient, is a particularly important benefit provided by the present invention. Such treatment is desirably performed prior to loss of function in the affected tissues; consequently, the prophylactic therapeutic benefits provided by the invention are also important. Evidence of therapeutic effect may be any diminution in the severity of disease. The therapeutic effect can be measured in terms of clinical outcome or can be determined by immunological or biochemical tests.

The dosage of the therapeutic formulation will vary widely, depending upon the nature of the condition, the frequency of administration, the manner of administration, the clearance of the agent from the host, and the like. The initial dose can be larger, followed by smaller maintenance doses. The dose can be administered as infrequently as weekly or biweekly, or more often fractionated into smaller doses and administered daily, semi-weekly, or otherwise as needed to maintain an effective dosage level.

### Assisted Reproductive Treatment Methods

Methods for ex vivo manipulation of reproductive cells are included in the invention. Such reproductive cells include, but are not limited to, germ cells, fertilized oocytes, gametes, gametocytes, embryos, zygotes and the like. Concepti include sperm-penetrated oocyte, pre-zygote, fertilized oocyte, pre-embryo, embryo, cleavage stage embryo, pre-morula, morula, early blastocyst, blastocyst, hatched blastocyst, or concepti derived therefrom. The term “cells” refers both to single cells, and to tissues or organisms such as embryos, embryoid bodies, ovarian follicles, pieces of ovarian tissue, etc. The substrate compositions, containers and devices are as described herein.

Methods include seeding gametes, concepti, or other reproductive cells on an elastic substrate, where the elasticity of the substrate mimics the elasticity of the in vivo microenvironment of the gamete, concepti, or other reproductive cells. For example, a gamete is seeded on a cell culture substrate, which mimics the elasticity of the gamete's in vivo microenvironment. Fertilization may be performed prior to, or following the seeding on an elastic substrate by the addition of sperm cells to the medium, or by the microinjection of sperm. The medium may include factors that activate sperm cells, as known in the art and/or factors that activate oocytes or follicles by factors, chemical means, etc. as known in the art. In some embodiments, a procedure may be performed to strip the cumulus oophorous from one or more of the oocytes in cumulus-oocyte complexes prior to fertilization. Assisted zona hatching may be performed on the one or more oocytes, cumulus-oocyte complexes, or concepti.

The cells of interest may be co-cultured with suitable cells including those derived from follicular cells, endometrial cells, uterine cells, endocrine cells, stem cells, or a cell line selected for its ability to secrete molecules which influence gametes or concepti, or other reproductive cells.

The microenvironment elasticity that is mimicked, or approximated, for these purposes may include perivitelline space, zona pellucida, corona radiata, cumulus oophorous, ovarian follicle, fimbriae of a fallopian tube, an infundibulum, the ampulla or isthmus of a fallopian tube, a uterus, an endometrium, or an endometrial crypt, seminal vesicles, testes, epididymus, vas deferens, seminal vesicle, ejaculatory duct, prostate gland, urethra, cowper's gland, or mucus or other fluids found in any of these structures. The range of elasticity may be at least about 0.001 kPa, at least about 0.1 kPa, at least about 1 kPa, and not more than about 1000 kPa, not more than about 100 kPa, not more than about 50 kPa.

The gametes or resulting concepti (or other reproductive cell) may be maintained on the substrate in a suitable culture medium for a period of time, e.g. the period of time sufficient to mature the embryo to a stage appropriate for implantation, usually about 1, 2, 3, 4, 5, 6, 7 days, for example to the blastocyst stage. The reproductive cells may be subjected to various procedures of interest, such as, but not limited to, cytoplasmic transfer between gametes, embryo splitting, exposure to drugs which may enhance fertilization or pregnancy, etc. At such stage the embryo may be implanted in a suitable recipient, utilized for testing and analysis as known in the art, cultured to generate in vitro embryonic stem cell lines, frozen, etc. Analysis may include preimplantation genetic diagnosis (PGD). The reproductive cells may be frozen in a conventional container or in a container comprising an elastic substrate of the invention, and may be thawed in a conventional container or a container comprising an elastic substrate of the invention.

For implantation in a recipient, the cells may be separated from the substrate prior to implantation, or may be implanted with the substrate or a portion thereof, or may be initially contacted with the recipient in the presence of a substrate that is then washed away, for example by an attached transfer device that allows fluid to be forced through the substrate to aid detachment of the one or more gametes or concepti from the substrate. Implantation may include gamete intrafallopian transfer (GIFT), or zygote intrafallopian transfer (ZIFT).

The cells may be monitored automatically or semi-automatically, which may include the analysis methods described herein, where the data may be used to inform decisions regarding fertilization and implantation procedures.

Another method of assisted reproduction comprises culturing a reproductive cell on cell culture substrate, where the elastic substrate may be varied after the addition of the reproductive cell, a biomolecule, or a chemical. The biomolecule may be, but is not limited to, a protein, a polypeptide, a peptide, a nucleic acid molecule, a nucleotide, an oligonucleotide, a polynucleotide, a saccharide, a polysaccharide, a cytokine, a growth factor, a morphogens, an antibody, a peptibody, or any fragment thereof. For example, a gamete is cultured on a cell culture substrate containing a carbohydrate linked to its surface.

The reproduction methods and compositions provided herein may be useful for improving pregnancy rates, reducing miscarriage rates, and reducing the incidence of birth defects. In some cases, the pregnancy rates are improved for a specific population, e.g., women over, or under, the age of 20, 25, 30, 35, 40, 41, 42, 43, 44, 45, 50, or 55. In some cases, the pregnancy rates are improved by greater than 2-fold, 3-fold, 4-fold, 5-fold, 6-fold, 7-fold, 10-fold, or more.

### Screening Methods

In some instances, the cells cultured on the elastic substrate may be used for screening various additives for their effect on maintenance, expansion, growth and differentiation of the cells. For example, compounds which are complementary, agonistic, antagonistic or inactive may be screened, determining the effect of the compound in relationship with one or more of the different cytokines. Cultured cells may be contacted with a variety of biologically active entities, including, but not limited to, growth factors, cytokines, drugs, proteins, polymers, cells, polysaccharides, nanoparticles, antibodies, ion channel blockers, receptor ligands, shRNA, siRNA, miRNA, nucleic acids, dyes, lipids, etc.

Candidate agents may be screened for their effect on cells in the cultures of the invention. For example, candidate agents may be screened for their effect on stem cell differentiation. For example, candidate agents may be screened for their effect on cancer cells or infected cells proliferation. The effect of an agent is determined by adding the agents to the cell cultures described herein, usually in conjunction with a control cell culture lacking the agent. The growth of the cell may be analyzed visually, or using the methods of analysis described herein. The change in growth, differentiation, gene expression, proteome, phenotype with respect to markers, transport of agents, etc. in response to the agent is measured and evaluated by comparison to control stem cell culture. Agents of interest for analysis include any biologically active molecule with the capability of modulating, directly or indirectly, the growth rate of the cells, for example genetic agents, monoclonal antibodies, protein factors, small molecule therapeutics, chemotherapeutics, radiation, anti-sense RNA, RNAi, and the like.

In another example, the cells are infected with a pathogen (bacterial or viral). Candidate agents are screened for anti-bacterial or anti-viral activity. Anti-bacterial or anti-viral activity of an agent can be assessed by monitoring growth, ultrastructure and viability of the explants.

In a certain application of the culture system, the culture system is used to assess whether certain agents cause cell toxicity. In these applications, the cell culture is exposed to the candidate agent or the vehicle and its growth and viability is assessed. In these applications, analysis of the ultrastructure of the cells is also useful.

The agents are conveniently added in solution, or readily soluble form, to the medium of cells in culture. The agents may be added in a flow-through system, as a stream, intermittent or continuous, or alternatively, adding a bolus of the compound, singly or incrementally, to an otherwise static solution. In a flow-through system, two fluids are used, where one is a physiologically neutral solution, and the other is the same solution with the test compound added. The first fluid is passed over the cells, followed by the second. In a single solution method, a bolus of the test compound is added to the volume of medium surrounding the cells. The overall concentrations of the components of the culture medium should not change significantly with the addition of the bolus, or between the two solutions in a flow-through method. Alternatively, the agents can be injected into the lumen of the muscle stem cysts and their effect compared to injection of controls.

Preferred agent formulations do not include additional components, such as preservatives, that may have a significant effect on the overall formulation. Thus preferred formulations consist essentially of a biologically active compound and a physiologically acceptable carrier, e.g. water, ethanol, DMSO, etc. However, if a compound is liquid without a solvent, the formulation may consist essentially of the compound itself.

A plurality of assays may be run in parallel with different agent concentrations to obtain a differential response to the various concentrations. As known in the art, determining the effective concentration of an agent typically uses a range of concentrations resulting from 1:10, or other log scale, dilutions. The concentrations may be further refined with a second series of dilutions, if necessary. Typically, one of these concentrations serves as a negative control, i.e. at zero concentration or below the level of detection of the agent or at or below the concentration of agent that does not give a detectable change in the growth rate.

Candidate agents may be biologically active agents that encompass numerous chemical classes, organic molecules, which may include organometallic molecules, inorganic molecules, genetic sequences, etc. Another aspect of the invention is to evaluate candidate drugs, select therapeutic antibodies and protein-based therapeutics, for their effect on cells. Candidate agents may comprise functional groups necessary for structural interaction with proteins, particularly hydrogen bonding, and typically include at least an amine, carbonyl, hydroxyl or carboxyl group, frequently at least two of the functional chemical groups. The candidate agents may comprise cyclical carbon or heterocyclic structures and/or aromatic or polyaromatic structures substituted with one or more of the above functional groups. Candidate agents are also found among biomolecules, including peptides, polynucleotides, saccharides, fatty acids, steroids, purines, pyrimidines, derivatives, structural analogs or combinations thereof. Included are pharmacologically active drugs, genetically active molecules, etc. Compounds of interest include chemotherapeutic agents, anti-inflammatory agents, hormones or hormone antagonists, ion channel modifiers, and neuroactive agents.

Compounds, including candidate agents, are obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Candidate agent can also be polynucleotides and analogs thereof, which are tested in the screening assays of the invention by addition of the genetic agent to the muscle stem cell culture. The introduction of the genetic agent can result in an alteration of the total genetic composition of the cell. Genetic agents such as DNA can result in an experimentally introduced change in the genome of a cell, generally through the integration of the sequence into a chromosome. Genetic changes can also be transient, where the exogenous sequence is not integrated but is maintained as episomal agents. Genetic agents, such as antisense oligonucleotides, can also affect the expression of proteins without changing the cell's genotype, by interfering with the transcription or translation of mRNA. Genetic agents, such as short interfering RNA (siRNA) or short hairpin (shRNA), can effect expression of proteins without changing the cell's genotype by mediated the degradation of the mRNA it binds to. The effect of a genetic agent is to increase or decrease expression of one or more gene products in the cell.

Introduction of an expression vector encoding a polypeptide can be used to express the encoded product in cells lacking the sequence, or to over-express the product. Various promoters can be used that are constitutive or subject to external regulation, where in the latter situation, one can turn on or off the transcription of a gene. These coding sequences may include full-length cDNA or genomic clones, fragments derived therefrom, or chimeras that combine a naturally occurring sequence with functional or-structural domains of other coding sequences. Alternatively, the introduced sequence may encode an anti-sense sequence; be an anti-sense oligonucleotide; siRNA or a shRNA, encode a dominant negative mutation, or dominant or constitutively active mutations of native sequences; altered regulatory sequences, etc. Instead of being expressed from a vector transfected or transduced into the muscle stem epithelial cells, the oligonucleotides, siRNA or shRNA can be directly transfected or transduced into the muscle stem cells.

In addition to sequences derived from the host cell species, other sequences of interest include, for example, genetic sequences of pathogens, for example coding regions of viral, bacterial and protozoan genes, particularly where the genes affect the function of human or other host cells. Sequences from other species may also be introduced, where there may or may not be a corresponding homologous sequence.

A large number of public resources are available as a source of genetic sequences, e.g. for human, other mammalian, and human pathogen sequences. A substantial portion of the human genome is sequenced, and can be accessed through public databases such as Genbank. Resources include the uni-gene set, as well as genomic sequences. For example, see Dunham et al. (1999) Nature 402, 489-495; or Deloukas et al. (1998) Science 282, 744-746.

cDNA clones corresponding to many human gene sequences are available from the IMAGE consortium. The international IMAGE Consortium laboratories develop and array cDNA clones for worldwide use. The clones are commercially available, for example from Genome Systems, Inc., St. Louis, Mo. Methods for cloning sequences by PCR based on DNA sequence information are also known in the art.

Methods that are well known to those skilled in the art can be used to construct expression vectors containing coding sequences and appropriate transcriptional and translational control signals for increased expression of an exogenous gene introduced into a cell. These methods include, for example, in vitro recombinant DNA techniques, synthetic techniques, and in vivo genetic recombination. Alternatively, RNA capable of encoding gene product sequences may be chemically synthesized using, for example, synthesizers. See, for example, the techniques described in “Oligonucleotide Synthesis”, 1984, Gait, M. J. ed., IRL Press, Oxford.

A variety of host-expression vector systems may be utilized to express a genetic coding sequence. Expression constructs may contain promoters derived from the genome of mammalian cells, e.g., metallothionein promoter, elongation factor promoter, actin promoter, etc., from mammalian viruses, e.g., the adenovirus late promoter; the vaccinia virus 7.5K promoter, SV40 late promoter, cytomegalovirus, etc.

In mammalian host cells, a number of viral-based expression systems may be utilized, e.g. retrovirus, lentivirus, adenovirus, herpesvirus, and the like. In cases where an adenovirus is used as an expression vector, the coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the gene product in infected hosts (see Logan & Shenk, 1984, Proc. Natl. Acad. Sci. USA 81:3655-3659). Specific initiation signals may also be required for efficient translation of inserted gene product coding sequences. These signals include the ATG initiation codon and adjacent sequences. Standard systems for generating adenoviral vectors for expression on inserted sequences are available from commercial sources, for example the Adeno-X™ expression system from Clontech (Clontechniques, January 2000, p. 10-12).

In cases where an entire gene, including its own initiation codon and adjacent sequences, is inserted into the appropriate expression vector, no additional translational control signals may be needed. However, in cases where only a portion of the gene coding sequence is inserted, exogenous translational control signals, including, perhaps, the ATG initiation codon, must be provided. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., 1987, Methods in Enzymol. 153:516-544).

In a preferred embodiment, methods are used that achieve a high efficiency of transfection, and therefore circumvent the need for using selectable markers. These may include adenovirus infection (see, for example Wrighton, 1996, J. Exp. Med. 183: 1013; Soares, J. Immunol., 1998, 161: 4572; Spiecker, 2000, J. Immunol 164: 3316; and Weber, 1999, Blood 93: 3685); and lentivirus infection (for example, International Patent Application WO000600; or WO9851810). Adenovirus-mediated gene transduction of endothelial cells has been reported with 100% efficiency. Retroviral vectors also can have a high efficiency of infection with endothelial cells, provides virtually 100% report a 40-77% efficiency. Other vectors of interest include lentiviral vectors, for examples, see Barry et al. (2000) Hum Gene Ther 11(2):323-32; and Wang et al. (2000) Gene Ther 7(3):196-200.

For the purpose of analysis of the effect of gene over-expression introduction of the test gene into a majority of cells (>50%) in a culture is sufficient. This can be achieved using viral vectors, including retroviral vectors (e.g. derived from MoMLV, MSCV, SFFV, MPSV, SNV etc), lentiviral vectors (e.g. derived from HIV-1, HIV-2, SIV, BIV, FIV etc.), adeno-associated virus (AAV) vectors, adenoviral vectors (e.g. derived from Ad5 virus), SV40-based vectors, Herpes Simplex Virus (HSV)-based vectors etc. A preferred vector construct will coordinately express a test gene and a marker gene such that expression of the marker gene can be used as an indicator for the expression of the test gene, as well as for analysis of gene transfer efficiency. This can be achieved by linking the test and a marker gene with an internal ribosomal entry site (IRES) sequence and expressing both genes from a single bi-cistronic mRNA. IRES sequence could be from a virus (e.g. EMCV, FMDV etc) or a cellular gene (e.g. eIF4G, BiP, Kv1.4 etc). The examples of marker genes include drug resistance genes (neo, dhfr, hprt, gpt, bleo, puro etc) enzymes (β-galactosidase, alkaline phosphatase etc) fluorescent genes (e.g. GFP, RFP, BFP, YFP) or surface markers (e.g. CD24, NGFr, Lyt-2 etc). A preferred marker gene is biologically inactive and can be detected by standard immunological methods. Alternatively, an “epitope tag” could be added to the test gene for detection of protein expression. Examples of such “epitope tags” are c-myc and FLAG (Stratagene). A preferred viral vector will have minimal or no biological effect on the biomap apart from the genetic agent being tested. An example of such viral vectors are retroviral vectors derived from the MoMLV or related retroviruses, as listed above. By gating on the population of genetically modified cells, the unmodified cells in the culture can be excluded from analysis, or can be compared directly with the genetically modified cells in the same assay combination. For example, see Bowman et al. (1998) J. Biol. Chem. 273:28040-28048.

### Screening Candidate Populations for Presence of Stem Cells

In another embodiment of the invention the cultures of the invention are used to screen candidate cell populations for the presence of stem cells (or other cells) or the potential to develop into stem cells (or other type of cells). Candidate cell populations are screened by adding the cells to the cultures described above, usually in conjunction with a control culture lacking the candidate cell or seeded with a known stem cell. The presence of muscle stem stem cells among candidate cells can be assayed by increase in the number of transplantable cells, analysis of multi-lineage differentiation, and analysis of long term proliferation.

Candidate cells can be detectably marked, for example via expression of a marker such as GFP or β-galactosidase. Candidate cells marked via expression of GFP are derived by standard techniques. GFP transduced candidate cells can be generated by techniques well known in the art, for example using a viral vector expressing GFP. Labeled candidate cells may be co-cultured with non-labeled cells. Cells may be introduced in a limiting dilution, or as a population, e.g. 1, 5, 10, 100, 500, 1000 or more cells per culture. The cells may be cultured for about 1, 2, 3, 4, 5, 6, 7, 8, 9, 10, 11, 12, or more weeks prior to evaluation for expansion or differentiation.

The assessment of the candidate cells may be performed by visual observation, computer assisted observation, determination of expression of various differentiation markers, and the like. Immunofluorescence can be performed using antibodies against stem cell markers. Dual color immunofluorescence may be performed with the intrinsic GFP signal to confirm co-localization of differentiation markers with candidate cells.

Methods of in vivo analysis include various methods where cells are transferred to an in vivo environment. In some embodiments, stem cells are cultured using the methods described above, extracted from the hydrogel, and implanted in an experimental animal, e.g. syngeneic or immunodeficient mice, then allowed to grow for a suitable period of time, e.g. at least about 1 week, at least about 2 weeks, at least about 3-4 weeks, at least about 1, 2, 3, 4 or more months, etc. This assay can be modified to utilize various marker systems, e.g. luciferase expressing cells that permit periodic non-invasive imaging after luciferin injection. Growth and serial transplantability is compared between cultured cells, freshly isolated cells, control cultures, etc.

### Disease Conditions

In certain embodiments, the methods, compositions, or cells disclosed herein may be used to treat diseases or conditions. In some embodiments, diseases or conditions include, but are not limited to, muscle disorders, neurodegenerative disorders, hematologic disorders, genetic disorders, cardiovascular disease, inflammatory diseases, diabetes, skin conditions, and reproductive conditions.

Diseases of interest for treatment with muscle stem cells, particularly allogeneic cells and/or genetically modified autologous cells, include heritable and acquired muscle disorders. A number of muscle conditions in which there is muscle wasting such as atrophy and sarcopenia, are of interest, e.g. conditions associated with increased age, immobility, drug treatment, cancer, and the like.

In some embodiments, the inherited muscle disorders include, without limitation, muscular dystrophies. For example, Duchenne dystrophy is an X-linked recessive disorder characterized by progressive proximal muscle weakness with destruction and regeneration of muscle fibers and replacement by connective tissue. Duchenne dystrophy is caused by a mutation at the Xp21 locus, which results in the absence of dystrophin, a protein found inside the muscle cell membrane. It affects 1 in 3000 live male births. Symptoms typically start in boys aged 3 to 7 yr. Progression is steady, and limb flexion contractures and scoliosis develop. Firm pseudohypertrophy (fatty and fibrous replacement of certain enlarged muscle groups, notably the calves) develops. Becker muscular dystrophy is a less severe variant, also due to a mutation at the Xp21 locus. Dystrophin is reduced in quantity or in molecular weight. Patients usually remain ambulatory, and most survive into their 30s and 40s.

In some embodiments, muscle disorders include myopathies. In some embodiments, myopathies include, but are not limited to,congenital and metabolic myopathies, including glycogen storage diseases and mitochondrial myopathies. Congenital myopathies are a heterogeneous group of disorders that cause hypotonia in infancy or weakness and delayed motor milestones later in childhood. An autosomal dominant form of nemaline myopathy is linked to chromosome 1 (tropomyosin gene), and a recessive form to chromosome 2. Other forms are caused by mutations in the gene for the ryanodine receptor (the calcium release channel of the sarcoplasmic reticulum) on chromosome 19q. Skeletal abnormalities and dysmorphic features are common. Diagnosis is made by histochemical and electron microscopic examination of a muscle sample to identify specific morphologic changes.

Mitochondrial myopathies range from mild, slowly progressive weakness of the extraocular muscles to severe, fatal infantile myopathies and multisystem encephalomyopathies. Some syndromes have been defined, with some overlap between them. Established syndromes affecting muscle include progressive external ophthalmoplegia, the Kearns-Sayre syndrome (with ophthalmoplegia, pigmentary retinopathy, cardiac conduction defects, cerebellar ataxia, and sensorineural deafness), the MELAS syndrome (mitochondrial encephalomyopathy, lactic acidosis, and stroke-like episodes), the MERFF syndrome (myoclonic epilepsy and ragged red fibers), limb-girdle distribution weakness, and infantile myopathy (benign or severe and fatal). Muscle biopsy specimens stained with modified Gomori's trichrome stain show ragged red fibers due to excessive accumulation of mitochondria. Biochemical defects in substrate transport and utilization, the Krebs cycle, oxidative phosphorylation, or the respiratory chain are detectable. Numerous mitochondrial DNA point mutations and deletions have been described, transmitted in a maternal, nonmendelian inheritance pattern. Mutations in nuclear-encoded mitochondrial enzymes occur.

Glycogen storage diseases of muscle are a group of rare autosomal recessive diseases characterized by abnormal accumulation of glycogen in skeletal muscle due to a specific biochemical defect in carbohydrate metabolism. These diseases can be mild or severe. In a severe form, acid maltase deficiency (Pompe's disease), in which 1,4-glucosidase is absent, is evident in the first year of life and is fatal by age 2. Glycogen accumulates in the heart, liver, muscles, and nerves. In a less severe form, this deficiency may produce proximal limb weakness and diaphragm involvement causing hypoventilation in adults. Myotonic discharges in paraspinal muscles are commonly seen on electromyogram, but myotonia does not occur clinically. Other enzyme deficiencies cause painful cramps after exercise, followed by myoglobinuria. The diagnosis is supported by an ischemic exercise test without an appropriate rise in serum lactate and is confirmed by demonstrating a specific enzyme abnormality.

Channelopathies are neuromuscular disorders with functional abnormalities due to disturbance of the membrane conduction system, resulting from mutations affecting ion channels. Myotonic disorders are characterized by abnormally slow relaxation after voluntary muscle contraction due to a muscle membrane abnormality.

Myotonic dystrophy (Steinert's disease) is an autosomal dominant multisystem disorder characterized by dystrophic muscle weakness and myotonia. The molecular defect is an expanded trinucleotide (CTG) repeat in the 3′ untranslated region of the myotonin-protein kinase gene on chromosome 19q. Symptoms can occur at any age, and the range of clinical severity is broad. Myotonia is prominent in the hand muscles, and ptosis is common even in mild cases. In severe cases, marked peripheral muscular weakness occurs, often with cataracts, premature balding, hatchet facies, cardiac arrhythmias, testicular atrophy, and endocrine abnormalities. Mental retardation is common. Severely affected persons die by their early 50s.

Myotonia congenita (Thomsen's disease) is a rare autosomal dominant myotonia that usually begins in infancy. In several families, the disorder has been linked to a region on chromosome 7 containing a skeletal muscle chloride channel gene. Painless muscle stiffness is most troublesome in the hands, legs, and eyelids and improves with exercise. Weakness is usually minimal. Muscles may become hypertrophied. Diagnosis is usually established by the characteristic physical appearance, by inability to release the handgrip rapidly, and by sustained muscle contraction after direct muscle percussion.

Familial periodic paralysis is a group of rare autosomal dominant disorders characterized by episodes of flaccid paralysis with loss of deep tendon reflexes and failure of muscle to respond to electrical stimulation. The hypokalemic form is due to genetic mutation in the dihydropyridine receptor-associated calcium channel gene on chromosome 1q. The hyperkalemic form is due to mutations in the gene on chromosome 17q that encodes a subunit of the skeletal muscle sodium channel (SCN4A).

Genetic disorders include, but are not limited to, Alzheimer's disease, amyotrophic lateral sclerosis, Friedreich's ataxia, Huntington's disease, Lewy body disease, Parkinson's disease, and spinal muscular atrophy.

Other diseases of interest include, but are not limited to: cardiovascular diseases such as cardiomyopathy, coronary artery disease, congenital heart disease, pericardial disease, and vascular disease; inflammatory disease; arthritis; dermatitis; Diabetes Type I and II; eye disease (e.g., macular degeneration); auditory disease, (e.g., deafness); cognitive impairment; mental illnesses such as schizophrenia, depression, bipolar disorder, or dementia; neurodegenerative diseases such as Alzheimer's Disease, Parkinson's Disease, multiple sclerosis; osteoporosis; liver disease (e.g., hepatitis, liver cancer); kidney disease (e.g., nephritis, kidney cancer); autoimmune disease; and proliferative disorders (e.g., a cancer).

Often, a subject provides cells for the use in the methods and compositions disclosed herein. The subject may be free of a disease condition. In other cases, the subject is suffering from, or at high risk of suffering from, a health condition or even an acute health condition e.g., stroke, spinal cord injury, burn, or a wound. In certain cases, a subject provides cells for his or her future use (e.g., an autologous therapy), or for the use of another subject who may need treatment or therapy (e.g., an allogeneic therapy).

In certain embodiments, the cell populations described herein may be employed as grafts for transplantation. For example, muscle cells find use in the regeneration or treatment of muscle, hematopoietic cells are used to treat malignancies, bone marrow failure states and congenital metabolic, immunologic and hematologic disorders; and the like. For therapeutic methods the cells may be used for tissue reconstitution or regeneration in a human patient or other subject in need of such treatment. The cells are administered in a manner that permits them to graft or migrate to the intended tissue site and reconstitute or regenerate the functionally deficient area.

Cells cultured on the cell culture substrate may be used for tissue reconstitution or regeneration. The cells are administered in a manner that permits them to graft or migrate to the intended tissue site and reconstitute or regenerate the functionally deficient area. Cells cultured on the cell substrate may also be injected, implanted, transplanted into or grafted onto an individual.

The differentiating cells may be administered in any physiologically acceptable excipient. The cells may be introduced by injection, catheter, or the like. For example, a muscle stem cell may be injected into the calf muscle of an individual and regenerate new muscle.

The cells can be supplied in the form of a pharmaceutical composition, comprising an isotonic excipient prepared under sufficiently sterile conditions for human administration. Choice of the cellular excipient and any accompanying elements of the composition will be adapted in accordance with the route and device used for administration. In some embodiments, the composition may also comprise or be accompanied with one or more other ingredients that facilitate the engraftment or functional mobilization of the cells. In some embodiments, suitable ingredients include, but are not limited to, matrix proteins that support or promote adhesion of the cells.

The methods provided herein may further comprise freezing the cells at liquid nitrogen temperatures and storing for long periods of time, being capable of use on thawing. For example, if frozen, the cells will usually be stored in any suitable medium, e.g. 10% DMSO, 20% FCS, 70% DMEM medium. Once thawed, the cells may be expanded by use of growth factors and/or feeder cells associated with progenitor cell proliferation and differentiation.

### Experimental

### EXAMPLE 1

The muscle microenvironment (niche) enables freshly isolated muscle stem cells (MuSCs) to contribute extensively to skeletal muscle regeneration when transplanted in mice. These include satellite cells and other cells that are capable of contributing to muscle (D. D. Cornelison et al. (2001) Dev Biol 239, 79; S. Fukada et al. (2004) Exp Cell Res 296, 245; D. Montarras et al. (2005) Science 309, 2064; S. Kuang et al. (2007) Cell 129, 999; M. Cerletti et al. (2008) Cell 134, 37; C. A. Collins et al. (2005) Cell 122, 289; A. Sacco et al. (2008) Nature 456, 502; R. I. Sherwood et al. (2004) Cell 119, 543; and Galvez et al. (2006) J Cell Biol. 174(2):231-43). In contrast, muscle stem cells grown on standard tissue culture plastic lose ‘sternness’ yielding progenitors with greatly diminished regenerative potential (D. Montarras et al. (2005) Science 309, 2064; A. Sacco et al. (2008) Nature 456, 502; Z. Qu-Petersen et al. (2002) J Cell Biol 157, 851) and therapeutic potency (E. Gussoni et al. (1992) Nature 356, 435). Biophysical properties such as matrix rigidity are known to alter the differentiation of cells in culture (M. P. Lutolf et al. (2009) Nature 462, 433). It is demonstrated below that elastic modulus plays a crucial role in muscle stem cell self-renewal and function in muscle regeneration. When MuSCs (satellite cells) are cultured in vitro on a substrate that mimics the rigidity of muscle tissue, they self-renew to generate stem cell progeny that can potently repair damaged muscle. Other cell types are also usefully cultured in the methods of the invention for increased viability and/or expansion.

### Materials and Methods

Animals. All animal protocols were approved by the Stanford University Administrative Panel on Laboratory Animal Care (APLAC) and experiments were performed in compliance with the institutional guidelines of Stanford University. Green Fluorescent Protein (GFP) transgenic mice (ubiquitous GFP expression) were a kind gift from I. L. Weissman (Stanford University, Stanford, Calif., USA). Animals ubiquitously expressing firefly Luciferase driven by the ATCB promoter (L2G85 strain) were a kind gift from C. H. Contag (Stanford University, Stanford, Calif., USA). Myf5-nLacZ transgenic mice were a kind gift from B. J. Wold (CalTech, Pasadena, Calif., USA). Pax7-ZsGreen transgenic mice were a kind gift from M. Kyba (University of Minnesota, Minneapolis, Minn.) (D. Bosnakovski et al. (2008) Stem Cells 26, 3194). All double transgenic animals were resultant of breeding the above strains followed by appropriate PCR based strategies to validate animal genotype. 6-8 week old NOD/SCID immunodeficient and C57BL/6 wild-type mice were purchased from the Jackson Laboratories.

Muscle stem cell isolation. Muscle stem cells were isolated as previously described (A. Sacco et al. (2008) Nature 456, 502). Briefly, murine tibialis anterior muscles were dissected and subjected to a gentle collagenase and dispase digestion. Non-muscle tissue was removed from single fiber cultures using a dissection microscope and the remaining cell suspension was filtered (70 μm pore size) and incubated with the following biotinylated antibodies against: CD45, CD11b, CD31 and Sca1 (BD Bioscience). Streptavidin microbeads (Miltenyi Biotech) were added to the cells to capture non-muscle cells types together with the following antibodies: α7integrin-Phycoerithrin (a kind gift from F. Rossi, Vancouver, Canada) and CD34-Alexa637 (BD Bioscience). After magnetic depletion of biotin-positive cells the negative population was fractionated twice to enrich for viable (PI−), CD34/α7integrin double positive cells by Fluorescence Activated Cell Sorting (FACS) on a Digital Vantage sterile cell sorter (Becton-Dickinson).

Hydrogel fabrication and ‘reactive’ microcontact printing PEG precursor gelatin and protein conjugation. Poly(ethylene glycol) PEG hydrogels were produced using an adaptation of a previously described mild and versatile chemistry (M. P. Lutolf, J. A. Hubbell (2003) Biomacromolecules 4, 713; M. P. Lutolf et al. (2009) Integrative Biology 1, 59). Precursors (4-arm 10 kDa PEG-SH, Laysan Bio and 8-arm 10 kDa PEG-VS), synthesized as previously described (M. Cordey, et al. (2008) Stem Cells 26, 2586), were dissolved at the specified solids concentration (w/v) in ultra-pure water (PEG-SH) or 0.3M triethanolamine (PEG-VS). Immediately after mixing precursors, the gel solution was pipetted between two hydrophobic glass slides (Sigmacote; Sigma) separated by a 1 mm spacer and incubated at 37° C. in a humidified unit to generate crosslinked gel networks. Laminin (Roche) was dialyzed into PBS and then pre-reacted with a 10-fold excess of Maleimide-PEG-SVA linker (Laysan Bio). After partial crosslinking of the gel networks, functionalized laminin was pipetted directly onto the surface of the gels and the crosslinking reaction was allowed to proceed for one hour at room temperature, resulting in covalent crosslinking of the laminin to the hydrogel surface. Hydrogel microwell arrays with laminin specifically microcontact printed to the bottom of microwells were generated as previously described (M. P. Lutolf et al. (2009) Integrative Biology 1, 59; M. Cordey, et al. (2008) Stem Cells 26, 2586). Briefly, hydrogel precursor solution was polymerized on a PDMS replica containing micropillars (the complementary array topography). Microwell masters were created by casting PDMS on silicon wafers containing the desired topography (generated using standard photolithography techniques). Laminin conjugated to a heterobifunctional PEG linker was patterned to the bottom of microwells by ‘inking’ linker functionalized laminin to the tips of the PDMS stamp containing hundreds of cylindrical micropillars, which was subsequently crosslinked to the hydrogel surface during polymerization. Stamp removal results in the generation of complementary microwell array topography. Prior to cell culture, PEG hydrogels were fixed to the bottom of sterile tissue culture dishes.

In order to control for surface chemistry and ligand density between hydrogel and plastic culture conditions, we cast a very thin hydrogel layer on plastic culture surfaces. Specifically, 1 μl of hydrogel precursor solution (4%) was pipetted into one well of a 24 well plate and immediately covered with a sterile 12 mm circular coverslip that was treated with SigmaCote (Sigma; to create a hydrophobic surface). After partial polymerization the coverslip was removed and prefunctionalized laminin was covalently attached to the surface as described above. Assuming the entire 1 μl of gel solution remained beneath the coverslip, we can conservatively calculate the thickness of the hydrogel to be ˜2 μm according to the following equation:

$$h = \frac{V}{π {r}^{2}} = \frac{1 μ L}{{π ( 1.2 cm )}^{2}} ≅ 2 μm$$

However, during this process much of the gel solution extends beyond the boundary of the coverslip, suggesting the hydrogel is thinner than our estimate predicts. According to literature, cells effectively sense the mechanical properties of the underlying plastic surface when the overlying gel surfaces are ≦0.5-1 μm in height (A. J. Engler et al. (2006) Cell 126, 677).

Timelapse acquisition. MuSCs were directly sorted into wells containing the above microwell surfaces. The plate was then placed in the environmental chamber of an inverted microscope (Zeiss Axiovert 200 or Zeiss PALM/AxioObserverZ1) equipped with a motorized stage. After cells were randomly distributed and trapped in microwells, the XYZ stage was programmed to repeatedly raster across the microwell array surface, acquiring phase contrast images at 10× magnification at multiple locations every 3 minutes (required to maintain lineage relationships) for a period of up to 7 days using Axiovision software. The resulting video images of the timelapse experiments were then processed using the Baxter algorithm (see below). Data presented here represents the behavior of muscle stem cells for a total of 3 days in culture.

‘Baxter algorithm’ data analysis. Timelapse analysis software was developed using Matlab (Mathwork, Inc., Natick, Mass.). Prior to analysis images were stabilized to correct for camera shake imparted by movement of the stage, using an ImageJ image stabilization plugin that was modified to be run from within our software. Microwell outlines were identified using a Sobel edge finder and a Hough transform for circular objects. The image background was removed to prevent stationary debris and microwell edges from being perceived as cells. The background image was computed by taking the median pixel intensity in the time dimension for every pixel in the image.

To find moving cells from brightfield images (3 minute acquisition rate required to maintain lineage relationships precludes the use of fluorescence due to photoxicity), Gaussian smoothing was applied to the absolute value of the background subtracted images. Then a modified, seeded watershed algorithm was used on the negative of this image. Seeds were put on local minima below a certain threshold, set by looking at the intensities of minima inside cells and in the background of representative images. To make the cell outlines as accurate as possible the watersheds were only filled to half the depth of the local minima. To avoid over-segmentation, regions that touched were then merged. For these regions, the probability that the region contained 0, 1 or more than 1 cell was computed, using multinomial logistic regression. The parameters used for these calculations were:

1) (area)/(average area in all regions of the sequence);

2) (area of convex hull)/(area);

3) (perimeter)/(radius of circle with the same area);

4) (distance from microwell center)/(microwell radius);

5) average absolute gradient component parallel to boundary;

6) average absolute gradient component perpendicular to boundary;

7) (mean intensity)−(mean intensity in all regions of the sequence).

Then cell trajectories were then added one by one, either by adding new cells in the first frame or by introducing cell divisions. During this process we also allowed swapping of line pairs in the cell trajectories. The construction and modification of these trajectories was done to try and maximize the utility function.

$$S = {\sum }_{c = 1}^{cellcount} [ log p ( {D}_{c} = 1 ) + {\sum }_{t = {t}_{first}}^{{t}_{last}} log p ( {T}_{c}^{t} = 1 ) ] + {\sum }_{b = 1}^{blobcount} log p ( {N}_{b} = {n}_{b} )$$

Here p(N_{b}=n_{b}) is the probability that blob b contains n_{b} cells, estimated using multinomial logistic regression as described above. If the hypothesized cell c divides, p(Dc=1) is the estimated probability that the division is correct. Otherwise p(Dc=1) is set to 1, so that nothing is subtracted from S. The division probability is estimated from the distance traveled by the cell during the 30 minutes preceding the division and the distance between the cell and the resulting daughter cells. p(T_{c}^{t}=1) is the probability that the movement cell c makes in frame t occurred, estimated from the distance between the cell positions in the two frames.

Algorithm mistakes occurred at a frequency of 1% and when desired were detected by watching the tracked movies and were rapidly corrected using a Matlab user interface. We observed no clear bias towards one specific type of tracking error. Some errors involved incorrect cell counts within segmented regions while others were due to inverted cell identities. Notably, mending an individual cell relationship error during manual correction often results in the repair of more than one trajectory error.

To establish the reduction in data analysis time, the amount of time it took to process video frames with different numbers of cells (up to the 16 cell stage) was determined by performing manual tracking using the Matlab interface we developed, compared to performing manual correction on automatically tracked videos. It was calculated that to manually track the entire data set (162 movies total) it would take 71 hours, while correcting all of the videos after they were automatically analyzed by the Baxter algorithm would take 7.5 hours.

To establish a measurement of error rate, we determined the number of cell trajectory mistakes in Baxter algorithm analyzed videos prior to manual correction. On average there were 10 trajectory errors per movie and the average error rate was 0.23 errors per cell per hour. Taking into consideration the 3 minute acquisition time we utilized, this was equivalent to an error rate of 1.2% and means that only about 1% of the lines in all cell trajectories were incorrect. Thus, manual correction is only needed if 99% accuracy does not suffice.

Proliferation rate modeling. To model the proliferation and death of cells in a culture condition we assume that the cells have a proliferation rate rₚ and a death rate r_{d}, and that these rates are not dependent on time. If the cell number as a function of time is c(t) we find that its derivative is

$$\frac{d c}{d t} = ( {r}_{p} - {r}_{d} ) c .$$

With a cell number of c₀ at t=0, we find that c(t)=c₀ exp((rₚ−r_{d})t). Given that we have n cell observations in total, nₚ observations of cell division and n_{d} observations of cell death, we can estimate rₚ and r_{d} as

$${r}_{p} = a \frac{{n}_{p}}{n} and {r}_{d} = a \frac{{n}_{d}}{n} ,$$

where a is the number of frames per time unit. Chi-square analysis was used to determine the statistical significance of the modeled proliferation and death rates. Random resampling was used to compare the mean cell counts of the raw data present in clones cultured on rigid or soft substrates at the end of the timelapse experiments. The resampling was repeated 10⁷ times and was performed with replacement. This method was used as we wanted to compare the means of two distributions which are not Gaussian. The two tailed t-test and the Wilcoxon rank sum test are more commonly used for similar purposes, but the West assumes Gaussian distributions and the Wilcoxon rank sum test does not specifically test for a difference in distribution means, therefore random resampling was the most appropriate test.

Muscle stem cell culture and transplantation. Immediately after FACS enrichment, muscle stem cells were transferred from FACS buffer into myogenic cell media (F10/DMEM (50/50)+15% FBS+2.5 ng/ml bFGF) and plated into tissue culture wells containing hydrogels (flat or patterned) or thin gel coated plastic (flat or patterned) as sparse cultures (i.e. 1000-2000 cells/well of a 24 well size plate). Media was replaced every 3 days.

The day of transplantation, NOD/SCID mice were anesthetized by intraperitoneal injection of Ketamine (2.4 mg/mouse) and Xylazine (240□g/mouse) and hindlimb irradiated as previously described (A. Sacco et al. (2008) Nature 456, 502). Freshly isolated or cultured muscle stem cells were counted and resuspended in 2.5% goat serum/1 mM EDTA in PBS in a 10□l cell suspension at the cell concentrations indicated within the text and subsequently injected intramuscularly into the tibialis anterior (TA) muscles of recipient mice. To activate muscle stem cells via local tissue injury, animals were anesthetized with isofluorane and a single 10 μl injection of notexin (10 μg/mL, Latoxan, France) was injected into recipient animal TA muscles.

Rheology. The shear modulus (G′) of both the gels and TA muscles was determined by small-amplitude oscillatory shear rheometry using an Anton Paar Physica MCR 301 (Anton Paar, Hertford Herts, UK). Parallel plates 7.95 mm in diameter were used for both hydrogels and TA muscles. In both cases, the samples were compressed (5-15%) with a normal force not exceeding 0.2 N. The storage (G′) modulus was measured at angular frequencies between 10 and 0.1 Hz and the final value was determined by taking the average of G′ values measured in the plateau region near 1 Hz and converting to Young's Modulus using the following equation: E=2(1+v)G′ and assuming a poisson ratio (v) of 0.5. All measurements were performed at low strain (5%) to ensure that the modulus was measured in the linear viscoelastic range.

ELISA assays. Ligand density on hydrogels was measured by performing a sandwich ELISA assay. Hydrogels with laminin crosslinked to the surface were fabricated as described above and then fixed to the bottom of a 96 well plate and incubated in 2.5 mg/ml of 2-aminoethanethiol (Sigma) to block any unreacted vinyl sulfone or thiol groups in the hydrogel network. To produce a calibration curve, various amounts of laminin were incubated in a Nunc Maxisorb plate (Nunc A/S, Rosklide Denmark) at 4° C. overnight. Both hydrogels and plastic were then blocked with 3% BSA to block unoccupied binding sites and subsequently washed 2 times with PBS containing 0.05% Tween-20 (PBST). Rabbit anti-mouse laminin (Immunology Consultants) was added to each well at 1:500 dilution and incubated at 37° C. for 2 hr. Plastic wells were washed 5 times with PBST. Hydrogel wells were also washed 5 times, but between each washing step were placed on a stirring table to ensure that unbound antibody would diffuse out of the hydrogel network. A secondary antibody conjugated to HRP (HRP-goat anti rabbit IgG FC, Zymed) was added at 1:1000 dilution to each well and incubated for 1 hr at 37° C. Washing was performed again in the same way as after primary antibody incubation. Finally, 100 μL of SureBlue Reserve TMB (KPL) was added to each well and the reaction was stopped using 0.6N sulfuric acid after 2-5 minutes, before saturation occurred. The absorbance at 450 nm was determined using a microplate reader (Biorad 450). The amount of protein bound to the hydrogels was determined by fitting a linear regression curve to the linear region of the calibration data, which contained values similar to those obtained on the hydrogels.

Hydrogel swelling analysis. The swelling capacity of hydrogels was determined by measuring the mass immediately after fabrication (mass,) and then again after 24 hours of equilibration in PBS (mass₂). These values were then used to determine the fold increase in mass following polymerization: mass₂/mass₁. A value equal to 1 indicates no swelling, while values over 1 indicate the degree of post polymerization swelling.

Bioluminescence imaging. We performed bioluminescence imaging using a Xenogen-100 device, as previously described (A. Sacco et al. (2008) Nature 456, 502; T. S. Wehrman et al. (2006) Nat Methods 3, 295). The system is comprised of a light-tight imaging chamber, a charge-coupled device (CCD) camera with a cryogenic refrigeration unit and the appropriate computer system (Living-Image Software; Xenogen). Animals were injected intraperitoneally with a 100 μl volume of luciferin diluted in PBS (0.1 mmol/Kg body weight, Xenogen). Immediately after injection, images were acquired every each minute for a total of 15 min and the data stored for subsequent analysis. Bioluminescence images were analyzed at 12 min after luciferin injection. Bioluminescence values below <10,000 photons/sec were set to exactly 10,000 photons/sec for esthetic purposes in figures.

Immunofluorescence and immunohistochemistry. Muscle tissues were prepared for histology as previously described (A. Sacco et al. (2008) Nature 456, 502; A. Sacco et al. (2005) J Cell Biol 171, 483). For immunofluorescence, rabbit anti-β-galactosidase (Abcam), rabbit anti-GFP (Molecular Probes), rat anti-laminin (Upstate Technologies) antibodies were used. For immunofluorescence of cultured cells or hydrogel-tethered molecules, we used previously described protocols (A. Sacco et al. (2008) Nature 456, 502; A. Sacco et al. (2005) J Cell Biol 171, 483) and the following antibodies: mouse anti-Pax7 (Developmental Mouse Hybridoma Bank) and rat anti-laminin (Upstate Technologies). Nuclei were visualized with Hoechst staining (Invitrogen). Antigens were visualized using the following secondary antibodies from Molecular Probes.

Images of muscle transverse sections and cell culture were acquired using an epi-fluorescent microscope (Axioplan2; Carl Zeiss MicroImaging, Inc.) equipped with a digital camera (ORCA-ER C4742-95; Hamamatsu Photonics) as previously described (A. Sacco et al. (2008) Nature 456, 502). Images of hydrogel staining were acquired using a laser-scanning confocal microscope (LSM510; Carl Zeiss MicroImaging, Inc.) as previously described (A. Sacco et al. (2008) Nature 456, 502). Images were composed in CorelDraw 12 and edited in Photoshop 5.5 (Adobe). Background was reduced using brightness and contrast adjustments, and color balance was performed to enhance colors. All modifications were applied to the whole image using Photoshop.

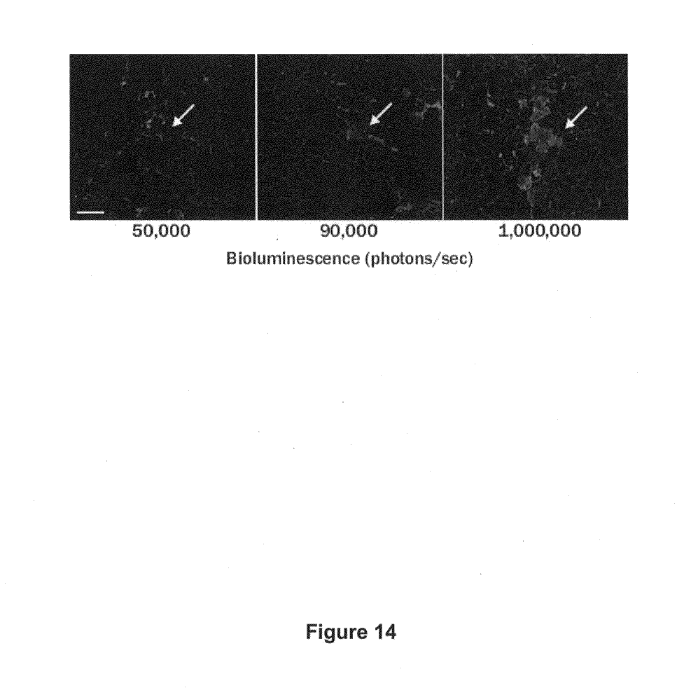

Pax7 immunofluorescence analysis. To analyze doublets for Pax7 expression, MuSCs were isolated from Pax7-ZsGreen transgenic mice and plated in both pliant and rigid microwell arrays fixed in the bottom of 24 well plates. Microwell arrays were imaged at 4 hours post-plating and fixed using paraformaldehyde at 48 hours post-plating. Single cells that had divided once to give a doublet were identified and imaged for ZsGreen (Pax7 expression) and Topro (nuclear stain) at 20× magnification using a laser-scanning confocal microscope (LSM510; Carl Zeiss MicroImaging, Inc). A Matlab script was used to process the confocal images as follows: every cell was manually outlined and the average fluorescent intensity was calculated by taking the sum of all pixel values within the segmented region and then dividing by the total area of the segmented region. Because the fluorescent intensity varied within each 24-well due to edge effects, signal was normalized by dividing the average ZsGreen intensity by the average Topro intensity. The normalized values for all single cells in the experiment were plotted on a scatterplot (FIG. 14) and used to establish a normalized ZsGreen threshold of 1.25; values higher than 1.25 represent positive Pax7 expression. This threshold was then applied and the data was analyzed for doublets in which both cells were Pax7 positive.

Statistical analysis. Culture experiments were performed in triplicate and in vivo experiments involved transplantation into at least 5 recipients. Graph averages are shown as ±SEM. Unless otherwise stated in the main text, legends or methods, the Student's t-test was used for comparisons between groups and assumed two-tailed distributions with significance set at <0.05.

### Results

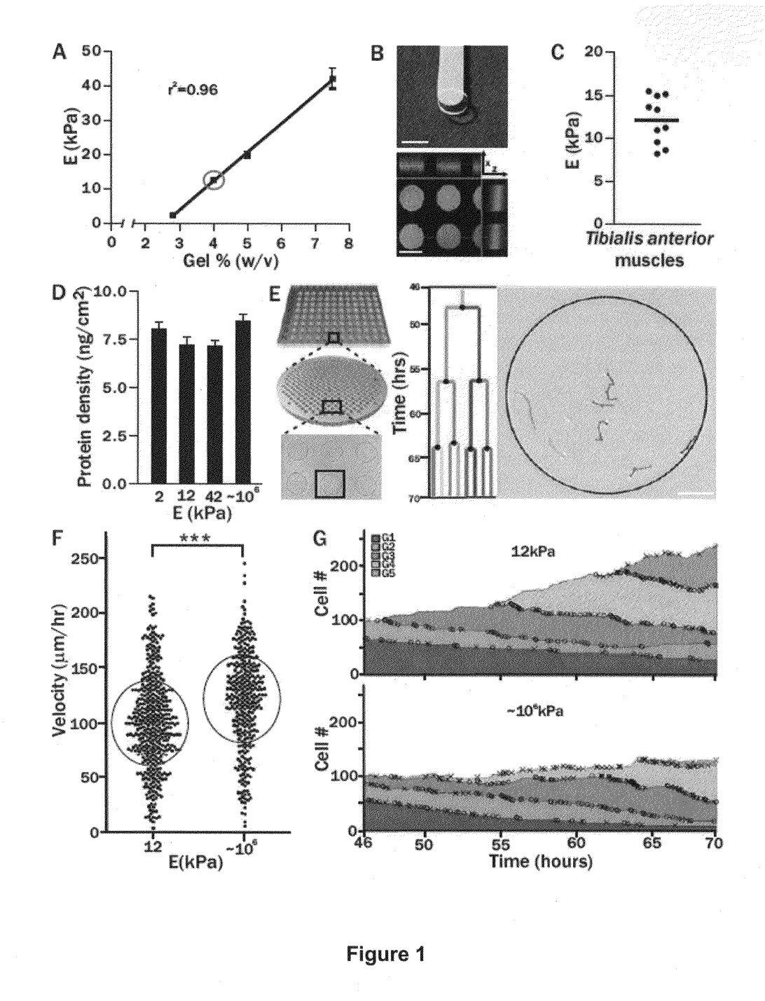

To recapitulate muscle rigidity and uncouple biophysical from biochemical effects, we engineered a tunable polyethylene glycol (PEG) hydrogel platform. By altering the percentage of PEG polymer in the precursor solution we produced hydrogels with a range of rigidities (FIGS. 1A and 1B top) including a formulation that mimics the elastic modulus of adult murine skeletal muscle (FIGS. 1A and 1C) (A. J. Engler et al. (2006) Cell 126, 677). Notably, polystyrene plastic traditionally used for cell culture, has an elastic modulus of ˜3 GPa, more than five orders of magnitude stiffer than skeletal muscle (W. D. Callister, Fundamentals of Materials Science and Engineering: An Interactive E-Text. John Wiley & Sons, Somerset, N.J., ed. 5th Edition, 2000). Laminin, a component of the native MuSC niche, was covalently crosslinked to the hydrogel network and used as an adhesion ligand (FIG. 1B bottom). To ensure that laminin density and surface chemistry remained consistent between hydrogel and plastic culture conditions, gels were generated that do not swell post-polymerization (FIGS. 1D; 5) and cast a thin layer of PEG hydrogel (≦1 μm) on the plastic surface, allowing MuSCs to sense the stiffness of the plastic beneath (see Supplemental Methods; FIG. 1D) (A. J. Engler et al. (2006) Cell 126, 677).

MuSCs were prospectively isolated (A. Sacco et al. (2008) Nature 456, 502) and analyzed at the single cell level on pliant or stiff culture surfaces patterned with arrays of microwells (FIG. 1E) (M. P. Lutolf et al. (2009) Integrative Biology 1, 59), because even when FACS enriched, stem cell populations are inherently heterogeneous (S. Kuang et al. (2007) Cell 129, 999; C. A. Collins et al. (2005) Cell 122, 289; A. Sacco et al. (2008) Nature 456, 502; R. I. Sherwood et al. (2004) Cell 119, 543; M. P. Lutolf et al. (2009) Integrative Biology 1, 59; L. Heslop et al. (2000) J Cell Sci 113 (Pt 12), 2299). Timelapse acquisition of hundreds of single stem cells is possible using microwell technology (M. P. Lutolf et al. (2009) Integrative Biology 1, 59); however, analysis of the resulting immense data sets remains a major challenge. To enable rapid analysis, a highly automated algorithm termed the ‘Baxter Algorithm’ (Methods) was developed, which in contrast to most commercially available software, is able to track lineage relationships over multiple cell divisions. This algorithm reduced data analysis time by ˜90% with a mere 1% error rate.

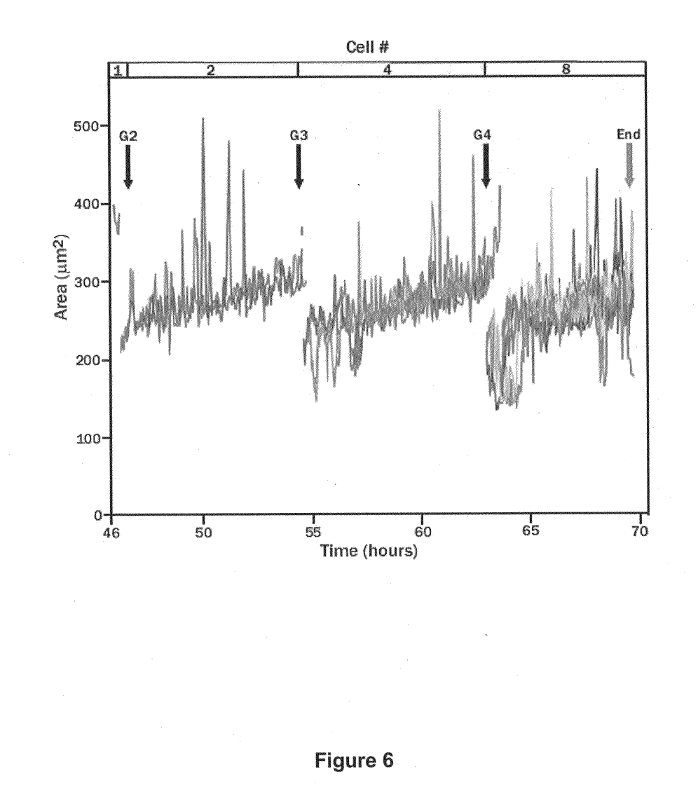

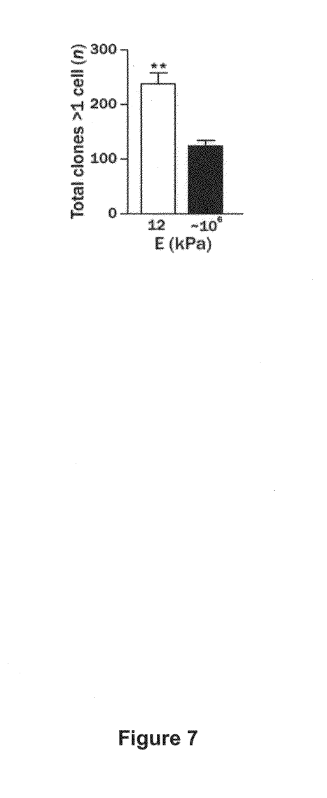

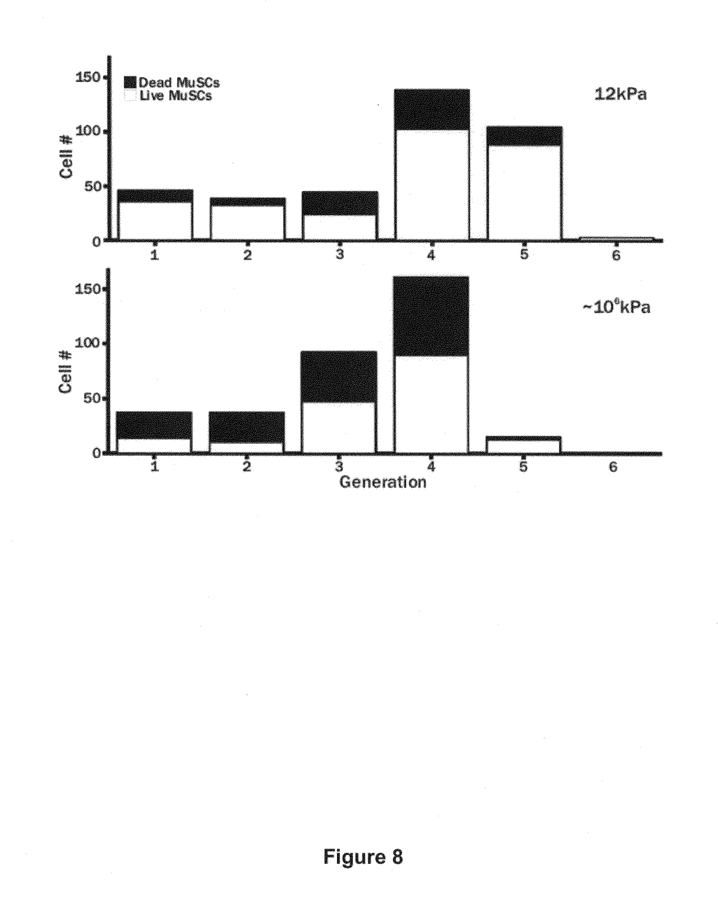

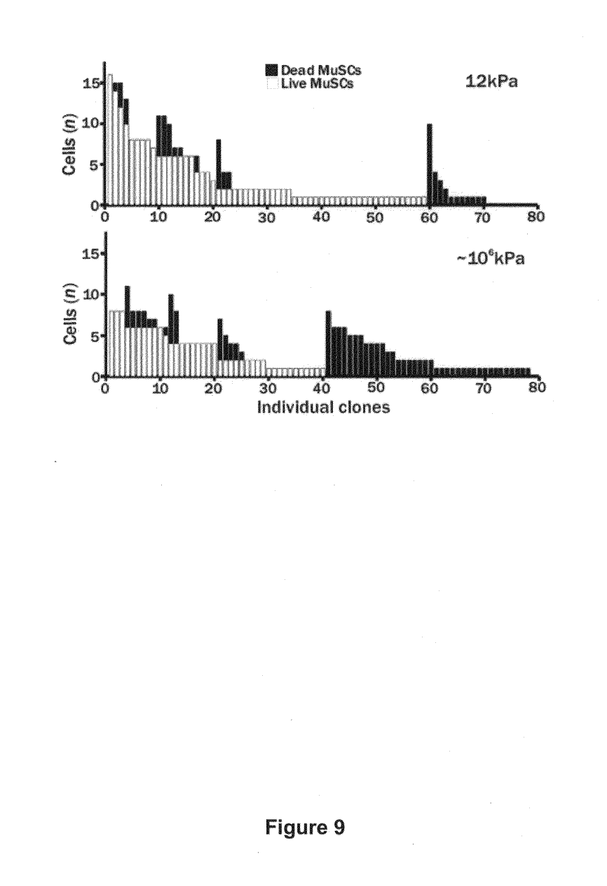

The genealogic history of clones derived from a single cell was established by timelapse acquisition and automated tracking (FIG. 1E). MuSC velocity, increased on stiff (120 μm/hr) compared to pliant (99 μm/hr) culture substrates, (FIG. 1F, p<0.0001), in agreement with previous reports investigating cell lines (R. J. Pelham, Jr., Y. Wang (1997) Proc Natl Acad Sci USA 94, 13661). In addition, cell area was observed to increase as cells duplicate their content, returning to initial cell area after mitosis, further validating our segmentation parameters (FIG. 6). MuSCs propagated on pliant hydrogel substrates do not undergo the ‘crisis’, or massive cell death previously reported in culture (Z. Qu-Petersen et al. (2002) J Cell Biol 157, 851; T. A. Partridge, in Basic cell culture protocols, J. W. a. W. Polard, J. M., Ed., Humana Press, Inc., Totowa, N.J., 1997, vol. 75, pp. 131-144). After one week of culture in soft microwells, twice as many cells give rise to clones as compared to cells cultured in rigid microwells (FIG. 7). Using the Baxter Algorithm the phenomenon was characterized at the clonal level. On rigid substrates, the overall cell number does not change over time because division is offset by death; however, on pliant substrates death is reduced and the total number of cells increases over time (FIGS. 1G, 8, and 9). In both conditions, death is not sudden; indeed it is independent of time and cell division number (FIGS. 1G and 8). This data demonstrates that culture on soft substrates augments MuSC survival.

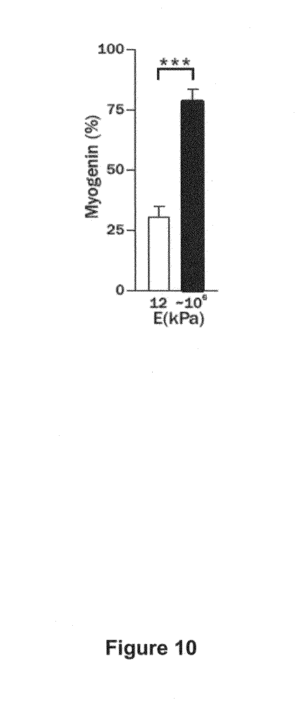

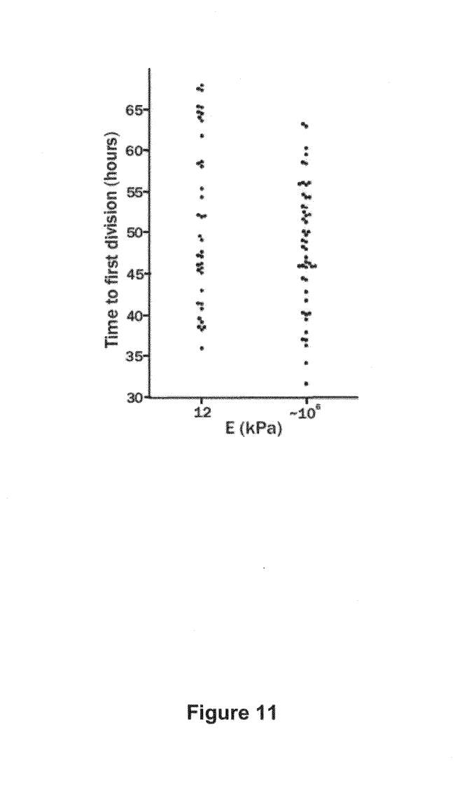

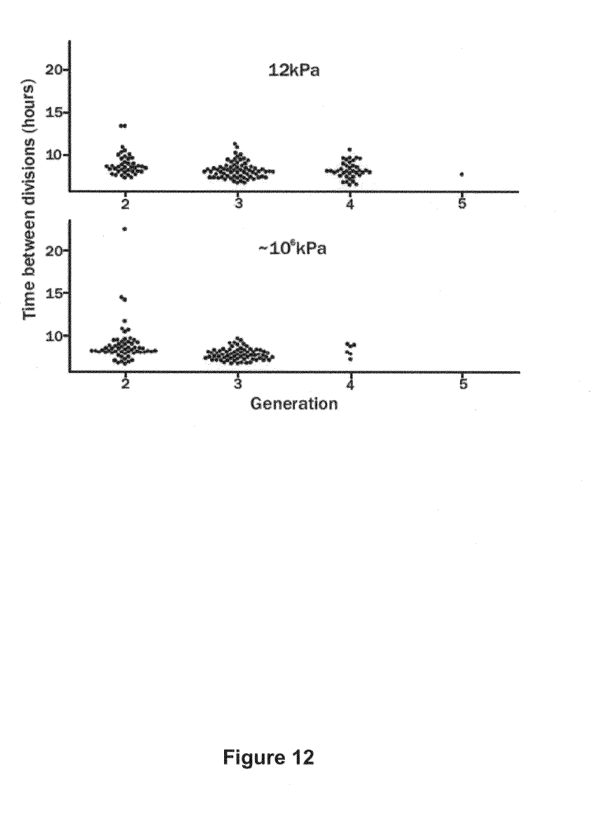

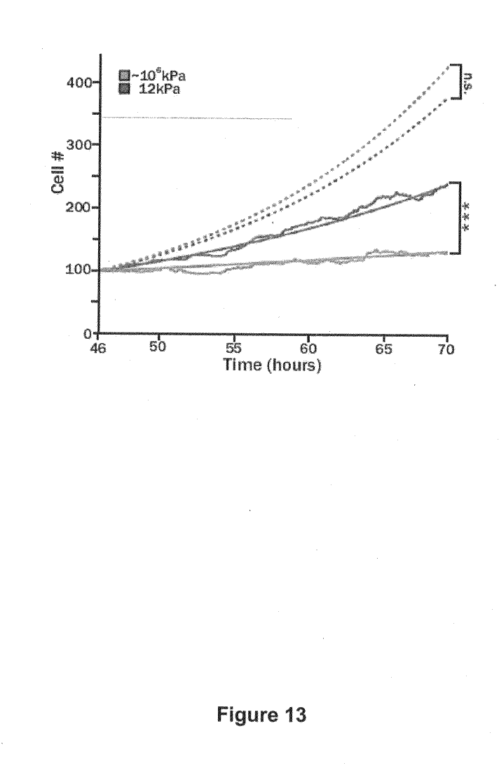

Substrate rigidity also impacts gene expression, suggesting that MuSC sternness is retained on soft surfaces. MuSCs cultured for one week on soft substrates give rise to 3-fold fewer cells that express Myogenin, a myogenic transcription factor expressed by differentiated MuSCs, than MuSCs cultured on rigid substrates (FIG. 10). Timelapse analysis excludes the possibility that gene expression differences are due to differences in cell division, as we observe no statistically significant difference in MuSC time to first division (FIG. 11) or time between divisions (FIG. 12). In addition, division rate is not different on pliant compared to rigid substrates (FIG. 13 and Methods). These in vitro studies demonstrate that substrate rigidity has no effect on cell division rate in culture but prevents differentiation and leads to increased cell numbers by enhancing viability.

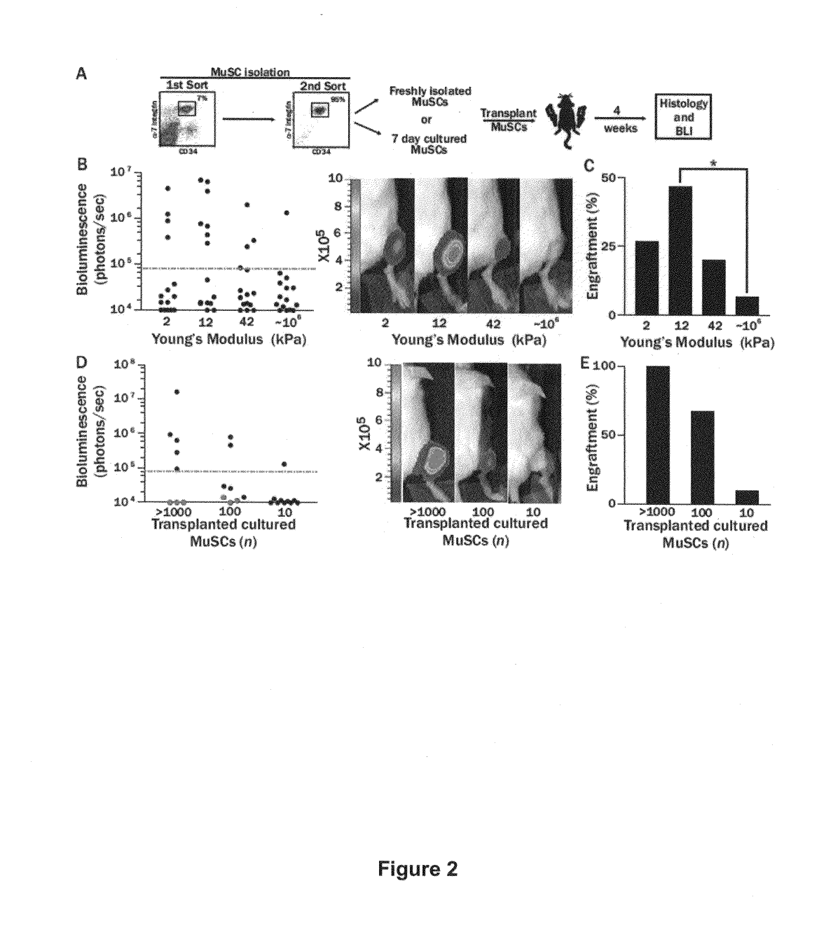

To establish definitively that MuSCs cultured on pliant substrates retain sternness, their function was assessed in vivo. MuSCs were isolated from mice constitutively expressing firefly luciferase (Fluc) (Y. A. Cao et al. (2004) Proc Natl Acad Sci USA 101, 221) and green fluorescent protein (GFP) and cultured for 7 days. Cells were harvested, counted and cultured MuSC progeny were transplanted into the tibialis anterior (TA) muscles of immunodeficient mice depleted of endogenous MuSCs by 18Gy leg-irradiation (L. Heslop et al. (2000) J Cell Sci 113 (Pt 12), 2299; S. Wakeford et al. (1991) Muscle Nerve 14, 42). We assayed donor cell behavior quantitatively over time by non-invasive in vivo bioluminescence imaging (BLI) of luciferase activity, which correlates with cell number (FIG. 2A) (A. Sacco et al. (2008) Nature 456, 502). The engraftment threshold that was set as the bioluminescence value corresponding with histological detection of donor derived myofibers (FIG. 14). This in vivo functional assay is critical for validating the sternness of cultured MuSCs.

In vivo functional assays show that MuSCs cultured on substrates matching the physiological modulus of muscle tissue most potently retain sternness. Hydrogel substrates were tuned to mimic the endogenous in vivo mechanical properties of brain, muscle and cartilage (2, 12, or 42 kPa) compared to polystyrene plastic (˜10⁶ kPa). In agreement with previous reports (D. Montarras et al. (2005) Science 309, 2064; A. Sacco et al. (2008) Nature 456, 502; Z. Qu-Petersen et al. (2002) J Cell Biol 157, 851), we observe markedly reduced engraftment from MuSCs cultured on plastic (FIG. 2B). Strikingly, the highest bioluminescent signals are obtained from mice transplanted with MuSCs cultured on the most pliant hydrogels, whereas both the extent and rate of engraftment are decreased on the stiffest culture substrates (FIG. 2B). Notably, the culture substrate that recapitulates skeletal muscle elasticity (12 kPa) is the only condition that leads to a statistically significant increase in the percentage of mice with MuSC engraftment compared to tissue culture plastic (FIG. 2C).

To determine the proportion of cultured cells with engraftment potential, we cultured MuSCs for one week on either soft or rigid substrates and then performed dilution analysis. None of the mice transplanted with stem cells cultured on a rigid plastic substrate exhibit a BLI signal above the threshold of detection (FIG. 2D; red circles). By contrast, engraftment occurs with 100% frequency when ≧1000 hydrogelcultured (12 kPa) stem cells are transplanted (FIG. 2D; black circles), similar to freshly isolated cells (A. Sacco et al. (2008) Nature 456, 502). Moreover, 10% of mice transplanted with only 10 hydrogel cultured cells exhibit engraftment (FIG. 2E), on par with transplantations of 10 freshly isolated cells (16%) (A. Sacco et al. (2008) Nature 456, 502).

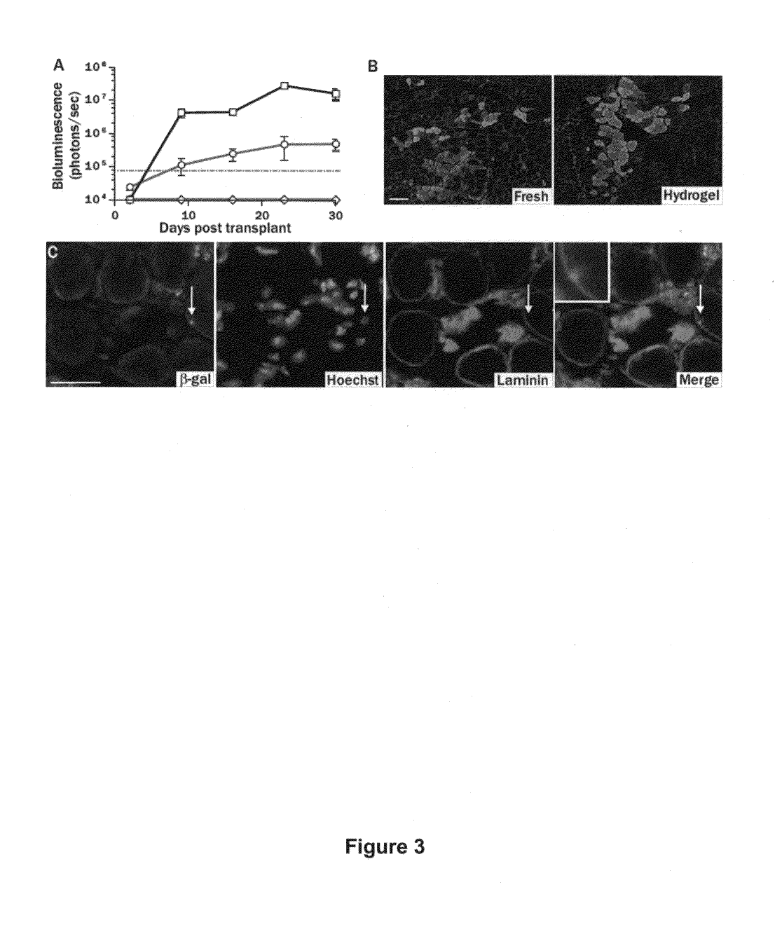

MuSCs cultured on a substrate that mimics muscle tissue exhibit dynamic proliferative behavior similar to freshly isolated MuSCs when transplanted in vivo. Both cell populations undergo a period of extensive proliferation that ultimately plateaus and stabilizes when homeostasis is achieved (FIG. 3A). Histology identifies GFP+ myofibers resulting from regeneration in animals transplanted with MuSCs that were freshly isolated or cultured on soft substrates (FIG. 3B). While the engraftment rate of freshly isolated MuSCs (A. Sacco et al. (2008) Nature 456, 502) and those grown on soft substrates is comparable (FIG. 2C), the extent of engraftment from cultured MuSCs is not as robust as that of freshly isolated cells (FIG. 3A), suggesting that exposure to additional biochemical cues in vitro may be required to recapitulate other key niche components necessary for maximal function in vivo.

MuSCs cultured on soft substrates can home to their native satellite cell niche upon transplantation into muscle, a defining characteristic of freshly isolated MuSCs (D. Montarras et al. (2005) Science 309, 2064; A. Sacco et al. (2008) Nature 456, 502). MuSCs isolated from mice expressing LacZ under the regulation of the Myf5 promoter (S. Tajbakhsh et al. (1996) Dev Dyn 206, 291) were cultured on soft hydrogel and subsequently transplanted into mice. Prior to harvesting tissues, recipient muscles were damaged with notexin (J. B. Harris, M. A. Johnson (1978) Clin Exp Pharmacol Physiol 5, 587), which activates MuSCs and upregulates expression of the myogenic transcription factor Myf5 (A. Sacco et al. (2008) Nature 456, 502; S. Tajbakhsh et al. (1996) Dev Dyn 206, 291). Histological analysis of β-galactosidase (β-gal) staining reveals that, like freshly isolated cells (A. Sacco et al. (2008) Nature 456, 502), transplanted hydrogel cultured cells expressing Myf5 are found in the satellite cell niche, beneath the basal lamina and atop myofibers (FIG. 3C).

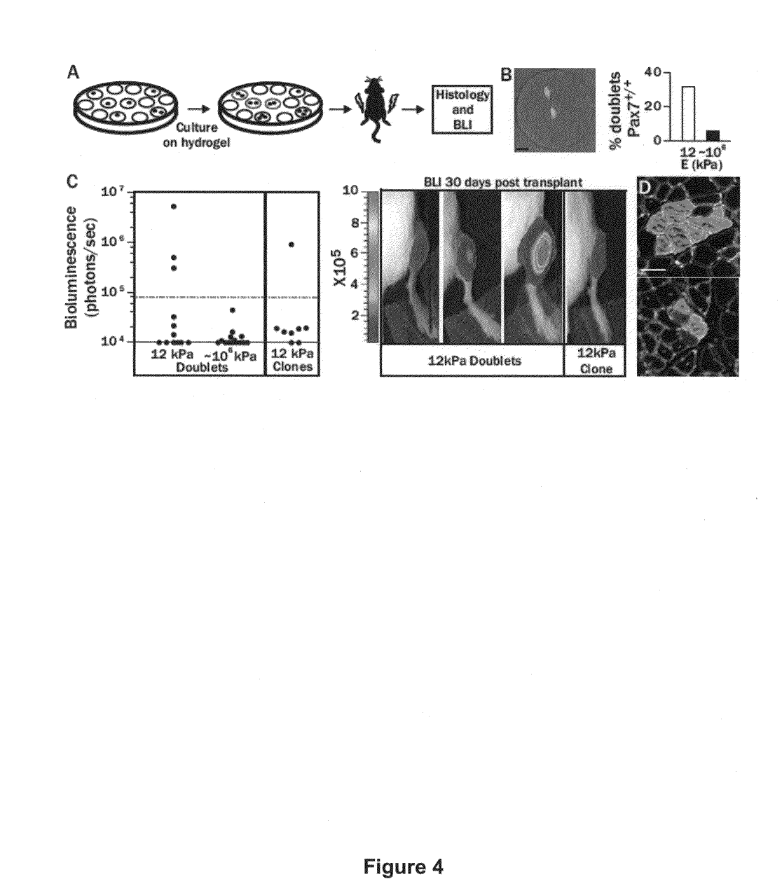

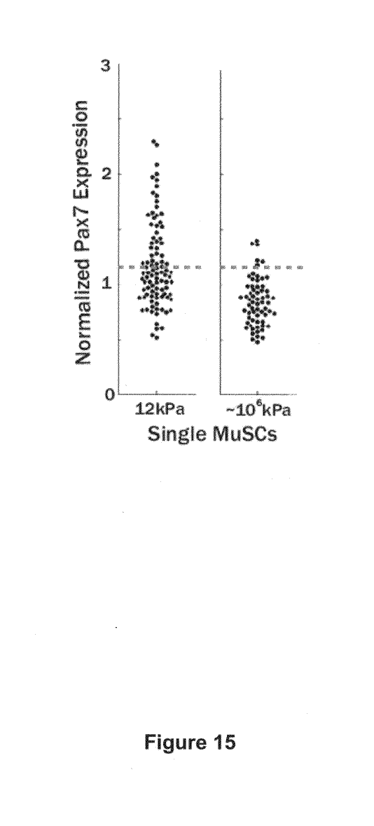

One of the defining characteristics of stem cells is their ability to make more copies of themselves upon division, or self-renew. Several elegant in vitro approaches have provided strong evidence that MuSC asymmetric and symmetric divisions occur in culture, consistent with self-renewal (S. Kuang et al. (2007) Cell 129, 999; R. Abou Khalil et al. (2009) Cell Stem Cell 5, 298; V. Shinin et al. (2006) Nat Cell Biol 8, 677; M. J. Conboy et al. (2007) PLoS Biol 5, e102; I. M. Conboy, T. A. Rando (2002) Dev Cell 3, 397; F. Le Grand et al. (2009) Cell Stem Cell 4, 535; P. S. Zammit et al. (2004) J Cell Biol 166, 347). Our in vitro analysis of MuSC gene expression suggests that a pliant substrate supports self-renewal. We observe that 32% of doublets (two cells that arose from a single cell division) in pliant microwells are positive for the MuSC marker Pax7 (FIGS. 4A, 4B and 15). In contrast, only 6% of doublets in plastic microwells have this gene expression pattern, suggesting that a pliant substrate enables MuSC expansion. Although gene expression data are suggestive, an in vivo functional assay is necessary to conclude definitively that a self-renewal division event occurred in culture.

We show conclusively that stem cell self-renewal occurs using an in vivo functional assay. The transplantation of MuSCs at a population level demonstrates engraftment (FIGS. 2 and 3), but does not definitively show that self-renewal divisions occurred in culture, because the population could include non-dividing cells which maintained stem cell properties. Accordingly, in this experiment, we plated MuSCs in hydrogel microwell arrays and obtained images immediately after plating and 2-3 days after culture to identify microwells that contained only one doublet. Doublets from 5 microwells were picked and pooled using a micromanipulator and 10 cells total transplanted per mouse (FIG. 4A). A detectable BLI signal indicates engraftment resulting from a self-renewal division event that must have occurred in at least one of the five transplanted doublets. Strikingly, 25% (3/12) of animals transplanted with doublets cultured on soft substrates demonstrate detectable engraftment (FIG. 4C) and contribution to regenerating myofibers (FIG. 4D, top), providing in vivo functional evidence that MuSC self-renewal division events occur in culture on pliant substrates. In contrast, doublets grown on rigid plastic microwells never exhibit engraftment following transplantation (FIG. 4C; 0/14), indicating that their regenerative potential is rapidly lost.

MuSC self-renewal on pliant hydrogel occurs even after multiple divisions. We transplanted clones that arose from a single cell which underwent. 3-5 divisions. Remarkably, 12% (1/8) of animals transplanted with a single clone show engraftment, demonstrating that MuSC self-renewal capacity is retained on pliant substrates even after multiple divisions (FIGS. 4C and 4D, bottom).

Here we provide novel insight into the potency of tissue rigidity, a biophysical property of the skeletal muscle microenvironment, on stem cell fate regulation. Using a novel single cell tracking algorithm to interrogate MuSC behaviors at the single cell level, we demonstrate that soft substrates enhance MuSC survival, prevent differentiation and promote sternness. Functional assays in mice demonstrate conclusively that pliant substrates permit MuSC self-renewal in culture. While the underlying mechanisms remain to be elucidated, we hypothesize that decreased rigidity preserves sternness by altering cell shape, resulting in cytoskeletal rearrangements and altered signaling as shown for cell lines (R. J. Pelham, Jr., Y. Wang (1997) Proc Natl Acad Sci USA 94, 13661). Despite the remarkable retention of sternness in response to a single parameter, rigidity, we anticipate further enhancement of sternness through incorporation of additional biochemical cues into our reductionist platform. Studies employing biomimetic culture platforms, such as described here for MuSCs, will broadly impact stem cell studies by facilitating in vitro propagation while maintaining sternness and the capacity to regenerate tissues, a critical step towards the development of cell-based therapies.

### EXAMPLE 2

### Method of Gel Fabrication

Sulfhydryl (SH) groups on 4-armed PEG (PEG-SH) are reacted with vinyl sulfone (VS) groups on 8-arm PEG (Polyethylene Glycol Vinyl Sulfone, or PEG-VS). The resulting preparation results in the formation of non-swelling PEG (NS-PEG) hydrogels. The method converts the end groups of polyethylene glycol (PEG) to vinyl sulfone. A large excess of divinyl sulfone is used to react with the end groups, yielding an intact vinyl sulfone moiety at the end of each PEG chain. The reaction mechanism is shown in scheme. 1.

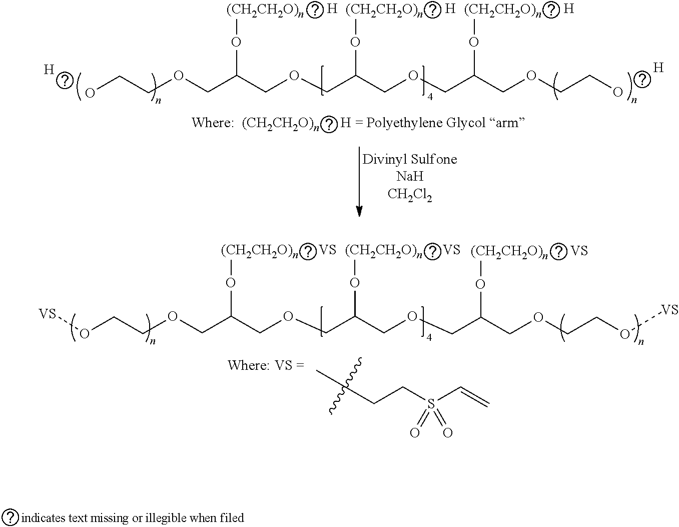

A strong base, in this case sodium hydride, is used to deprotonate the diol group, leaving O— as a strong nucleophile to react across the double bond of the divinyl sulfone.

| | | |
|---|---|---|
| Weight of PEG | 5 | g |
| MW of PEG | 10000 | Da |
| Number of arms | 8 | |
| Concentration of DVS | 0.67 | M |
| Molar Excess Sodium Hydride | 15 | |
| Quantities of reagents | | |
| PEG | 5 | g |
| Divinyl Sulfone | 9.5 | g |
| Sodium Hydride dispersion | 2.4 | g |
| Total Reaction Volume | 120 | mL |
| Volume DCM in flask | 90 | mL |
| Volume DCM in funnel | 20 | mL |

The rate of reaction is extremely fast, probably near instantaneous and definitely <5 mins. % Conversion depends on several factors. “Accessibility” of end groups which in turn depends on molecular weight of PEG chains, solvent, temperature, concentration of divinyl sulfone, concentration of sodium hydride. Gelation via crosslinking will occur if the concentration of unreacted PEG in the mixture is too high.

Divinyl sulfone has trace hydroquinone which has two diols and therefore will polymerize with the vinyl sulfone when base is present. The hydroquinone must be removed to prevent contamination of the final product. Deprotonated hydroquinone is yellow so you can monitor the presence of hydroquinone by looking for the characteristic bright yellow. Low molecular weight PEG is difficult to react. This is probably because dichloromethane is a very good solvent for high molecular weight PEG, leading to high end group accessibility. PEG undergoes a transition around 2000 Da, when solvent/polymer interactions change dramatically. Dichloromethane solvent quality appears to be much lower for PEG chains<2000, leading to lower end group accessibility. For example, all other conditions being equal, end group conversion is >95% for 5000 Da PEG chains and ˜35% for 1250 Da PEG chains.

Protocol: dry PEG by azeotropic distillation in toluene. Dissolve PEG in 250 mL round bottom flask in ˜100 mL of toluene. Rotovap off solvent (I usually do it at 65 C). Remove as much solvent as possible. Set up a 3 neck, 250 mL round bottom flask under argon. The reaction will need to be heated to 35 C so set up the flask in a water or mineral oil bath on a heated stir plate. Set the temperature to 35 C. Wash the sodium hydride to remove the mineral oil. Add the 40% dispersion of sodium hydride to the bottom of the flask. Add pentane and stir for ˜1 min. Turn the stir plate off and allow the sodium hydride to settle to the bottom of the flask. Pour off the supernatant and neutralize with isopropyl alcohol. Repeat the wash procedure 3× to remove all residual mineral oil. Add 50 mL of dry dichloromethane to the sodium hydride in the round bottom flask. Add divinyl sulfone to the reaction flask. Mix the divinyl sulfone with 40 mL of dry dichloromethane. Prepare a 10 mL syringe with inhibitor remover packing. Put glass wool in the syringe and then load it with inhibitor remover. Clamp the syringe so it will drip into the round bottom flask containing the sodium hydride. Add the divinyl sulfone/dichloromethane solution by pouring it through the inhibitor remover in the syringe and allowing it to drip into the round bottom flask. Use the plunger to remove as much of the solution from the syringe as possible. Place a 50 mL addition funnel into the middle neck of the round bottom flask. The entire apparatus should now be under argon. Make sure the solution is being well mixed and heated to 35 C. Dissolve the dry PEG in 20 mL of dichloromethane. Add this PEG solution to the addition funnel. Drip the PEG solution slowly into the stirred solution of divinyl sulfone/sodium hydride in dichloromethane. The drip rate should be relatively slow ˜1 drop/sec.

Purification. To neutralize the reaction, add glacial acetic acid dropwise until hydrogel evolution stops and the solution becomes clear. Filter the solution and discard the solids (these are salts from neutralization and are insoluble in dichloromethane). Concentrate by removing the dichloromethane via rotovap. Do not use heat at this step or in any subsequent steps because it will degrade the product. Put 1 L of ethyl ether in a large beaker on a stir plate. Stir the ether and add the concentrated PEG solution dropwise. The PEG solution has to be very concentrated and viscous. Cover the beaker and put in the freezer for ˜20 minutes. Pour off the supernatant and wash the remaining PEG crystals with excess ether by rinsing with clean ether and pouring off the supernatant. Collect the PEG crystals, dissolve in a small volume of dichloromethane (˜5-10 mL) and recrystalize in ethyl ether as describe above. Repeat the recrystallization once more. The final product should be collected and stored under vacuum to remove all residual ether before being aliquoted and stored under argon for long term storage at −20 C or −80 C.

Analysis is performed using a range of methods including but not limited to MALDI mass spectrometry (MALDI-MS), NMR, swelling analysis, and rheometry. Here we show examples of analysis using MALDI-MS and NMR. The protocol for preparing a sample of PEG-VS for MALDI-MS analysis is as follows: dissolve 1 mg PEG material in 10 uL deionized water. Combine 1 uL of PEG solution with 9 uL of 50:50 wt/wt acetonitrile:water with 0.1% Trifluoroacetic acid containing 10 mg of dihydrobenzoic acid (DHB). Mix well and plate on stainless steel 100-well MALDI plate. Typical MALDI-MS spectra characterizing the starting material and product of the above synthesis protocol are shown FIGS. 3 and 4.

The product is highly polydisperse; i.e. there are PEG-VS precursors of varying size (3 kDa to 10 kDa molar mass) and shape; we believe this is important for creating a sufficiently strong and tangled polymer network that results in the non-swelling character of these hydrogels.

NMR analysis of PEG-VS product shows the NMR spectrum of the starting material, 8-arm, 40 kDa PEG. After functionalization with divinyl sulfone, peaks from the hydrogen atoms present on the attached vinyl sulfone moiety are observed in the spectrum. Integration of the PEG backbone peak (3.64 ppm) can be compared to peak integrations of hydrogen atoms on the functional moiety to determine end group conversion. Integrations of the PEG backbone peak is set such that 100% functionalization corresponds to H1 and H2 peaks integrating to 2 and H3, H4, H5 peaks integrating to 1.

Making gels of different stiffnesses. Typically after a batch of precursor has been fabricated, a calibration step is performed, in which PEG-SH and PEG-VS are mixed with solvent at various concentrations and cured. The elasticities of the resulting hydrogels are then measured using a rheometer, for example, allowing a calibration curve to be constructed.

Differences from other hydrogels and hydrogel fabrication methods. The gel produced by the above process is almost non-swelling, which may be due to the polydisperse nature of the PEG-VS precursor; i.e., the precursor has a wide range of sizes and shapes.

The invention depends on the matching of the elasticity of an artificial substrate to the elasticity found in a physiological tissue with which the cells cultured on the substrate naturally come into contact in vivo, or to which they are adapted, or to which they will be delivered. Using the hydrogel fabricated using the method described above, hydrogel with the following elasticities were synthesized: 2, 12, 42, and 10⁶ kPa. Muscle elasticity is approximately 12 kPa. Muscle stem cells cultured on hydrogels of 12 kPa maintained their ability to contribute to muscle regeneration better than muscle stem cells cultured on other elasticities, as shown in Example 1.

### EXAMPLE 3

### ART Techniques

ART techniques to which the invention applies include but are not limited to: Egg collection, in which oocytes are gathered from the ovaries, typically through a needle inserted through the vaginal wall. The invention provides methods and systems to receive the oocytes in a container with an elastic substrate.

Sperm collection and processing, in which sperm are donated by a male and either frozen for future use or used fresh. The invention provides methods and systems to receive sperm in a container with one or more elastic substrates during collection, for example sperm collection containers containing elastic substrates.

Sperm activation, in which sperm are cultured for a few hours in an environment which activates them, meaning it causes them to become more motile. The invention provides methods and systems to culture sperm in contact with one or more elastic substrates during activation, for example Petri dishes containing elastic substrates.

Banking, in which sperm, oocytes, follicles, or embryos are frozen, stored, and thawed. The invention provides methods and systems for containing these cells in contact with one or more elastic substrates during banking, for example cryovials containing elastic substrates.

IVF, which is typically performed by co-culturing oocytes or cumulus-oocyte complexes with sperm. The invention provides methods and systems including culture dishes and wells to contain the oocytes and/or sperm in contact with one or more elastic substrates during IVF, for example Petri dishes with elastic substrates.

Embryo stripping, in which the cumulus oophorous is removed from the cumulus-oocyte complex, leaving the bare embryo, by pipetting. The invention provides tools including pipets coated with an elastic substrate for performing embryo stripping, and a container comprising one or more elastic substrates for containing the cells during stripping.

ICSI, in which a single sperm in injected via a glass needle into an oocyte. The invention provides methods and systems to contain and handle the sperm, oocytes, and cumulus-oocyte complexes in contact with one or more elastic, substrates during ICSI. For example, the holding pipet and the injection needle may be coated with an elastic substrate to protect the cells, and the procedure may be performed in a container which includes one or more elastic substrates. The holding pipet may have an elastic substrate so that the oocyte is not pulled too far into the lumen. The injection needle or holding pipet may also have an elastic plug so that the embryo or sperm are not pulled too far into the lumen of the pipet or needle. The holding pipet may be replaced by a holding container such as a modified Petri dish, in which an elastic water-permeable substrate on the bottom of the dish covers one or more suction ports underneath the substrate, optionally with one or more channels through the substrate, or wells on top of the substrate, so that the oocyte is held by suction in place on top of the channel or well.

Culture of the conceptus to allow it to develop to a stage appropriate for transfer to the uterus or fallopian tubes, typically at the embryo or blastocyst stage. The invention provides methods and systems for culturing the conceptus in contact with one or more elastic substrates, for example Petri dishes with elastic substrates.

Assisted zona hatching, in which the zona pellucida surrounding the oocyte is mechanically or chemically punctured or weakened to allow the oocyte to more easily exit the zona pellucida. The invention can be used to contain the oocyte in contact with elastic substrates during the procedure, and additionally to hold the oocyte using a holding pipet coated with an elastic substrate.

Transfer of gametes, a zygote, embryo, or blastocyst to the uterus or a fallopian tube of the female recipient using a catheter. The invention provides methods and tools for holding the gametes or concepti as they are being transferred, including a catheter coated with an elastic substrate, and a catheter coated with an elastic substrate and also containing an elastic plug to prevent the embryo or blastocyst from being drawn too far up into the catheter. The invention also provides elastic substrates shaped so that they contain the embryo or blastocyst so that the substrate and embryo or blastocyst may be transferred together to the uterus by a suitable transfer device, after which fluid may be forced through the substrate to dislodge the embryo or blastocyst, after which the substrate may be withdrawn from the uterus leaving the embryo or blastocyst in the uterus.

Preimplantation genetic diagnosis (PGD), in which one or more cells are collected from an embryo and analyzed, for example for genetic markers of disease. The invention provides holding pipets coated with elastic substrates and containers such as Petri dishes containing elastic substrates to contact the embryo during collection of material for analysis.

Gamete intrafallopian transfer (GIFT) and zygote intrafallopian transfer (ZIFT), in which oocytes, sperm, or embryos are placed into the fallopian tubes. The invention provides containers with elastic substrates for culturing the gametes or zygotes before transfer and transfer tools coated with elastic substrates for transferring the gametes or zygotes.

The invention is also applicable to other ART procedures, and to variations on the ART procedures described above, which vary depending on the state of the sperm and oocytes, geographic location, and other variables. The invention is also applicable to tools and techniques used in the transfer, culture, and manipulation of gametes and concepti during the generation of animals including transgenic animals, and cloning of animals. The invention is not limited to mammals, but may be used for other species, for example insect species, as well.

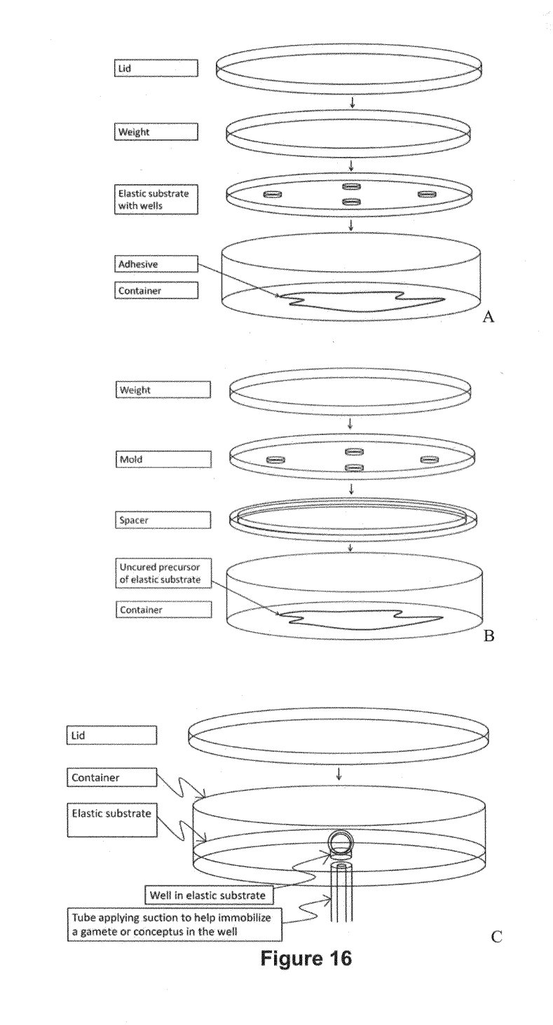

The invention also provides methods for making culture systems with elastic substrates for use in the above ART procedures, for example as shown in FIGS. 16A-C. FIG. 16A shows a method for making a culture dish with an elastic substrate. In this version, the invention comprises an adhesive which bonds the elastic substrate to the container. A weight holds the elastic substrate against the container while the adhesive is curing. FIG. 16B, containers may be coated with an elastic substrate by placing uncured substrate in contact with the container and then forming it with a mold while it cures and adheres to the container. In a variation of the invention, a region of negative pressure may be created under the elastic substrate to assist in holding gametes or concepti in contact with the substrate during manipulations. In a further variation, a region of negative pressure may be near a well in the elastic substrate, for example, by having a tube connect to the bottom of the container at a location near a well, an reducing the pressure in the tube, as shown in FIG. 16C. A holding dish for holding gametes or concepti during manipulations such as ICSI, cytoplasmic transfer, or harvesting of cells for PGD. A region of negative pressure is created near a well in the elastic substrate by a tube connected to the bottom of the container. The well may be slightly smaller than the gamete or conceptus so that it sits projecting above the surface of the elastic substrate, as shown in the inset.

Components of the cumulus-oocyte complex are. 1: oocyte; 2: perivitelline space (PS); 3: polar body; 4: zona pellucida (ZP); 5: cell of the corona radiata (and also of the cumulus oophorus, which includes the corona radiata); 6: extracellular matrix of the cumulus oophorous, consisting of abundant hyaluronic acid. Immediately after fertilization the ZP hardens, and then over the next several days it softens and thins until the developing blastocyst hatches out of it. The cumulus cells stay attached to the zona pellucida for at least 3 days after ovulation, including after fertilization. The invention applies to the culture of both cumulus-oocyte complexes and cumulus-embryo complexes.

The perivitelline space (PS) is a fluid-filled space which surrounds the oolema (plasma membrane of the oocytes), and is in turn bounded by the approximately spherical shell-shaped zona pellucida (ZP). Typically part of the oolema is in direct contact with the SP, and part of it in direct contact with the PS. Immediately following ovulation the PS is very thin and it enlarges during the cortical reaction following fertilization, thereby helping to prevent polyspermic fertilization. During passage through the oviduct the PS absorbs proteins secreted by ciliated epithelial cells lining the ampulla, but not the isthmus, of the fallopian tube. 3 Thus the viscosity of the PS may change over time. Since the PS comprises a fluid, the elastic modulus of the PS is probably low, and probably <500 Pa, however it may also be heterogeneous, and have regions, such as those where polar bodies are located, which are stiffer.

The Zona pellucida (ZP) is a layer of glycoproteins which surrounds the plasma membrane of the oocyte and conceptus until the blastocyst hatches from the ZP approximately 4-6 days after fertilization. At ovulation the human ZP is approximately 17-18 microns thick, or approximately 10% of the approximately 150 micron diameter of the secondary oocyte. The ZP is composed of three or four glycoproteins depending on the species. In humans these are ZP1, ZP2, ZP3, and ZPB.5 In mice ZP2 and ZP3 form most of the ZP mass and ZP1 acts to tether ZP2 and ZP3 molecules. The glycoproteins are produced exclusively by the oocyte during oocyte maturation in the follicle. The ZP is highly porous and permeable to antibodies and viruses. The ZP can be dissolved in conditions which do not necessarily break covalent bonds, including low pH or ionic strength buffers or increased temperature.

The ZP has several important functions: it reduces the chances of interspecies fertilization, reduces the chances of polyspermy due to penetration of the oocyte by multiple sperm, and reduces the chances of fraternal twins due to breaking apart of the embryo during cleavage stages. The ZP may also contribute to specification of the blastocyst axis.7 Since the ZP shape determines orientation of the blastocyst axis, perturbation of the ZP, for example its deformation due to flattening of one side due to resting on a hard culture surface, may affect the ability of cells in the embryo to orient themselves properly with respect to each other. This hypothesis may be consistent with the finding that embryos with harder ZP in vitro are more likely to implant; possibly the harder ZP is deformed less allowing the embryo to develop more normally. Thus, culture on soft surfaces as the current invention provides may facilitate successful pregnancy by allowing the ZP to maintain a more ovoid shape. This would be consistent with the theory that the reason why regular movement of embryos during culture on hard surfaces increases the chances of successful pregnancy is that it prevents the embryo from being permanently deformed due to resting on a hard surface for a prolonged period in a specific orientation.

Sperm bind to carbohydrate or peptide groups on ZP3 causing the acrosome reaction in which sperm expose enzymes to the ZP, resulting in a hole of approximately the size of the sperm head being created in the ZP thus allowing the sperm to reach the oocyte membrane to which it fuses. Sperm fusion immediately changes the membrane polarization of the oocyte and increases intracellular calcium causing the cortical reaction in which the contents of vesicles called cortical granules are released from the oocyte into the perivitelline space. The cortical granules contain peroxidases, proteases, and glycosidases which cleave carbohydrates on ZP3 preventing sperm binding to the ZP3; cleave sperm receptors on the oocyte membrane preventing sperm binding to the membrane; and partially hydrolyze ZP2 to ZP2f. This is believed to cause the ZP to “harden”, a term used with two meanings in the ZP literature: first, the modulus of elasticity of the ZP increases from about 7-18 kPa to about 13-42 kPa, depending on the species, measurement conditions, and techniques; 8,9,10 and second, the ZP becomes more resistant to dissolution in conditions of low pH or ionic strength or elevated temperature and to protease digestion. The mechanism by which hydrolysis of ZP2 to ZP2f causes hardening of both sorts is not clear but is believed to be at least partly due to an increased density of interaction points between ZP fibrils.

Before fertilization in vitro spontaneous hardening of the ZP can occur due to release of cortical granules. Removal of cumulus cells increases this hardening. This effect is reduced by culture in low oxygen and certain media components such as the protease inhibitor fetuin.

In vivo, the ZP has an elasticity between 5-20 kPa before fertilization; 10-45 kPa immediately after fertilization; and then thins and softens over several days following fertilization until the blastocyst hatches out of the ZP as early as E4.14 This softening and thinning is believed to be facilitated by enzymes in the reproductive tract such as fetuin.

The enzymes which weaken the ZP in vivo are not present in vitro and thus while in vivo the blastocyst can be free of the ZP by E4, in vitro it is often necessary to create a slit or hole in the ZP to enable the blastocyst to hatch.

The ZP has different properties in different species, as shown below. Devices and containers of the present invention utilize a substrate with elasticities that mimic the elasticities of the ZP in the appropriate species.

| Species | Conclusions |
|---|---|
| Mouse | GV, MII, PN, 2cell, 4cell, 8cell, M and EB was 22.8 +/− 10.4 kPa (n = 30), 8.26 +/− |
| | 5.22 kPa (n = 74), 22.3 +/− 10.5 kPa (n = 66), 13.8 +/− 3.54 kPa (n = 41), |
| | 12.6 +/− 3.34 kPa (n = 19), 5.97 +/− 4.97 kPa (n = 6), 1.88 +/− 1.34 kPa (n = |
| | 8) and 3.39 +/− 1.86 kPa (n = 4), respectively. Modulus of mouse oocyte and |
| | post-fertilzation zona pellucida is 17.9 kPa and 42.2 kPa, respectively |
| Human | Zona pellucida modulus 7 kPa before fertilization and 13 kPa after, |
| | respectively, by one method; 2.5 kPa and 4.5 kPa, respectively, by another |
| | method |
| Cow | “ZPs of embryos generated in vivo are significantly harder than those of |
| | embryos generated in vitro at each stage” |
| Hamster | Escape of blastocysts from zona pellucida is slower and morphologically |
| | different in vitro than in vivo; may be due to uterine contribution, since zona |
| | pellucida in vivo shows thinning. |
| Pig | Culture medium affects zona pellucida “hardness” (digestibility, not elastic |
| | modulus) |
| Multiple | “Mouse (Larman et al., 2006), pig (Coy et al., 2008), sheep (Huneau et al., |
| species | 1994), horse (Dell'Aquila et al., 1999) and cattle (Iwamoto et al., 1999) oocytes |
| | have been shown to undergo spontaneous zona pellucida hardening when |
| | exposed to in vitro culture conditions.” |

Corona radiata (CR) comprises two or three layers of cumulus cells surrounding the zona pellucida. The cumulus cells are embedded in a gel consisting largely of hyaluronic acid. To reach the zona pellucida sperm secrete hyaluronidase which digests the hyaluronic acid. Many sperm are required to secrete enough hyaluronidase to weaken the corona radiata sufficiently for a single sperm to penetrate to the ZP.

Cumulus oophorous (CO) comprises cells surrounding the ZP and embedded in a gel of hyaluronic acid, and indeed the CO can be stripped from the secondary oocyte and zona pellucida by digestion with hyaluronidase. The inner layer of the CO is the CR. The CO surrounds the egg and ZP starting in the follicle and for at least three days after fertilization.19 As the cumulus oocyte complex (COC) moves from the ruptured follicle to the fallopian tube, it adheres to cilia which roll the COC, compacting the CO, until the COC enters the ostium of the infundibulum.

Outside the CO, the conceptus is surrounded by a series of macroenvironments as it migrates from the ovary to the endometrium of the uterus, including vaginal wall; uterine wall; endometrium; finestraie; infundibulum; ampulla of the fallopian tube; isthmus of the fallopian tube; ovarian follicle; ovary; cervix. The oviduct comprises the infundibulum, ampulla, and isthmus of the fallopian tube. The cumulus oocyte complex hatches from the ovary and enters the infundibulum of the fallopian tube where it continues to move under the influence of waving ciliated endothelia lining the tube into the ampulla which is the most common site of fertilization. After fertilization the conceptus continues to move towards the uterus and approximately 5 days after fertilization has become a blastocyst and has hatched from the zona pellucida. The blastocyst then implants in the endometrium.

The oviduct is the lumen connecting the infundibulum of the fallopian tube to the uterus. It varies in diameter, widest at the infundibulum, narrower at the ampulla, and narrower still at the isthmus just before it opens on the uterus which contains its destination, the endometrium. The invention provides containers with elastic substrates with elasticities which approximate all of these structures and others in the reproductive tract, and with shapes mimicking their surface shapes, for example the folds of the ampulla or the folds and crypts of the endometrium. Here we describe two of them in more detail.

The ampulla is the most common site of fertilization, and is filled with highly folded tissue layers with channels between them. Thus in one variation of the invention, sperm and egg are cultured in a container with an elastic substrate mimicking the elasticity of the ampulla. In another variation this substrate is folded on its surface. In a further variation, the substrate is made to move in a way which mimics movements of the ampulla under the influence of the muscles which surround the fallopian tube. In a further variation, the elastic substrate releases molecules which are released.

The endometrium is the lining of the uterus, and is the structure into which the blastocyst implants at around day 5-6 after ovulation. Therefore in one variation of the invention, the invention provides a culture vessel with an elastic substrate with elasticity similar to that of the endometrium, to condition the embryo to adapt to such an elasticity.

In addition to elasticity, the invention may mimic other properties of the environments encountered by gametes or concepti in vivo. In one variation the elastic substrate is actively stretched, twisted, or otherwise deformed during culture to mimic deformation of environments of tissues in the reproductive tract. This may stimulate gametes or concepti, and may enhance the transport of oxygen and nutrients through the substrate, mimicking delivery of oxygen and nutrients from blood vessels. In another variation, the elastic substrate may be shaken, rocked, or otherwise moved in a way which mimics movements of tissues which gametes or oocytes encounter in vivo. In another variation, the elastic substrate may be coated with molecules which mimic molecules which gametes or concepti encounter in vivo; such molecules may be covalently or non-covalently associated with the elastic substrate.

### EXAMPLE 4

### Propagation of Mouse Pancreatic β-Islet Cells for the Treatment of Diabetes

Using a Streptozotocin-induced pancreas regeneration model, we mated two transgenic mice: Tg(RIP-Cre/ESR1)Ydor mice (Rat-Insulin-Promoter directed Cre which specifically targets the beta-cells, see Beta Cell Biology Consortium) and the floxed Rb/ARF mice. Streptozotocin (STZ) is an alkylating agent that selectively affects pancreatic islet β-cells, inducing diabetes in mice. STZ triggers an inflammatory process that causes the further loss of β-cell activity and results in insulin deficiency and hyperglycemia, thus providing a mouse model that closely resembles type 1 diabetes mellitus (T1DM), both with respect to pathogenesis and morphologic changes. Conventionally, STZ is administrated as a single high dose to cause complete β-cell necrosis and diabetes within 48 hr (Kolb, 1987). A modified protocol involving administering low-dose STZ to mice on 5 consecutive days was used to produce a delayed onset of hyperglycemia and to reduce the rapid, toxic activity of the drug. This multiple, low-dose STZ approach partially damages pancreatic islets, allowing the remaining beta-cells to regenerate the damaged pancreas. The pancreas of STZ-induced Rb/p19 −/− mice were analyzed for the following parameters indicative of pancreas regeneration: (1) proliferation index of β-cells relative to α-cells, (2) differentiation of β-cells to insulin producing cells, (3) size and number of islet cells, and (4) glucose levels (functional test).

Additionally, murine islet cells were isolated and cultured on a hydrogel substrate to induce cell cycle reentry. These cells were characterized for proliferation. We determined that the hydrogel substrate was critical to the survival of islets. The ability to culture the islets allows the additional screening of soluble or tethered niche factors, which may further enhance the re-entry of the cell cycle. Islet cells cultured on the hydrogel substrate may be transplanted into an individual in order to treat a diabetic condition.

### EXAMPLE 5

### Hydrogel Embryo Culture 1

### Experimental Details:

Female mice were induced to hyperovulate and then mated with male mice. One-cell embryos were harvested immediately after observance of copulation ‘plug’ and were plated within a ligand-free hydrogel microwell (500 um) arrays with plastic (10⁶ kPa—plastic or 42 kPa—hydrogel) and phase images were acquired daily to assess viability and embryonic development stage. Gels were covered in mineral oil and cultured under hypoxic conditions.

### Experimental Conclusions:

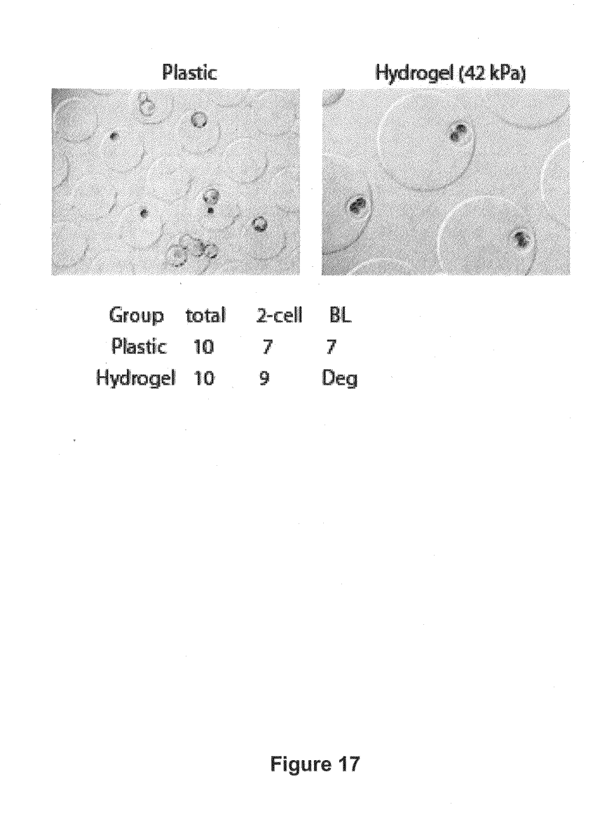

A total of 10 fertilized embryos were assessed in each experimental condition. 7/10 embryos on the 10⁶ kPa plastic survived to the 2-cell stage while 9/10 embryos on 42 kPa hydrogel survived to the 2-cell stage. However, analysis 2 days later when blastocysts (BL) are expected revealed substantial degradation (deg) on the hydrogel while those on plastic survived (FIG. 17).

### EXAMPLE 6

### Hydrogel Embryo Culture 2

Experimental Details

Female mice were induced to hyperovulate and then mated with male mice. One-cell embryos were harvested immediately after observance of copulation ‘plug’. Embryos were plated within ligand-free hydrogel microwell (150 um) arrays with plastic (10⁶ kPa—TCP or 20 kPa—hydrogel or 42 kPa—hydrogel) and phase images were acquired daily to assess viability and embryonic development stage. Gels were covered in mineral oil and cultured under hypoxic conditions.

Experimental Conclusions

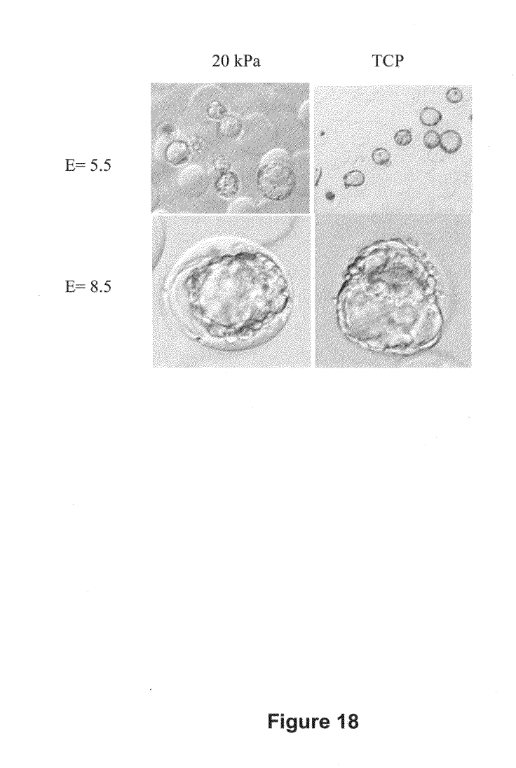

More single cell embryos survived to cleave into 2-cell embryos on 20 kPa (13/15) and TCP (12/15) compared to culture on 42 kPa (6/15). Hence, embryo survival is sensitive to substrate rigidity. 100% survival might be predicted to occur on hydrogels softer than 20 kPa (FIG. 18 and Table 1). Notably, of those embryos that survived to the 2-cell stage, a higher proportion of embryos developed into robust blastocysts (BL) on 20 kPa hydrogel (11/13) than on plastic (TCP; 9/12) suggesting 20 kPa soft hydrogel might promote embryonic development. Importantly, embryos expanded up to E8.5 in culture when cultured upon TCP or 20 kPA hydrogel, which suggests that hydrogel might provide a unique setting to study late stage embryo development. Previous techniques only permit study up to day 4.5 for murine and 7 days for human embryogenesis studies in culture.

TABLE 1

| Group | Total | 2-Cell | BL |
|---|---|---|---|
| TCP | 15 | 12 (80%) | 9 (75%) |
| 20 kPA | 15 | 13 (86.7%) | 11 (84.6%) |
| 42 kPA | 15 | 6 (40%) | 5 (83.3%) |

A closer look at the data reveals that the only culture condition that permitted proper development growth in culture is the 20 kPa hydrogel. At Day5.5 in culture embryos should have hatched from the blastocyst stage (hBL)). When cultured at 20 kPa all embryos that survived reached hBL at the expected D5.5 timepoint. Both TCP and42 kPa contained incidences when embryos reached the expanded blastocyst stage (exBL), but did not hatch. This suggests a soft hydrogel substrate is optimal to support proper embryonic development in culture (Table 2).

TABLE 2

| Group | D0.5 | D1.5 | D2.5 | D4.5 | D5.5 |
|---|---|---|---|---|---|
| TCP | 15 | 2Cell-11 | 8Cell-9; | BL-10; | hBL-7; |
| | | | 3Cell-3 | Morula-2 | exBL-2 |
| 20 kPa | 15 | 2Cell-13 | 8Cell-13 | BL-11; | hBL-11 |
| | | | | Morula-2 | |
| 42 kPa | 15 | 2Cell-6 | 8Cell-4; | BL-2; | hBL-1; |
| | | | 4Cell-2 | Morula-4 | exBL-4 |

Typically murine embryos are cultured in groups in a drop of media on a plastic dish, covered in mineral oil and cultured under hypoxic conditions. The group culture seems to be critical for maintaining murine embryo survival, but still, survival to 2-cell stage doesn't usually extend above 50%. In the studies a plastic only control was not used—a ‘plastic’ control contained a thin layer of hydrogel that permitted the embryos to ‘sense’ the stiffness of the plastic below, but to also benefit from the diffusion characteristics of the hydrogel. It should be noted that in the experiments embryos were not cultured in groups, but as single embryos and we observed substantial survival in many conditions. We predict that the presence of hydrogel in all conditions is responsible for this overall enhanced survival due to enhanced diffusion. Our data also clearly show a preference at 20 kPa for proper development. Our data also suggest that hydrogel permits studies of long term development for the first time in culture.

1. A method for in vitro cell culture, comprising:
seeding at least one cell in a container comprising a hydrogel substrate, wherein the substrate has an elasticity that is matched to the elasticity of the tissue from which the cell is derived; and
maintaining the cell for a period of time in vitro.

2. The method of claim 1, wherein the cell is said cell is a stem cell, primary cell, transdifferentiated cell, dedifferentiated cell, reprogrammed cell, multipotent cell, gamete, or pluripotent cell.

3. The method of claim 2, wherein the stem cell is a somatic tissue stem cell.

4. The method of claim 2 wherein the stem cell is an embryonic stem cell, an iPS cell, or a fertilized oocyte.

5. The method of claim 2, wherein the cells are expanded in culture.

6. The method of claim 1, further comprising a step of transplanting the cells into a recipient animal.

7. A method for the ex vivo manipulation of stem cells or reproductive cells, the method comprising:
manipulating said stem cells or reproductive cells in a container or device comprising an elastic substrate, wherein the substrate has an elasticity that mimics the elasticity of a native microenvironment of the stem cell or the reproductive cell.

8. The method of claim 7, wherein the stem cell or reproductive cell is a mammalian cell.

9. The method of claim 8, wherein manipulating includes fertilization in vitro.

10. The method of claim 7, wherein manipulating comprises culture of the stem cell or the reproductive cell.

11. The method of claim 10, comprising the step of transferring the stem cell or the reproductive cell to a mammalian recipient following said manipulating step.

12. The method of claim 7, wherein said reproductive cell is an embryonic stem cell, spermatozoon, egg, gamete, gametocyte, spermatocyte, oocyte, zygote, or fertilized oocyte.

13. The method of claim 7, wherein the number of said stem cells, said reproductive cells, or derivatives thereof, is expanded in culture.

14. The method of claim 7, wherein said stem cells or said reproductive cells are transferred in a device comprising the elastic substrate.

15. The method of claim 7, wherein said stem cells or said reproductive cells are frozen in a vial comprising the elastic substrate.

16. The method of claim 7, wherein said stem cells or said reproductive cells are thawed in a container comprising the elastic substrate.

17. The method of claim 7, wherein the elastic substrate is a hydrogel.

18. The method of claim 17, wherein the hydrogel is a non-swelling hydrogel.

19. The method of claim 17, wherein the hydrogel is polymerized from highly polydisperse precursors.

20. The method of claim 17, wherein the hydrogel is comprised of polyethylene glycol (PEG).

21. The method of claim 7, wherein the substrate has an elasticity of from about 0.01 to about 2000 kPa.

22. The method claim 17, wherein the hydrogel comprises a polymer selected from the group consisting of: a poly(ethylene glycol), a polyaliphatic polyurethane, a polyether polyurethane, a polyester polyurethane, a polyethylene copolymer, a polyamide, a polyvinyl alcohol, a polypropylene glycol, a polytetramethylene oxide, a polyvinyl pyrrolidone, a polyacrylamide, a poly(hydroxyethyl acrylate), a poly(hydroxyethyl methacrylate), poly(glycolic acid), poly(DL-lactic-co-glycolic acid), polyhydroxyalkanoate, poly(4-hydroxybutirate), sulfonated polymers, polygluconic acid, poly(acrylic acid), polyphosphazenes, polysaccharides, proteins, collagen, elastin, alginate, fibrin, fibronectin, laminin, hyaluronic acid, and polypeptide sequences cleavable by proteases including matrix metalloproteases.

23. The method of claim 22, wherein said hydrogel comprises polyethylene glycol sulfhydryl (PEG-SH) and polyethylene glycol vinyl sulfone (PEG-VS).

24. The method of claim 7, wherein the substrate further comprises at least one polypeptide component, biomolecule component, or chemical component.

25. The method of claim 24, wherein the polypeptide, biomolecule or chemical is soluble, or crosslinked to the hydrogel.

26. The method of claim 25, wherein the polypeptide is a structural protein.

27. The method of claim 24, wherein the biomolecule is a growth factor or cytokine.

28. The method of any one of claim 1, further comprising a step of monitoring the cells by an automated or semi-automated process.

29. The method of claim 13, further comprising a step of transplanting expanded reproductive cells, stem cells, or derivatives thereof, into a recipient animal.

30. The method of claim 7, further comprising contacting the cells with a candidate agent to determine the effect on cell phenotype, growth or expansion.

31. The method of claim 30, wherein the candidate agent is a cell, drug, genetic agent biomolecule or chemical.

32. A culture system for use with the method of claim 1 or claim 7.

33. A system for analysis for tracking individual cells in culture; comprising
a computer configured with software for tracking individual cells; by the steps comprising:
applying a modified, seeded watershed algorithm to background subtracted images, where the probability that a region contains 0, 1 or more than 1 cell is computed, using multinomial logistic regression, using the parameters 1) (area)/(average area in all regions of the sequence); 2) (area of convex hull)/(area); 3) (perimeter)/(radius of circle with the same area); 4) (distance from microwell center)/(microwell radius); 5) average absolute gradient component parallel to boundary; 6) average absolute gradient component perpendicular to boundary; and 7) (mean intensity)−(mean intensity in all regions of the sequence);
adding cell trajectories and
determining the probability for cell movement and division.

34. A hydrogel for the culture of cells, wherein said hydrogel comprises a first polymer and a second polymer, wherein the elasticity of said hydrogel mimics the elasticity of a native microenvironment of a cell and wherein said first polymer is modified to contain a vinyl sulfone group or said second polymer is modified to contain a sulfhydryl group.

35. The hydrogel of claim 34, wherein said first polymer and said second polymer are selected from the group consisting of: a poly(ethylene glycol), a polyaliphatic polyurethane, a polyether polyurethane, a polyester polyurethane, a polyethylene copolymer, a polyamide, a polyvinyl alcohol, a polypropylene glycol, a polytetramethylene oxide, a polyvinyl pyrrolidone, a polyacrylamide, a poly(hydroxyethyl acrylate), a poly(hydroxyethyl methacrylate), poly(glycolic acid), poly(DL-lactic-co-glycolic acid), polyhydroxyalkanoate, poly(4-hydroxybutirate), sulfonated polymers, polygluconic acid, poly(acrylic acid), polyphosphazenes, polysaccharides, proteins, collagen, elastin, alginate, fibrin, fibronectin, laminin, hyaluronic acid, and polypeptide sequences cleavable by proteases.

36. The hydrogel of claim 35, wherein said hydrogel is produced by reacting said first polymer with said second polymer.

37. The hydrogel of claim 36, wherein said first polymer is PEG-sulfhydryl (PEG-SH).

38. The hydrogel of claim 37, wherein said second polymer is PEG vinyl sulfone (PEG-VS).

39. The hydrogel of claim 34, wherein said hydrogel is polymerized from highly polydisperse precursors.

40. The hydrogel of claim 34, wherein said hydrogel has an elasticity of from about 0.01 to about 2000 kPa.

41. A hydrogel for the culture of cells, wherein said hydrogel comprises a first polyethylene glycol (PEG) and a second PEG, wherein the elasticity of said hydrogel mimics the elasticity of a native microenvironment of a cell and wherein said first PEG is modified and wherein said second PEG is modified in a different manner.

42. The hydrogel of claim 41, wherein said first PEG and said second PEG are covalently modified.

43. The hydrogel of claim 34 or 41, wherein said hydrogel is a non-swelling hydrogel.

44. A cell culture substrate comprising an elastic substrate; wherein said elastic substrate comprises at least two regions, a first region and a second region, wherein said first region has an elasticity that differs from the elasticity of said second region.

45. The cell culture substrate of claim 44, wherein said elastic substrate is a hydrogel.

46. The cell culture substrate of claim 44, wherein said elasticity of said first region is two-fold higher than said elasticity of said second region.

47. A composition comprising an elastic substrate and a reproductive cell or a stem cell.

48. The composition of claim 47, wherein said elastic substrate is said hydrogel of any one of claims 34 or 41.

49. The composition of claim 47, wherein said reproductive cell is a gamete, embryonic stem cell, spermatozoon, egg, gamete, gametocyte, spermatocyte, oocyte, zygote, or fertilized oocyte or progeny thereof.

50. A method of culturing cells, the method comprising:
(a) culturing a cell on an elastic substrate, thereby producing a cell culture; and
(b) varying the elasticity of the substrate during the course of said culturing of said cell.

51. The method of claim 50, wherein said cells are cultured for an hour or more during said varying of the elasticity of the substrate.

52. The method of claim 50, wherein said varying of the elasticity of said substrate is accomplished by changing the ionic strength or pH of the culture media, light exposure, bond formation or breakage, polymeric composition, or temperature of said cell culture substrate or adding or removing biomolecules, chemicals, or catalysts to said cell culture substrate.

53. The method of claim 50, wherein said elasticity of said substrate increases by 10%, over 1 minute to six months.

54. The method of claim 50, wherein said elasticity of said substrate decreases by 10%, over 1 minute to six months.

55. The method of claim 50, wherein said elastic substrate is a hydrogel.

56. The method of claim 50, wherein the hydrogel comprises a polymer selected from the group comprising polyethylene glycol, polyaliphatic polyurethane, polyether polyurethane, polyester polyurethane, polyethylene copolymer, polyamide, polyvinyl alcohol, polypropylene glycol, polytetramethylene oxide, polyvinyl pyrrolidone, polyacrylamide, poly(hydroxyethyl acrylate), poly(hydroxyethyl methacrylate), poly(glycolic acid), poly(DL-lactic-co-glycolic acid), polyhydroxyalkanoate, poly(4-hydroxybutirate), sulfonated polymers, polygluconic acid, poly(acrylic acid), polyphosphazenes, polysaccharides, proteins, collagen, elastin, alginate, fibrin, fibronectin, laminin, hyaluronic acid, and polypeptide sequences cleavable by proteases.

57. The method of claim 50, wherein the hydrogel comprises polyethylene glycol sulfhydryl (PEG-SH) and polyethylene glycol vinyl sulfone (PEG-VS).

58. The method of claim 50, wherein said varying the elasticity of the substrate of step (b) occurs after adding an agent, cell, chemical, drug, or biomolecule.

59. The method of claim 50, wherein said cell is a stem cell, primary cell, transdifferentiated cell, dedifferentiated cell, reprogrammed cell, multipotent cell, gamete, or pluripotent cell.

60. The method of claim 50, wherein said cell is a somatic cell.

61. The method of claim 50, wherein said cell is a muscle cell, hematopoietic cell, neural cell, mesenchymal cell, pancreatic cell, hepatic cell, cardiac cell, kidney cell, liver cell, skeletal muscle cell, mammary fatty tissue cell, mammary gland cell, endothelial cell, adipose tissue cell (e.g., adipocyte), thyroid cell, articular cartilage, skin cell, prostate cell, lymph node cell, blood cell, retinal cell, dental pulp cell, bladder cell, spleen cell, small intestine cell, colon cell, rectal cell, lung cell, hair follicle cell, intestinal cell, or bone marrow cell.

62. The method of any of claim 1 or 50, wherein said cell is a pancreatic cell.

63. The method of claim 62, wherein the pancreatic cell is a pancreatic islet cell.

64. The method of claim 63, wherein the pancreatic islet cell is a pancreatic beta cell.

65. The method of claim 63, wherein the pancreatic islet cell is a pancreatic alpha cell.

66. A composition comprising an elastic substrate and a pancreatic cell, wherein the elastic substrate is the hydrogel of any one of claim 34 or 41.

67. The composition of claim 66, wherein the pancreatic cell is a pancreatic islet cell.

68. The composition of claim 67, wherein the pancreatic islet cell is a pancreatic islet beta cell.
